SEQUENCE LISTING (46 sequences)

SEQ ID NO: 1 - protein, 37 aa, synthetic construct
DWLKAFYDKVAEKLKEAFPDWAKAAYDKAAEKAKEAA

SEQ ID NO: 2 - protein, 37 aa, synthetic construct
DWLKAFYDKVAEKLKEAFPDWLKAFYDKVAEKLKEAF

SEQ ID NO: 3 - protein, 42 aa, synthetic construct
RMRITERDDFRGQMSEITDDCPSLQDRFHLTEVHSLRVLEGS

SEQ ID NO: 4 - protein, 37 aa, synthetic construct
DWLKAFYDKVAEKLKEAFPDWLKAFYDKVAEKAKEAF

SEQ ID NO: 5 - protein, 37 aa, synthetic construct
DWLKAFYDKVAEKLKEAFPDWLKAFYDKVAEKAKEAA

SEQ ID NO: 6 - protein, 37 aa, synthetic construct
DWLKAFYDKVAEKLKEAFPDWLKAAYDKVAEKAKEAA

SEQ ID NO: 7 - protein, 37 aa, synthetic construct
DWLKAFYDKVAEKLKEAFPDWLKAAYDKAAEKAKEAA

SEQ ID NO: 8 - protein, 37 aa, synthetic construct
DWLKAFYDKVAEKLKEAFPDWGKAGYDKGAEKGKEAG

SEQ ID NO: 9 - protein, 37 aa, synthetic construct
DWLKAFYDKVAEKLKEAFPDWGKAGYDKGAEKGKEAF

SEQ ID NO: 10 - protein, 36 aa, synthetic construct
DWGKAGYDKGAEKGKEAGDWLKAFYDKVAEKLKEAF

SEQ ID NO: 11 - protein, 34 aa, synthetic construct
DWLKAFYDKVAEKLKEAFPDWLKAFYDKVAEKLK

SEQ ID NO: 12 - protein, 34 aa, synthetic construct
KAFYDKVAEKLKEAFPDWLKAFYDKVAEKLKEAF

SEQ ID NO: 13 - protein, 30 aa, synthetic construct
DWLKAFYDKVAEKLKEAFPDWLKAFYDKVA

SEQ ID NO: 14 - protein, 30 aa, synthetic construct
DKVAEKLKEAFPDWLKAFYDKVAEKLKEAF

SEQ ID NO: 15 - protein, 36 aa, synthetic construct
DWLKAFYDKVAEKLKEAFPDWLKAFYKVAEKLKEAF

SEQ ID NO: 16 - protein, 35 aa, synthetic construct
DWLKAFYDKVAEKLKEAFPDWLKAFYVAEKLKEAF

SEQ ID NO: 17 - protein, 36 aa, synthetic construct
DWLAFYDKVAEKLKEAFPDWLKAFYDKVAEKLKEAF

SEQ ID NO: 18 - protein, 35 aa, synthetic construct
DWLFYDKVAEKLKEAFPDWLKAFYDKVAEKLKEAF

SEQ ID NO: 19 - protein, 38 aa, synthetic construct
DWLKAFYDKVAEKLKEAFPDWLAKAFYDKVAEKLKEAF

SEQ ID NO: 20 - protein, 39 aa, synthetic construct
DWLKAFYDKVAEKLKEAFPDWLAAKAFYDKVAEKLKEAF

SEQ ID NO: 21 - protein, 38 aa, synthetic construct
DWLKAAFYDKVAEKLKEAFPDWLKAFYDKVAEKLKEAF

SEQ ID NO: 22 - protein, 39 aa, synthetic construct
DWLKAAAFYDKVAEKLKEAFPDWLKAFYDKVAEKLKEAF

SEQ ID NO: 23 - protein, 37 aa, synthetic construct
DWLKAFYDKVAEKLKEAFPDWLEAFYDKVAKKLKEAF

SEQ ID NO: 24 - protein, 37 aa, synthetic construct
DWLKAFYDKVAEKLKEAFPDWLEAFYDEVAKKLKKAF

SEQ ID NO: 25 - protein, 37 aa, synthetic construct
DWLEAFYDKVAKKLKEAFPDWLKAFYDKVAEKLKEAF

SEQ ID NO: 26 - protein, 37 aa, synthetic construct
DWLEAFYDEVAKKLKKAFPDWLKAFYDKVAEKLKEAF

SEQ ID NO: 27 - protein, 37 aa, synthetic construct; identical to SEQ ID NO: 2

SEQ ID NO: 28 - protein, 37 aa, synthetic construct; identical to SEQ ID NO: 2

SEQ ID NO: 29 - protein, 37 aa, synthetic construct; identical to SEQ ID NO: 2

SEQ ID NO: 30 - protein, 37 aa, synthetic construct; identical to SEQ ID NO: 2

SEQ ID NO: 31 - protein, 37 aa, synthetic construct
LLDNWDSVTSTFSKLREQPDWAKAAYDKAAEKAKEAA

SEQ ID NO: 32 - protein, 37 aa, synthetic construct
LESFKVSFLSALEEYTKKPDWAKAAYDKAAEKAKEAA

SEQ ID NO: 33 - protein, 38 aa, synthetic construct
DWAKAAYDKAAEICAKEAAPLLDNWDSVTSTFSKLREQ

SEQ ID NO: 34 - protein, 37 aa, synthetic construct
DWAKAAYDKAAEKAKEAAPLESFKVSFLSALEEYTKK

SEQ ID NO: 35 - protein, 37 aa, synthetic construct
DWLKAFYDKVAEKLKEAFPSDELRQRLAARLEALKEN

SEQ ID NO: 36 - protein, 37 aa, synthetic construct
DWLKAFYDKVAEKLKEAFPRAELQEGARQKLHELQEK

SEQ ID NO: 37 - protein, 37 aa, synthetic construct
SDELRQRLAARLEALKENPDWLKAFYDKVAEKLKEAF

SEQ ID NO: 38 - protein, 37 aa, synthetic construct
RAELQEGARQKLHELQEKPDWLKAFYDKVAEKLKEAF

SEQ ID NO: 39 - protein, 37 aa, synthetic construct [sequence omitted]

SEQ ID NO: 40 - protein, 37 aa, synthetic construct [sequence omitted]

SEQ ID NO: 41 - protein, 37 aa, synthetic construct
SDELRQRLAARLEALKENPLLDNWDSVTSTFSKLREQ

SEQ ID NO: 42 - protein, 37 aa, synthetic construct
LLDNWDSVTSTFSKLREQPLESFKVSFLSALEEYTKK

SEQ ID NO: 43 - protein, 37 aa, synthetic construct
DWLKAFYDKVAEKLKEAFPDWLRAFYDKVAEKLKEAF

SEQ ID NO: 44 - protein, 37 aa, synthetic construct
DWLKAFYDKVAEKLKEAFPDWLRAFYDRVAEKLKEAF

SEQ ID NO: 45 - protein, 37 aa, synthetic construct
DWLKAFYDKVAEKLKEAFPDWLRAFYDRVAEKLREAF

SEQ ID NO: 46 - protein, 5 aa, synthetic construct
  SITE 1: note = palmitoyl-Lysine
KTTKS

(19) United States
(12) Patent Application Publication
(10) Pub. No.: US 2024/0366521 A1
(43) Pub. Date: Nov. 7, 2024

(54) NANOPARTICLES AND METHODS OF USE

(71) Applicant: Qana Therapeutics Inc., Austin, TX (US)

(72) Inventors: James Graham, Austin, TX (US); Christian Boada, Austin, TX (US)

(21) Appl. No.: 18/415,494

(22) Filed: Jan. 17, 2024

Related U.S. Application Data
(60) Provisional application No. 63/480,269, filed on Jan. 17, 2023.

(51) Int. Cl. A61K 9/51; A61K 31/40; A61K 31/496; A61K 31/498; A61K 31/704

(52) U.S. Cl. CPC A61K 9/5169; A61K 9/5123; A61K 31/40; A61K 31/496; A61K 31/498; A61K 31/704

(57) ABSTRACT

The present disclosure relates to nanoparticles comprising a payload and a conjugate having the structure of formula (I):

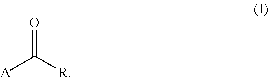

### CROSS-REFERENCE TO RELATED APPLICATIONS

This application claims the benefit of and priority to U.S. Provisional Application No. 63/480,269, filed Jan. 17, 2023, the contents of which are incorporated herein by reference in its entirety.

### SEQUENCE LISTING

The instant application contains a Sequence Listing which is hereby incorporated by reference in its entirety. Said XML copy, created Apr. 10, 2024, is named QTP-001US_SL.xml, and is 42,408 bytes in size.

### SUMMARY OF THE DISCLOSURE

Disclosed herein, in certain embodiments, are nanoparticles, comprising: (a) a payload; and (b) a conjugate having the structure of formula (I):

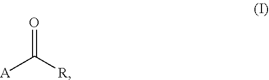

wherein
A is a peptide; and
R is C₁₋₄₀ alkyl, C₂₋₄₀ alkenyl, or C₂₋₄₀ alkynyl.

In some embodiments, the nanoparticles have a particle size of about 5 nm to about 175 nm. In some embodiments, the nanoparticles have a particle size of about 5 nm to about 55 nm. In some embodiments, the nanoparticles have a particle size of about 55 nm to about 115 nm. In some embodiments, the nanoparticles have a particle size of about 115 nm to about 175 nm. In some embodiments, the nanoparticles have a particle size of about 175 nm to about 350 nm. In some embodiments, the nanoparticles have a payload:conjugate molar ratio of about 1:20 to about 1:1. In some embodiments, the payload:conjugate molar ratio is about 1:10. In some embodiments, the payload:conjugate molar ratio is about 1:7. In some embodiments, the payload:conjugate molar ratio is about 1:5. In some embodiments, the payload:conjugate molar ratio is about 1:3. In some embodiments, R is C₆₋₄₀ alkyl, C₆₋₄₀ alkenyl, or C₆₋₄₀ alkynyl. In some embodiments, R is C₆₋₂₀ alkyl, C₆₋₂₀ alkenyl, or C₆₋₂₀ alkynyl. In some embodiments, R is C₆₋₂₀ alkyl. In some embodiments, R is:

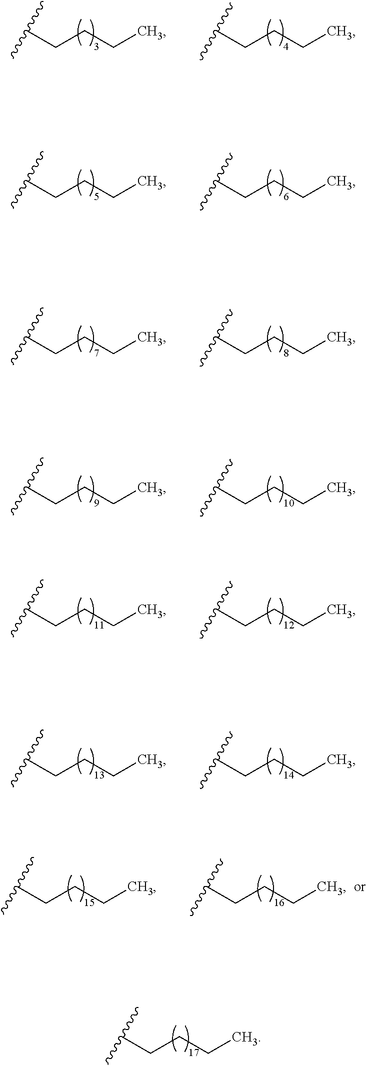

In some embodiments, R is

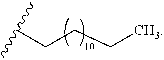

In some embodiments, the peptide is an apolipoprotein. In some embodiments, the apolipoprotein is apolipoprotein A-I, apolipoprotein A-II, apolipoprotein A-IV, apolipoprotein A-V, apolipoprotein B, apolipoprotein C, apolipoprotein D, or apolipoprotein E. In some embodiments, the apolipoprotein is apolipoprotein A-I. In some embodiments, the peptide is an apolipoprotein mimetic. In some embodiments, the apolipoprotein mimetic is apolipoprotein A-I mimetic, apolipoprotein A-II mimetic, apolipoprotein A-IV mimetic, apolipoprotein A-V mimetic, apolipoprotein B mimetic, apolipoprotein C mimetic, apolipoprotein D mimetic, or apolipoprotein E mimetic. In some embodiments, the apolipoprotein mimetic is apolipoprotein A-I mimetic. In some embodiments, the apolipoprotein mimetic comprises an amino acid sequence having at least about 70% sequence identity to the amino acid sequence of any one of SEQ ID NOs: 1-45. In some embodiments, the apolipoprotein mimetic comprises an amino acid sequence having at least 75%, at least 80%, at least 85%, at least 90%, at least 95%, at least 96%, at least 97%, at least 98%, or at least 99% sequence identity to any one of SEQ ID NOs: 1-45. In some embodiments, the apolipoprotein mimetic comprises an amino acid sequence having at least about 70% sequence identity to the amino acid sequence of SEQ ID NO: 1. In some embodiments, the apolipoprotein mimetic comprises an amino acid sequence having at least 75%, at least 80%, at least 85%, at least 90%, at least 95%, at least 96%, at least 97%, at least 98%, or at least 99% sequence identity to SEQ ID NO: 1. In some embodiments, the payload is a hydrophobic molecule. In some embodiments, the payload is a therapeutic agent. In some embodiments, the therapeutic agent is a chemotherapeutic agent. In some embodiments, the therapeutic agent is an anthracycline, an anthracenedione, a bleomycin, or a mitomycin. In some embodiments, the therapeutic agent is an alkaloid, a monomethyl auristatin, or a taxane. In some embodiments, the therapeutic agent is a quinazolinone. In some embodiments, the therapeutic agent is a cyclic dinucleotide, a macrocycle-bridged STING agonist, or a xanthone. In some embodiments, the therapeutic agent is a adenosine derivative, a guanosine derivative, a cytidine derivative, a uridine derivative, or a thymidine derivative. In some embodiments, the therapeutic agent is cyclophosphamide, chlorambucil, cisplatin, etoposide, ametantrone, bleomycin, dactinomycin, daunorubicin, doxorubicin, epirubicin, idarubicin, mitomycin C, mitoxantrone, N-benzyladriamycin-14-valerate (AD198), valrubicin, docetaxel, monomethyl auristatin E (MMAE), fluorenylmethoxycarbonyl-monomethyl auristatin E (Fmoc-MMAE), monomethyl auristatin F (MMAF), paclitaxel, vinblastine, vincristine, vindesine, vinorelbine, bardoxolone methyl, curcumin, deferasirox, deferoxamine mesylate, erastin, imidazole ketone erastin (IKE), lapatinib,linagliptin, nordihydroguaiaretic acid (NDGA), pioglitazone, rosadustat, rosiglitazone, setanaxib, simvastatin, sorafenib, sulfasalazine, troglitazone, zileuton, RSL3, ML162, ML210, everolimus, rapamycin, ridaforolimus, sirolimus, temsirolimus, altretamine, bendamustine, busulfan, carboplatin, carmustine, dacarbazine, ifosfamide, lurbinectedin, lomustine, mechlorethamine, melphalan, oxaliplatin, temozolomide, thiotepa, trabectedin, camptothecin, 7-ethyl-10-hydroxycamptothecin (SN-38), exatecan, gimatecan, irinotecan, karenitecin, lurtotecan, rubitecan, silatecan, topotecan, diflomotecan, (S)-13-cyclobutyl-7-ethyl-7-hydroxy-9,12-dihydro-7H-cyclopenta[6,7]indolizino[1,2-b][1,3]dioxolo[4,5-g]quinoline-8,10-dione (S3925), cabazitaxel, eribulin, ixabepilone, tirbanibulin, niraparib, olaparib, pamiparib, rucaparib, talazoparib, veliparib, dimethylxanthone acetic acid (DMXAA or vadimezan), ADU-S100, E7766, MK-1454, acelarin, capecitabine, gemcitabine, sapacitabine, ML210, or a pharmaceutically acceptable salt thereof. In some embodiments, the therapeutic agent is N-benzyladriamycin-14-valerate (AD198), monomethyl auristatin E (MMAE), fluorenylmethoxycarbonyl-monomethyl auristatin E (Fmoc-MMAE), erastin, imidazole ketone erastin (IKE), rapamycin, lurbinectedin, trabectedin, irinotecan, 7-ethyl-10-hydroxycamptothecin (SN-38), paclitaxel, vincristine, rucaparib, dimethylxanthone acetic acid (DMXAA or vadimezan), acelarin, ML210, or a pharmaceutically acceptable salt thereof. In some embodiments, the payload is an diagnostic agent. In some embodiments, the diagnostic agent is an imaging agent. In some embodiments, the imaging agent comprises a fluorescent dye. In some embodiments, the fluorescent dye is a xanthene, bimane, coumarin, aromatic amine, benzofuran, fluorescent cyanine, indocarbocyanine, carbazole, dicyanomethylene pyrane, polymethine, oxabenzanthrane, xanthene, pyrylium, carbostyl, perylene, acridone, quinacridone, rubrene, anthracene, coronene, phenanthrecene, pyrene, butadiene, stilbene, porphyrin, pthalocyanine, lanthanide metal chelate complexes, rare-earth metal chelate complexes, or derivatives thereof. In some embodiments, the fluorescent dye is 2-[2-[2-Chloro-3-[(1,3-dihydro-3,3-dimethyl-1-propyl-2H-indol-2-ylidene)ethylidene]-1-cyclohexen-1-yl]ethenyl]-3,3-dimethyl-1-propylindolium iodide (IR780) or a pharmaceutically acceptable salt thereof. In some embodiments, the nanoparticle further comprises an excipient. In some embodiments, the excipient is a lipid. In some embodiments, the lipid is a phospholipid. In some embodiments, the phospholipid is phosphatidylcholine (PC), lysophosphatidylcholine (LPC), phosphatidic acid (PA), lysophosphatidic acid (LPA), phosphatidylethanolamine (PE), lysophosphatidylethanolamine (LPE), phosphatidylglycerol (PG), lysophosphatidylglycerol (LPG), phosphoinositides (PI), lysophosphatidylinositol (LPI), phosphatidylserine (PS), or lysophosphatidylserine (LPS). In some embodiments, the phospholipid is 1,2-dihexanoyl-sn-glycero-3-phosphocholine (DHPC) or 1,2-dimyristoyl-sn-glycero-3-phosphocholine (DMPC). In some embodiments, the lipid is a lipopeptide. In some embodiments, the lipopeptide is a micelle-forming lipopeptide. In some embodiments, the lipopeptide is a palmitoylated peptide. In some embodiments, the lipopeptide is palmitoyl pentapeptide-4 (Pal-KTTKS(SEQ ID NO: 46)). In some embodiments, the nanoparticles have a payload:lipid molar ratio of about 1:20 to about 1:1. In some embodiments, the payload:lipid molar ratio is about 1:10. In some embodiments, the payload:lipid molar ratio is about 1:7. In some embodiments, the payload:lipid molar ratio is about 1:5. In some embodiments, the payload:lipid molar ratio is about 1:3.

Disclosed herein, in certain embodiments, are pluralities of nanoparticles, comprising: (a) a payload; and (b) a conjugate comprising the structure of formula (I):

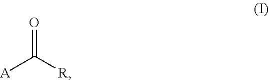

wherein
A is a peptide; and
R is C₁₋₄₀ alkyl, C₂₋₄₀ alkenyl, or C₂₋₄₀ alkynyl.

In some embodiments, the plurality of nanoparticles comprises less than about 5% impurities. In some embodiments, the plurality of nanoparticles comprises less than about 4% impurities, less than about 3% impurities, less than about 2% impurities, less than about 1% impurities, or less than about 0.5% impurities. In some embodiments, the plurality of nanoparticles has a mean particle size of about 5 nm to about 175 nm. In some embodiments, the plurality of nanoparticles has a mean particle size of about 5 nm to about 15 nm; about 15 nm to about 25 nm; about 25 nm to about 35 nm; about 35 nm to about 45 nm; about 45 nm to about 55 nm; about 55 nm to about 105 nm; about 55 nm to about 65 nm; about 55 nm to about 85 nm; about 65 nm to about 75 nm; about 75 nm to about 85 nm; about 85 nm to about 95 nm; about 85 nm to about 115 nm; about 95 nm to about 105 nm; about 105 nm to about 175 nm; about 105 nm to about 155 nm; about 105 nm to about 115 nm; about 115 nm to about 125 nm; about 115 nm to about 145 nm; about 125 nm to about 135 nm; about 135 nm to about 145 nm; about 145 nm to about 155 nm; about 145 nm to about 175 nm; about 155 nm to about 165 nm; or about 165 nm to about 175 nm. In some embodiments, the plurality of nanoparticles has a polydispersity index less than 0.5. In some embodiments, the polydispersity index is less than 0.45, less than 0.4, less than 0.35, less than 0.3, less than 0.25, less than 0.2, less than 0.15, less than 0.1, or less than 0.05. In some embodiments, 90% of the nanoparticles have a particle size less than 175 nm. In some embodiments, 90% of the nanoparticles have a particle size less than 170 nm, less than 165 nm, less than 160 nm, less than 155 nm, less than 150 nm, less than 145 nm, less than 140 nm, less than 135 nm, less than 130 nm, less than 125 nm, less than 120 nm, less than 115 nm, less than 110 nm, less than 105 nm, less than 100 nm, less than 95 nm, less than 90 nm, less than 85 nm, less than 80 nm, less than 75 nm, less than 70 nm, less than 65 nm, less than 60 nm, less than 55 nm, less than 50 nm, less than 45 nm, less than 40 nm, less than 35 nm, less than 30 nm, less than 25 nm, less than 20 nm, less than 15 nm, or less than 5 nm. In some embodiments, R is C₆₋₄₀ alkyl, C₆₋₄₀ alkenyl, or C₆₋₄₀ alkynyl. In some embodiments, R is C₆₋₂₀ alkyl, C₆₋₂₀ alkenyl, or C₆₋₂₀ alkynyl. In some embodiments, R is C₆₋₂₀ alkyl. In some embodiments, R is:

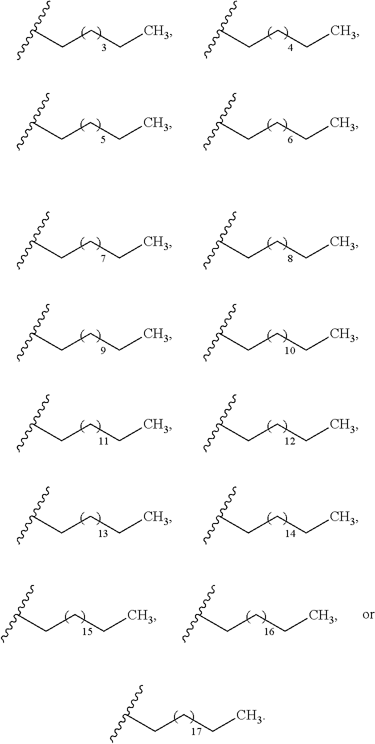

In some embodiments, R is

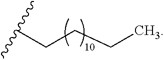

In some embodiments, the peptide is an apolipoprotein. In some embodiments, the apolipoprotein is apolipoprotein A-I, apolipoprotein A-II, apolipoprotein A-IV, apolipoprotein A-V, apolipoprotein B, apolipoprotein C, apolipoprotein D, or apolipoprotein E. In some embodiments, the apolipoprotein is apolipoprotein A-I. In some embodiments, the peptide is an apolipoprotein mimetic. In some embodiments, the apolipoprotein mimetic is apolipoprotein A-I mimetic, apolipoprotein A-II mimetic, apolipoprotein A-IV mimetic, apolipoprotein A-V mimetic, apolipoprotein B mimetic, apolipoprotein C mimetic, apolipoprotein D mimetic, or apolipoprotein E mimetic. In some embodiments, the apolipoprotein mimetic is apolipoprotein A-I mimetic. In some embodiments, the apolipoprotein mimetic comprises an amino acid sequence having at least about 70% sequence identity to the amino acid sequence of any one of SEQ ID NOs: 1-45. In some embodiments, the apolipoprotein mimetic comprises an amino acid sequence having at least 75%, at least 80%, at least 85%, at least 90%, at least 95%, at least 96%, at least 97%, at least 98%, or at least 99% sequence identity to any one of SEQ ID NOs: 1-45. In some embodiments, the apolipoprotein mimetic comprises an amino acid sequence having at least about 70% sequence identity to the amino acid sequence of SEQ ID NO: 1. In some embodiments, the apolipoprotein mimetic comprises an amino acid sequence having at least 75%, at least 80%, at least 85%, at least 90%, at least 95%, at least 96%, at least 97%, at least 98%, or at least 99% sequence identity to SEQ ID NO: 1. In some embodiments, the plurality of nanoparticles has a mean particle size of about 165 nm to about 175 nm and a polydispersity index less than 0.2, and wherein 90% of the nanoparticles of the plurality of nanoparticles have a particle size less than 115 nm. In some embodiments, the plurality of nanoparticles has a mean particle size of about 145 nm to about 155 nm and a polydispersity index less than 0.3, and wherein 90% of the nanoparticles of the plurality of nanoparticles have a particle size less than 65 nm. In some embodiments, the plurality of nanoparticles has a mean particle size of about 135 nm to about 145 nm and a polydispersity index less than 0.25, and wherein 90% of the nanoparticles of the plurality of nanoparticles have a particle size less than 85 nm. In some embodiments, the plurality of nanoparticles has a mean particle size of about 125 nm to about 135 nm and a polydispersity index less than 0.3, and wherein 90% of the nanoparticles of the plurality of nanoparticles have a particle size less than 135 nm. In some embodiments, the plurality of nanoparticles has a mean particle size of about 115 nm to about 125 nm and a polydispersity index less than 0.25, and wherein 90% of the plurality of nanoparticles have a particle size less than 105 nm. In some embodiments, the plurality of nanoparticles has a mean particle size of about plurality of nanoparticles is about 105 nm to about 115 nm and a polydispersity index less than 0.3, and wherein 90% of the nanoparticles of the plurality of nanoparticles have a particle size less than 75 nm. In some embodiments, the plurality of nanoparticles has a mean particle size of about plurality of nanoparticles is about 95 nm to about 105 nm and a polydispersity index less than 0.25, and wherein 90% of the nanoparticles of the plurality of nanoparticles have a particle size less than 55 nm. In some embodiments, the plurality of nanoparticles has a mean particle size of about plurality of nanoparticles is about 85 nm to about 95 nm and a polydispersity index less than 0.25, and wherein 90% of the nanoparticles of the plurality of nanoparticles have a particle size less than 55 nm. In some embodiments, the plurality of nanoparticles has a mean particle size of about plurality of nanoparticles is about 85 nm to about 95 nm and a polydispersity index less than 0.3, and wherein 90% of the nanoparticles of the plurality of nanoparticles have a particle size less than 75 nm. In some embodiments, the plurality of nanoparticles has a mean particle size of about plurality of nanoparticles is about 85 nm to about 95 nm and a polydispersity index less than 0.3, and wherein 90% of the nanoparticles of the plurality of nanoparticles have a particle size less than 125 nm. In some embodiments, the plurality of nanoparticles has a mean particle size of about plurality of nanoparticles is about 75 nm to about 85 nm and a polydispersity index less than 0.3, and wherein 90% of the nanoparticles of the plurality of nanoparticles have a particle size less than 125 nm. In some embodiments, the plurality of nanoparticles has a mean particle size of about plurality of nanoparticles is about 55 nm to about 65 nm and a polydispersity index less than 0.1, and wherein 90% of the nanoparticles of the plurality of nanoparticles have a particle size less than 75 nm. In some embodiments, the payload is a hydrophobic molecule. In some embodiments, the payload is a therapeutic agent. In some embodiments, the therapeutic agent is a chemotherapeutic agent. In some embodiments, the chemotherapeutic agent is a DNA intercalator, a mitotic inhibitor, a ferroptosis inducer, a kinase inhibitor, an alkylating agent, a topoisomerase inhibitor, a microtubule inhibitor, a poly adenosine diphosphate ribose polymerase (poly(ADP-ribose) polymerase or PARP) inhibitor, a stimulator of interferon genes (STING) agonist, or a nucleoside analog. In some embodiments, the therapeutic agent is a ferroptosis inhibitor. In some embodiments, the therapeutic agent is an anthracycline, an anthracenedione, a bleomycin, or a mitomycin. In some embodiments, the therapeutic agent is an alkaloid, a monomethyl auristatin, or a taxane. In some embodiments, the therapeutic agent is a quinazolinone. In some embodiments, the therapeutic agent is a cyclic dinucleotide, a macrocycle-bridged STING agonist, or a xanthone. In some embodiments, the therapeutic agent is a adenosine derivative, a guanosine derivative, a cytidine derivative, a uridine derivative, or a thymidine derivative. In some embodiments, the therapeutic agent is cyclophosphamide, chlorambucil, cisplatin, etoposide, ametantrone, bleomycin, dactinomycin, daunorubicin, doxorubicin, epirubicin, idarubicin, mitomycin C, mitoxantrone, N-benzyladriamycin-14-valerate (AD198), valrubicin, docetaxel, monomethyl auristatin E (MMAE), fluorenylmethoxycarbonyl-monomethyl auristatin E (Fmoc-MMAE), monomethyl auristatin F (MMAF), paclitaxel, vinblastine, vincristine, vindesine, vinorelbine, bardoxolone methyl, curcumin, deferasirox, deferoxamine mesylate, erastin, imidazole ketone erastin (IKE), lapatinib,linagliptin, nordihydroguaiaretic acid (NDGA), pioglitazone, rosadustat, rosiglitazone, setanaxib, simvastatin, sorafenib, sulfasalazine, troglitazone, zileuton, RSL3, ML162, ML210, everolimus, rapamycin, ridaforolimus, sirolimus, temsirolimus, altretamine, bendamustine, busulfan, carboplatin, carmustine, dacarbazine, ifosfamide, lurbinectedin, lomustine, mechlorethamine, melphalan, oxaliplatin, temozolomide, thiotepa, trabectedin, camptothecin, 7-ethyl-10-hydroxycamptothecin (SN-38), exatecan, gimatecan, irinotecan, karenitecin, lurtotecan, rubitecan, silatecan, topotecan, diflomotecan, (S)-13-cyclobutyl-7-ethyl-7-hydroxy-9,12-dihydro-7H-cyclopenta[6,7]indolizino[1,2-b][1,3]dioxolo[4,5-g]quinoline-8,10-dione (S3925), cabazitaxel, eribulin, ixabepilone, tirbanibulin, niraparib, olaparib, pamiparib, rucaparib, talazoparib, veliparib, dimethylxanthone acetic acid (DMXAA or vadimezan), ADU-S100, E7766, MK-1454, acelarin, capecitabine, gemcitabine, sapacitabine, ML210, or a pharmaceutically acceptable salt thereof. In some embodiments, the therapeutic agent is N-benzyladriamycin-14-valerate (AD198), monomethyl auristatin E (MMAE), fluorenylmethoxycarbonyl-monomethyl auristatin E (Fmoc-MMAE), erastin, imidazole ketone erastin (IKE), rapamycin, lurbinectedin, trabectedin, irinotecan, 7-ethyl-10-hydroxycamptothecin (SN-38), paclitaxel, vincristine, rucaparib, dimethylxanthone acetic acid (DMXAA or vadimezan), acelarin, ML210, or a pharmaceutically acceptable salt thereof. In some embodiments, the payload is an diagnostic agent. In some embodiments, the diagnostic agent is an imaging agent. In some embodiments, the imaging agent comprises a fluorescent dye. In some embodiments, the fluorescent dye is a xanthene, bimane, coumarin, aromatic amine, benzofuran, fluorescent cyanine, indocarbocyanine, carbazole, dicyanomethylene pyrane, polymethine, oxabenzanthrane, xanthene, pyrylium, carbostyl, perylene, acridone, quinacridone, rubrene, anthracene, coronene, phenanthrecene, pyrene, butadiene, stilbene, porphyrin, pthalocyanine, lanthanide metal chelate complexes, rare-earth metal chelate complexes, or derivatives thereof. In some embodiments, the fluorescent dye is 2-[2-[2-Chloro-3-[(1,3-dihydro-3,3-dimethyl-1-propyl-2H-indol-2-ylidene)ethylidene]-1-cyclohexen-1-yl]ethenyl]-3,3-dimethyl-1-propylindolium iodide (IR780) or a pharmaceutically acceptable salt thereof. In some embodiments, the nanoparticle further comprises an excipient. In some embodiments, the excipient is a lipid. In some embodiments, the lipid is a phospholipid. In some embodiments, the phospholipid is phosphatidylcholine (PC), lysophosphatidylcholine (LPC), phosphatidic acid (PA), lysophosphatidic acid (LPA), phosphatidylethanolamine (PE), lysophosphatidylethanolamine (LPE), phosphatidylglycerol (PG), lysophosphatidylglycerol (LPG), phosphoinositides (PI), lysophosphatidylinositol (LPI), phosphatidylserine (PS), or lysophosphatidylserine (LPS). In some embodiments, the phospholipid is 1,2-dihexanoyl-sn-glycero-3-phosphocholine (DHPC) or 1,2-dimyristoyl-sn-glycero-3-phosphocholine (DMPC). In some embodiments, the lipid is a lipopeptide. In some embodiments, the lipopeptide is a micelle-forming lipopeptide. In some embodiments, the lipopeptide is a palmitoylated peptide. In some embodiments, the lipopeptide is palmitoyl pentapeptide-4 (Pal-KTTKS(SEQ ID NO: 46)).

Disclosed herein, in certain embodiments, are pharmaceutical compositions, comprising: (a) a plurality of nanoparticles comprising: (i) a payload; and (ii) a conjugate comprising the structure of formula (I):

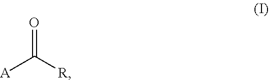

wherein
A is a peptide; and
R is C₁₋₄₀ alkyl, C₂₋₄₀ alkenyl, or C₂₋₄₀ alkynyl; and
   (b) a pharmaceutically acceptable excipient. In some embodiments, the pharmaceutical composition comprises less than about 5% impurities. In some embodiments, the pharmaceutical composition comprises less than about 4% impurities, less than about 3% impurities, less than about 2% impurities, less than about 1% impurities, or less than about 0.5% impurities. In some embodiments, the plurality of nanoparticles has a mean particle size of about 5 nm to about 175 nm. In some embodiments, the mean particle size is about 5 nm to about 15 nm; about 15 nm to about 25 nm; about 25 nm to about 35 nm; about 35 nm to about 45 nm; about 45 nm to about 55 nm; about 55 nm to about 105 nm; about 55 nm to about 65 nm; about 55 nm to about 85 nm; about 65 nm to about 75 nm; about 75 nm to about 85 nm; about 85 nm to about 95 nm; about 85 nm to about 115 nm; about 95 nm to about 105 nm; about 105 nm to about 175 nm; about 105 nm to about 155 nm; about 105 nm to about 115 nm; about 115 nm to about 125 nm; about 115 nm to about 145 nm; about 125 nm to about 135 nm; about 135 nm to about 145 nm; about 145 nm to about 155 nm; about 145 nm to about 175 nm; about 155 nm to about 165 nm; or about 165 nm to about 175 nm. In some embodiments, the plurality of nanoparticles has a polydispersity index less than 0.5. In some embodiments, the polydispersity index is less than 0.45, less than 0.4, less than 0.35, less than 0.3, less than 0.25, less than 0.2, less than 0.15, less than 0.1, or less than 0.05. In some embodiments, 90% of the nanoparticles of the plurality of nanoparticles have a particle size less than 175 nm. In some embodiments, 90% of the nanoparticles of the plurality of nanoparticles have a particle size less than 170 nm, less than 165 nm, less than 160 nm, less than 155 nm, less than 150 nm, less than 145 nm, less than 140 nm, less than 135 nm, less than 130 nm, less than 125 nm, less than 120 nm, less than 115 nm, less than 110 nm, less than 105 nm, less than 100 nm, less than 95 nm, less than 90 nm, less than 85 nm, less than 80 nm, less than 75 nm, less than 70 nm, less than 65 nm, less than 60 nm, less than 55 nm, less than 50 nm, less than 45 nm, less than 40 nm, less than 35 nm, less than 30 nm, less than 25 nm, less than 20 nm, less than 15 nm, or less than 5 nm. In some embodiments, R is C₆₋₄₀ alkyl, C₆₋₄₀ alkenyl, or C₆₋₄₀ alkynyl. In some embodiments, R is C₆₋₂₀ alkyl, C₆₋₂₀ alkenyl, or C₆₋₂₀ alkynyl. In some embodiments, R is C₆₋₂₀ alkyl. In some embodiments, R is:

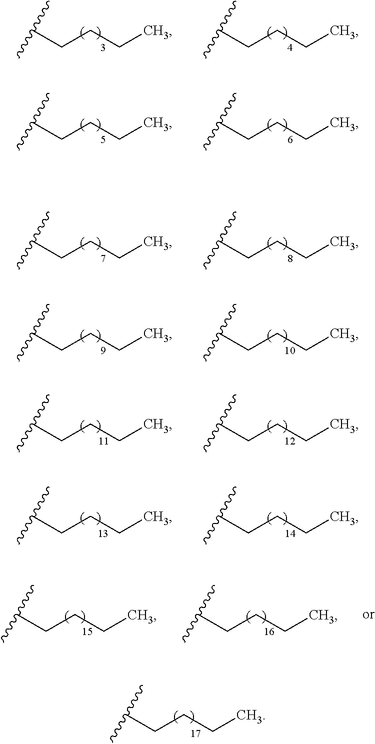

In some embodiments, R is

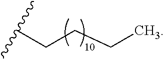

In some embodiments, the peptide is an apolipoprotein. In some embodiments, the apolipoprotein is apolipoprotein A-I, apolipoprotein A-II, apolipoprotein A-IV, apolipoprotein A-V, apolipoprotein B, apolipoprotein C, apolipoprotein D, or apolipoprotein E. In some embodiments, the apolipoprotein is apolipoprotein A-I. In some embodiments, the peptide is an apolipoprotein mimetic. In some embodiments, the apolipoprotein mimetic is apolipoprotein A-I mimetic, apolipoprotein A-II mimetic, apolipoprotein A-IV mimetic, apolipoprotein A-V mimetic, apolipoprotein B mimetic, apolipoprotein C mimetic, apolipoprotein D mimetic, or apolipoprotein E mimetic. In some embodiments, the apolipoprotein mimetic is apolipoprotein A-I mimetic. In some embodiments, the apolipoprotein mimetic comprises an amino acid sequence having at least about 70% sequence identity to the amino acid sequence of any one of SEQ ID NOs: 1-45. In some embodiments, the apolipoprotein mimetic comprises an amino acid sequence having at least 75%, at least 80%, at least 85%, at least 90%, at least 95%, at least 96%, at least 97%, at least 98%, or at least 99% sequence identity to any one of SEQ ID NOs: 1-45. In some embodiments, the apolipoprotein mimetic comprises an amino acid sequence having at least about 70% sequence identity to the amino acid sequence of SEQ ID NO: 1. In some embodiments, the apolipoprotein mimetic comprises an amino acid sequence having at least 75%, at least 80%, at least 85%, at least 90%, at least 95%, at least 96%, at least 97%, at least 98%, or at least 99% sequence identity to SEQ ID NO: 1. In some embodiments, the plurality of nanoparticles has a mean particle size of about 165 nm to about 175 nm and a polydispersity index less than 0.2, and wherein 90% of the nanoparticles of the plurality of nanoparticles have a particle size less than 115 nm. In some embodiments, the plurality of nanoparticles has a mean particle size of about 145 nm to about 155 nm and a polydispersity index less than 0.3, and wherein 90% of the nanoparticles of the plurality of nanoparticles have a particle size less than 65 nm. In some embodiments, the plurality of nanoparticles has a mean particle size of about 135 nm to about 145 nm and a polydispersity index less than 0.25, and wherein 90% of the nanoparticles of the plurality of nanoparticles have a particle size less than 85 nm. In some embodiments, the plurality of nanoparticles has a mean particle size of about 125 nm to about 135 nm and a polydispersity index less than 0.3, and wherein 90% of the nanoparticles of the plurality of nanoparticles have a particle size less than 135 nm. In some embodiments, the plurality of nanoparticles has a mean particle size of about 115 nm to about 125 nm and a polydispersity index less than 0.25, and wherein 90% of the plurality of nanoparticles have a particle size less than 105 nm. In some embodiments, the plurality of nanoparticles has a mean particle size of about plurality of nanoparticles is about 105 nm to about 115 nm and a polydispersity index less than 0.3, and wherein 90% of the nanoparticles of the plurality of nanoparticles have a particle size less than 75 nm. In some embodiments, the plurality of nanoparticles has a mean particle size of about plurality of nanoparticles is about 95 nm to about 105 nm and a polydispersity index less than 0.25, and wherein 90% of the nanoparticles of the plurality of nanoparticles have a particle size less than 55 nm. In some embodiments, the plurality of nanoparticles has a mean particle size of about plurality of nanoparticles is about 85 nm to about 95 nm and a polydispersity index less than 0.25, and wherein 90% of the nanoparticles of the plurality of nanoparticles have a particle size less than 55 nm. In some embodiments, the plurality of nanoparticles has a mean particle size of about plurality of nanoparticles is about 85 nm to about 95 nm and a polydispersity index less than 0.3, and wherein 90% of the nanoparticles of the plurality of nanoparticles have a particle size less than 75 nm. In some embodiments, the plurality of nanoparticles has a mean particle size of about plurality of nanoparticles is about 85 nm to about 95 nm and a polydispersity index less than 0.3, and wherein 90% of the nanoparticles of the plurality of nanoparticles have a particle size less than 125 nm. In some embodiments, the plurality of nanoparticles has a mean particle size of about plurality of nanoparticles is about 75 nm to about 85 nm and a polydispersity index less than 0.3, and wherein 90% of the nanoparticles of the plurality of nanoparticles have a particle size less than 125 nm. In some embodiments, the plurality of nanoparticles has a mean particle size of about plurality of nanoparticles is about 55 nm to about 65 nm and a polydispersity index less than 0.1, and wherein 90% of the nanoparticles of the plurality of nanoparticles have a particle size less than 75 nm. In some embodiments, the payload is a hydrophobic molecule. In some embodiments, the payload is a therapeutic agent. In some embodiments, the therapeutic agent is a chemotherapeutic agent. In some embodiments, the therapeutic agent is an anthracycline, an anthracenedione, a bleomycin, or a mitomycin. In some embodiments, the therapeutic agent is an alkaloid, a monomethyl auristatin, or a taxane. In some embodiments, the therapeutic agent is a quinazolinone. In some embodiments, the therapeutic agent is a cyclic dinucleotide, a macrocycle-bridged STING agonist, or a xanthone. In some embodiments, the therapeutic agent is a adenosine derivative, a guanosine derivative, a cytidine derivative, a uridine derivative, or a thymidine derivative. In some embodiments, the therapeutic agent is cyclophosphamide, chlorambucil, cisplatin, etoposide, ametantrone, bleomycin, dactinomycin, daunorubicin, doxorubicin, epirubicin, idarubicin, mitomycin C, mitoxantrone, N-benzyladriamycin-14-valerate (AD198), valrubicin, docetaxel, monomethyl auristatin E (MMAE), fluorenylmethoxycarbonyl-monomethyl auristatin E (Fmoc-MMAE), monomethyl auristatin F (MMAF), paclitaxel, vinblastine, vincristine, vindesine, vinorelbine, bardoxolone methyl, curcumin, deferasirox, deferoxamine mesylate, erastin, imidazole ketone erastin (IKE), lapatinib,linagliptin, nordihydroguaiaretic acid (NDGA), pioglitazone, rosadustat, rosiglitazone, setanaxib, simvastatin, sorafenib, sulfasalazine, troglitazone, zileuton, RSL3, ML162, ML210, everolimus, rapamycin, ridaforolimus, sirolimus, temsirolimus, altretamine, bendamustine, busulfan, carboplatin, carmustine, dacarbazine, ifosfamide, lurbinectedin, lomustine, mechlorethamine, melphalan, oxaliplatin, temozolomide, thiotepa, trabectedin, camptothecin, 7-ethyl-10-hydroxycamptothecin (SN-38), exatecan, gimatecan, irinotecan, karenitecin, lurtotecan, rubitecan, silatecan, topotecan, diflomotecan, (S)-13-cyclobutyl-7-ethyl-7-hydroxy-9,12-dihydro-7H-cyclopenta[6,7]indolizino[1,2-b][1,3]dioxolo[4,5-g]quinoline-8,10-dione (S3925), cabazitaxel, eribulin, ixabepilone, tirbanibulin, niraparib, olaparib, pamiparib, rucaparib, talazoparib, veliparib, dimethylxanthone acetic acid (DMXAA or vadimezan), ADU-S100, E7766, MK-1454, acelarin, capecitabine, gemcitabine, sapacitabine, ML210, or a pharmaceutically acceptable salt thereof. In some embodiments, the therapeutic agent is N-benzyladriamycin-14-valerate (AD198), monomethyl auristatin E (MMAE), fluorenylmethoxycarbonyl-monomethyl auristatin E (Fmoc-MMAE), erastin, imidazole ketone erastin (IKE), rapamycin, lurbinectedin, trabectedin, irinotecan, 7-ethyl-10-hydroxycamptothecin (SN-38), paclitaxel, vincristine, rucaparib, dimethylxanthone acetic acid (DMXAA or vadimezan), acelarin, ML210, or a pharmaceutically acceptable salt thereof. In some embodiments, the payload is an diagnostic agent. In some embodiments, the diagnostic agent is an imaging agent. In some embodiments, the imaging agent comprises a fluorescent dye. In some embodiments, the fluorescent dye is a xanthene, bimane, coumarin, aromatic amine, benzofuran, fluorescent cyanine, indocarbocyanine, carbazole, dicyanomethylene pyrane, polymethine, oxabenzanthrane, xanthene, pyrylium, carbostyl, perylene, acridone, quinacridone, rubrene, anthracene, coronene, phenanthrecene, pyrene, butadiene, stilbene, porphyrin, pthalocyanine, lanthanide metal chelate complexes, rare-earth metal chelate complexes, or derivatives thereof. In some embodiments, the fluorescent dye is 2-[2-[2-Chloro-3-[(1,3-dihydro-3,3-dimethyl-1-propyl-2H-indol-2-ylidene)ethylidene]-1-cyclohexen-1-yl]ethenyl]-3,3-dimethyl-1-propylindolium iodide (IR780) or a pharmaceutically acceptable salt thereof. In some embodiments, the nanoparticle further comprises a lipid. In some embodiments, the lipid is a phospholipid. In some embodiments, the phospholipid is phosphatidylcholine (PC), lysophosphatidylcholine (LPC), phosphatidic acid (PA), lysophosphatidic acid (LPA), phosphatidylethanolamine (PE), lysophosphatidylethanolamine (LPE), phosphatidylglycerol (PG), lysophosphatidylglycerol (LPG), phosphoinositides (PI), lysophosphatidylinositol (LPI), phosphatidylserine (PS), or lysophosphatidylserine (LPS). In some embodiments, the phospholipid is 1,2-dihexanoyl-sn-glycero-3-phosphocholine (DHPC) or 1,2-dimyristoyl-sn-glycero-3-phosphocholine (DMPC). In some embodiments, the lipid is a lipidated peptide. In some embodiments, the lipidated peptide is a palmitoylated peptide. In some embodiments, the lipidated peptide is palmitoyl pentapeptide-4 (Pal-KTTKS(SEQ ID NO: 46)).

Disclosed herein, in certain embodiments, are methods of synthesizing a plurality of nanoparticles disclosed herein, comprising combining a first composition comprising the conjugate and an aqueous solvent and a second composition comprising the payload and an organic solvent having a boiling point less than 65° C. at atmospheric pressure to form a mixture. In some embodiments, the combining comprises addition of the second composition to the first composition. In some embodiments, the combining comprises dropwise addition of the second composition to the first composition. In some embodiments, the organic solvent is a polar aprotic solvent. In some embodiments, the organic solvent is a halogenated solvent. In some embodiments, the organic solvent is dichloromethane, dichloroethane, chloroform, or a combination thereof. In some embodiments, the organic solvent is dichloromethane. In some embodiments, the organic solvent has a boiling point of about 40° C. to about 50° C., about 40° C. to about 45° C., about 45° C. to about 50° C., about 50° C. to about 60° C., about 50° C. to about 55° C., or about 55° C. to about 60° C. at atmospheric pressure. In some embodiments, the second composition further comprises a second organic solvent. In some embodiments, the second organic solvent is a polar aprotic solvent. In some embodiments, the second organic solvent is acetone, acetonitrile, ethyl acetate, ethanol, methanol, tetrahydrofuran, or a combination thereof. In some embodiments, the ratio of the organic solvent to the second organic solvent is about 50:50, about 55:45, about 60:40, about 65:35, about 70:30, about 75:25, about 80:20, about 85:15, about 90:10, or about 95:5. In some embodiments, the method further comprises heating the mixture to about 50° C. to about 60° C. In some embodiments, the method further comprises heating the mixture to about 55° C. to about 65° C. In some embodiments, the method further comprises heating the mixture to about 60° C. to about 70° C. In some embodiments, the method further comprises heating the mixture to about 65° C. to about 75° C. In some embodiments, the method further comprises heating the mixture to about 75° C. to about 80° C. In some embodiments, the method further comprises heating the mixture to about 80° C. to about 85° C. In some embodiments, the method further comprises heating the mixture to about 50° C. In some embodiments, the method further comprises heating the mixture to about 55° C. In some embodiments, the method further comprises heating the mixture to about 60° C. In some embodiments, the method further comprises heating the mixture to about 65° C. In some embodiments, the method further comprises heating the mixture to about 70° C. In some embodiments, the method further comprises heating the mixture to about 75° C. In some embodiments, the method further comprises heating the mixture to about 80° C. In some embodiments, the method further comprises heating the mixture to about 85° C. In some embodiments, the method further comprises lyophilizing the mixture. In some embodiments, the lyophilizing the mixture comprises adding trehalose to the mixture.

Disclosed herein, in certain embodiments, are methods of treating a disease or disorder in a subject in need thereof, comprising administering to the subject a therapeutically effective amount a pharmaceutical composition disclosed herein. In some embodiments, the subject is a pediatric subject. In some embodiments, the disease is cancer. In some embodiments, the cancer is selected from the group consisting of: bile duct cancer, bladder cancer, breast cancer, cervical cancer, colorectal cancer, esophageal cancer, gastrointestinal cancer, liver cancer, nasopharyngeal cancer, ovarian cancer, pancreatic cancer, prostate cancer, adrenocortical carcinoma, lung adenocarcinoma, renal cell carcinoma, leukemia, lymphoma, neuroblastoma, and sarcoma. In some embodiments, the renal cell carcinoma is clear cell renal carcinoma. In some embodiments, the sarcoma is a bone sarcoma. In some embodiments, the sarcoma is a soft tissue sarcoma. In some embodiments, the sarcoma is desmoplastic small round cell tumor (DSRCT), Ewing sarcoma, or rhabdomyosarcoma. In some embodiments, the cancer is a scavenger receptor class B type 1 (SR-B1) expressing cancer. In some embodiments, the cancer is associated with high expression of scavenger receptor class B type 1 (SR-B1). In some embodiments, the subject has elevated levels of scavenger receptor class B type 1 (SR-B1) as compared to a reference sample. In some embodiments, the method further comprises diagnosing the subject as having a cancer by determining the expression level of SR-B1.

Disclosed herein, in certain embodiments, are methods of detecting a disease in a subject in need thereof, comprising administering a therapeutically effective amount of a pharmaceutical composition disclosed herein. In some embodiments, the subject is a pediatric subject. In some embodiments, the disease is cancer. In some embodiments, the cancer is selected from the group consisting of bile duct cancer, bladder cancer, breast cancer, cervical cancer, colorectal cancer, esophageal cancer, gastrointestinal cancer, liver cancer, nasopharyngeal cancer, ovarian cancer, pancreatic cancer, prostate cancer, adrenocortical carcinoma, lung adenocarcinoma, renal cell carcinoma, leukemia, lymphoma, neuroblastoma, and sarcoma. In some embodiments, the renal cell carcinoma is clear cell renal carcinoma. In some embodiments, the sarcoma is a bone sarcoma. In some embodiments, the sarcoma is a soft tissue sarcoma. In some embodiments, the sarcoma is desmoplastic small round cell tumor (DSRCT), Ewing sarcoma, or rhabdomyosarcoma. In some embodiments, the cancer is a scavenger receptor class B type 1 (SR-B1) expressing cancer. In some embodiments, the cancer is associated with high expression of scavenger receptor class B type 1 (SR-B1). In some embodiments, the subject has elevated levels of scavenger receptor class B type 1 (SR-B1) as compared to a reference sample.

### Definitions

Unless defined otherwise, all technical and scientific terms used herein have the same meaning as is commonly understood by one of skill in the art to which the claimed subject matter belongs. Generally, nomenclatures utilized in connection with, and techniques of, nanoparticles and methods of synthesizing nanoparticles described herein are those well-known and commonly used in the art. It is to be understood that the foregoing general description and the following detailed description are exemplary and explanatory only and are not restrictive of any subject matter claimed. The section headings used herein are for organizational purposes only and are not to be construed as limiting the subject matter described.

Listed below are definitions of various terms used to describe the disclosure. These definitions apply to the terms as they are used throughout this specification and claims, unless otherwise limited in specific instances, either individually or as part of a larger group.

As used herein, the articles “a,” “an,” and “the” refer to one or more than one, unless indicated to the contrary or otherwise evident from the context. For example, reference to “a nanoparticle” includes a plurality of nanoparticles.

The terms “about” and “approximately” are to be construed as modifying a term or value such that it is not an absolute. This includes the degree of expected experimental error, technique error and instrument error for a given technique used to measure a value. When the term “about” is used before a quantitative value, the specific quantitative value itself is included, unless specifically stated otherwise. As used herein, the term “about” refers to a ±10% variation from the nominal value, unless otherwise indicated or inferred from the context.

The term “acyl,” as used herein, refers to a hydrocarbon radical having the following

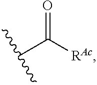

structure: wherein RA^{Ac} is hydrogen or carbon. Exemplary acyl groups include, but are not limited to, formyl, acetyl, propanoyl, butanoyl, pentanoyl, hexanoyl, heptanoyl, octanoyl, nonanoyl, decanoyl, undecanoyl, dodecanoyl, tridecanoyl, tetradecanoyl, pentadecenoyl, hexadecanoyl, heptadecanoyl, octadecanoyl, nonadecanoyl, eicosanoyl, and heneicosanoyl.

The term “alkyl,” as used herein, refers to saturated, straight- or branched-chain hydrocarbon radicals having 1-50 (e.g., 1-6, 1-8, 1-10, 1-12, 1-18, 1-20, 1-24, 1-30, 1-36, 2-20, 2-40, 6-20, or 6-40) carbon atoms. Examples of C₁₋₄₀ alkyl radicals include, but are not limited to, methyl, ethyl, propyl, isopropyl, n-butyl, tert-butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, and heneicosyl radicals.

The term “alkenyl,” as used herein, denotes a monovalent group derived from a hydrocarbon moiety containing from 2-50 carbon atoms having at least one carbon-carbon double bond. The double bond may or may not be the point of attachment to another group. Alkenyl groups include, but are not limited to, ethenyl, propenyl, butenyl, 1-methyl-2-buten-1-yl, heptenyl, and octenyl.

The term “alkynyl,” as used herein, denotes a monovalent group derived from a hydrocarbon moiety having 2-50 carbon atoms having at least one carbon-carbon triple bond. The triple bond may or may not be the point of attachment to another group. Alkynyl groups include, but are not limited to, ethynyl, propynyl, butynyl, 1-methyl-2-butyn-1-yl, heptynyl, and octynyl.

All ranges recited herein include the endpoints, including ranges that recite “between” two values. Additionally, all values or ranges include whole integers within or encompassing such ranges and fractions of the values or the integers within or encompassing ranges unless the context clearly indicates otherwise. For example, reference to a range of 90-100 nm, includes 91 nm, 92 nm, 93 nm, 94 nm, 95 nm, 95 nm, 97 nm, etc., as well as 91.1 nm, 91.2 nm, 91.3 nm, 91.4 nm, 91.5 nm, etc., 92.1 nm, 92.2 nm, 92.3 nm, 92.4 nm, 92.5 nm, etc., and so forth.

As used herein, the term “conjugate” refers to an entity formed by a peptide and a molecule that are covalently bonded (e.g., via an acyl group, e.g., a conjugate comprising the structure of formula (I) as described herein).

As used herein, the term “D90” refers to a value denoting the particle size threshold for 90% of the nanoparticles in a plurality of nanoparticles (i.e., 90% of the nanoparticles in a plurality of nanoparticles have a particle size below the D90 value). In some embodiments, the D90 value is used to characterize the particle size distribution of a plurality of nanoparticles. Dynamic light scattering (DLS) is used to measure D90 values.

The terms “disease”, “disorder”, and “condition” can be used interchangeably, unless indicated otherwise.

As used herein, the term “encapsulation efficiency” (i.e., “EE”) refers to the percentage of payload encapsulated by a plurality of nanoparticles. The encapsulation efficiency is calculated as follows: EE %=(Wₜ/Wᵢ)×100%, where Wₜ is the total amount of payload encapsulated by the plurality of nanoparticles and Wᵢ is the total amount of payload added during preparation of the plurality of nanoparticles.

As used herein, the term “effective amount” refers to an amount of the pharmaceutical composition (e.g., pharmaceutical compositions comprising a plurality of nanoparticles as described herein) that is sufficient to induce a disclosed effect, such as detection or treatment of a disease (e.g., cancer). Therapeutically effective amounts of the pharmaceutical compositions provided herein, will vary depending on the physical properties of the nanoparticles described herein; the activity of the payload; the subject (e.g., the weight and age of the subject); the disease being treated (e.g., cancer); the severity of the disease; the manner of administration of the pharmaceutical composition; and the like.

Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure also includes embodiments in which more than one, or all of the group members are present in, employed in, or otherwise relevant to a given product or process.

The terms “patient” and “subject” can be used interchangeably, unless indicated otherwise, and refer to an animal (e.g., human) for whom diagnosis, treatment, or therapy is desired. A subject may be a pediatric subject (e.g., infant, child, or adolescent) or an adult subject (e.g., young adult, middle-aged adult, or senior adult).

“Percent identity” and “% identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y.

Generally, computer programs are employed for such calculations. Exemplary programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 Apr; 85(8):2444-8; Pearson, Methods Enzymol. 1990; 183:63-98), gapped BLAST (Altschul et al., Nucleic Acids Res. 1997 Sep. 1; 25(17): 3389-40), BLASTP, BLASTN, or GCG (Devereux et al., Nucleic Acids Res. 1984 Jan. 11; 12(1 Pt 1):387-95).

The term “pharmaceutically acceptable salt” refers to a salt of a compound of the present disclosure, wherein the salt is suitable for use in contact with a subject (e.g., human) without undue toxicity, irritation, or allergic response. Pharmaceutically acceptable salts of the compounds of this disclosure include those derived from suitable inorganic and organic acids and bases. Pharmaceutically acceptable salts are well known in the art. For example, S. M. Berge, et al. describes pharmaceutically acceptable salts in detail in J. Pharmaceutical Sciences, 66: 1-19 (1977).

As used herein, the term “polydispersity index” (i.e., “PDI”) refers to a value describing the degree of non-uniformity of a distribution of particle sizes for a plurality of nanoparticles. In some embodiments, the particle size distribution of a plurality of nanoparticles is defined by the polydispersity index. Polydispersity index is calculated as the square of the standard deviation divided by average particle diameter.

As used herein, the term “therapeutically effective amount” generally refers to an amount of a disclosed pharmaceutical composition effective to “treat” a disease in a subject. In some embodiments, a pharmaceutical composition described herein is administered to a subject in an amount that is effective for producing desired therapeutic effect by inhibiting a disease (e.g., cancer) at a reasonable benefit/risk ratio applicable to any medical treatment. A therapeutically effective amount is an amount that achieves at least partially a desired therapeutic or prophylactic effect in an organ or tissue. The amount of a pharmaceutical composition as described herein to bring about prevention or therapeutic treatment of a disease is not fixed. In some embodiments, the amount of the pharmaceutical composition as described herein administered varies with the type of disease, extensiveness of the disease, and size of the subject suffering from the disease. For therapeutic methods involving administration of a therapeutic agent (e.g., nanoparticles comprising a therapeutic agent as described herein) after the subject presents symptoms of a disease, the term “therapeutically effective” means that, after treatment, one or more signs or symptoms of the disease is ameliorated or eliminated.

The terms “treat”, “treating”, and “treatment” refer to an effect that results in the improvement (e.g., lessening, reducing, eliminating, modulating, alleviating, or abating) of a condition, disease, or disorder (e.g., cancer), or symptom(s) thereof.

### BRIEF DESCRIPTION OF THE DRAWINGS

FIG. 1 is an exemplary micelle comprised of conjugate subunits. Each conjugate subunit consists of one hydrophilic 5A peptide headgroup conjugated to a hydrophobic fatty acid. In this example, the fatty acid is myristoyl. A hydrophobic payload is encapsulated in the hydrophobic region in the interior of the micelle formed by the fatty acid domains of the conjugate subunits.

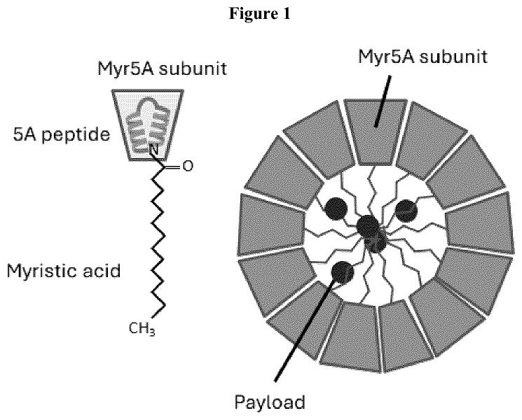

FIG. 2 is a transmission electron microscope image of Myr5A micelle nanoparticles. Image shows multiple spherical nanoparticles of similar size.

FIG. 3 is a high magnification transmission electron microscope image of the microstructure of Myr5A nanoparticles. Image shows that the nanoparticles include an electropaque layer and an inner core constituting the monolayer formed by Myr5A and the internal payload, respectively.

FIG. 4 is a close-up of one of the nanoparticles shown in the TEM image of FIG. 3. The dark outline 1 is the electropaque layer that surrounds the inner core 2 comprising the internal payload.

### DETAILED DESCRIPTION

Numerous compounds fail to advance as therapeutic agents and diagnostic agents (e.g., imaging agents) because of unwanted or harmful side effects. Many of these unwanted or harmful side effects are often associated with the ineffective delivery of the compounds to desired targets such as specific cells, tissues, or organs. For example, if the permeability of chemotherapeutic agents is unspecific, then toxicity results at undesired delivery sites, including healthy cells, tissues, or organs. Furthermore, in order for a diagnostic agent to be effective in, for example, detecting a disease, the diagnostic agent must be specifically delivered to the intended target.

Some compounds have one or more physical properties, such as hydrophobicity or lipophilicity, that are not conducive to effective delivery to biological targets, let alone tailored delivery to particular targets. Some compounds also have one or more physical properties that lead to poor residence time or concentration at a desired target, which fails to induce a desired therapeutic or diagnostic effect. Other compounds are readily degraded or have poor bioavailability or therapeutic index.

Nanoparticles typically have a particle size of about 1 nm to about 1000 nm and are useful due to their small size. Nanoparticles comprising payloads have garnered interest as tools for targeted delivery of therapeutic agents or diagnostic agents. However, there are a number of difficulties that are encountered when designing and producing nanoparticles for effective and safe delivery of compounds to specific targets. For example, the nanoparticles must have physical properties that are controllable and reproducible. The inability to easily and consistently control the physical properties of nanoparticles hinders the use of nanoparticles for delivering agents to desired targets.

In some embodiments, nanoparticles described herein have a physical characteristic that is effective in methods of detecting or treating a disease in a subject in need thereof. In some embodiments, the nanoparticles have a particle size, uniformity in particle size distribution, or both that is effective in methods of detecting or treating a disease in a subject in need thereof. In some embodiments, the nanoparticles described herein enhance specific delivery or permeability of a payload to a desired target. In some embodiments, the nanoparticles described herein enhance residence time or concentration of a payload at a desired target. In some embodiments, the nanoparticles described herein reduce unwanted to harmful side effects (e.g., toxicity) of a payload. In some embodiments, the nanoparticles described herein are used for controlled release of a payload. In some embodiments, the nanoparticles described herein reduce degradation of a payload. In some embodiments, the nanoparticles described herein improve bioavailability or therapeutic index of a payload.

### Nanoparticles

The nanoparticles described herein have a particle size that is measured by using any suitable techniques known in the art. The particle size of a nanoparticle, in some embodiments, depends on the molar ratio of the payload to the conjugate. In some embodiments, the particle size of a nanoparticle results in improved characteristics.

The pluralities of nanoparticles described herein have a level of impurities, an encapsulation efficiency, a mean particle size, a polydispersity index (PDI), or a D90 value that is measured by using any suitable techniques known in the art. The level of impurities, encapsulation efficiency, mean particle size, polydispersity index (PDI), or D90 value of a plurality of nanoparticles, in some embodiments, results in improved characteristics.

Described herein, in some embodiments, are nanoparticles having a particle size of about 5 nm to about 175 nm, comprising a payload and a conjugate comprising the structure of formula (I):

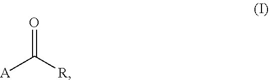

wherein A is a peptide; and R is C₁₋₄₀ alkyl, C₂₋₄₀ alkenyl, or C₂₋₄₀ alkynyl. In some embodiments, the nanoparticles have a payload:conjugate molar ratio of about 1:20 to about 1:1.

Described herein, in some embodiments, are nanoparticles comprising a payload and a conjugate having a payload:conjugate molar ratio of about 1:20 to about 1:1, and wherein the conjugate comprises the structure of formula (I):

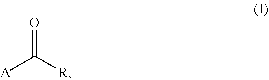

wherein A is a peptide; and R is C₁₋₄₀ alkyl, C₂₋₄₀ alkenyl, or C₂₋₄₀ alkynyl. In some embodiments, the nanoparticles have a particle size of about 5 nm to about 175 nm.

Described herein, in some embodiments, are nanoparticles comprising a payload and a conjugate having a payload:conjugate molar ratio of about 1:20 to about 1:1, and wherein the conjugate comprises the structure of formula (I):

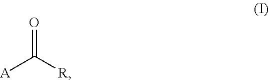

wherein A is a peptide; and R is C₁₋₄₀ alkyl, C₂₋₄₀ alkenyl, or C₂₋₄₀ alkynyl. In some embodiments, the nanoparticles have a particle size of about 175 nm to about 350 nm.

Described herein, in some embodiments, are nanoparticles further comprising an excipient. In some embodiments, the excipient is a lipid. In some embodiments, the lipid is a phospholipid. In some embodiments, the phospholipid is phosphatidylcholine (PC), lysophosphatidylcholine (LPC), phosphatidic acid (PA), lysophosphatidic acid (LPA), phosphatidylethanolamine (PE), lysophosphatidylethanolamine (LPE), phosphatidylglycerol (PG), lysophosphatidylglycerol (LPG), phosphoinositides (PI), lysophosphatidylinositol (LPI), phosphatidylserine (PS), or lysophosphatidylserine (LPS). In some embodiments, the phospholipid is phosphatidylcholine (PC). In some embodiments, the phospholipid is 1,2-dihexanoyl-sn-glycero-3-phosphocholine (DHPC) or 1,2-dimyristoyl-sn-glycero-3-phosphocholine (DMPC). In some embodiments, the phospholipid is 1,2-dihexanoyl-sn-glycero-3-phosphocholine (DHPC). In some embodiments, the phospholipid is 1,2-dimyristoyl-sn-glycero-3-phosphocholine (DMPC). In some embodiments, the lipid is a lipopeptide. In some embodiments, the lipopeptide is a micelle-forming lipopeptide. In some embodiments, the lipopeptide is a palmitoylated peptide. In some embodiments, the lipidated peptide is palmitoyl pentapeptide-4 (Pal-KTTKS(SEQ ID NO: 46)).

Described herein, in some embodiments, are nanoparticles comprising a payload, a conjugate, and a lipid, wherein the conjugate comprises the structure of formula (I):

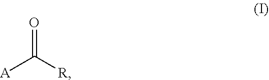

wherein A is a peptide; and R is C₁₋₄₀ alkyl, C₂₋₄₀ alkenyl, or C₂₋₄₀ alkynyl. In some embodiments, the nanoparticles have a particle size of about 5 nm to about 175 nm. In some embodiments, the nanoparticles have a particle size of about 175 nm to about 350 nm.

Described herein, in some embodiments, are nanoparticles comprising a payload, a conjugate, and a lipid, wherein the nanoparticles have a payload:conjugate molar ratio of about 1:20 to about 1:1, wherein the nanoparticles have a payload:lipid molar ratio of about 1:20 to about 1:1, and wherein the conjugate comprises the structure of formula (I):

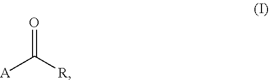

wherein A is a peptide; and R is C₁₋₄₀ alkyl, C₂₋₄₀ alkenyl, or C₂₋₄₀ alkynyl. In some embodiments, the nanoparticles have a particle size of about 5 nm to about 175 nm.

Described herein, in some embodiments, are nanoparticles comprising a payload, a conjugate, and a lipid, wherein the nanoparticles have a payload:conjugate molar ratio of about 1:20 to about 1:1, wherein the nanoparticles have a payload:lipid molar ratio of about 1:20 to about 1:1, and wherein the conjugate comprises the structure of formula (I):

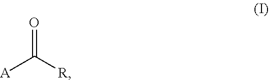

wherein A is a peptide; and R is C₁₋₄₀ alkyl, C₂₋₄₀ alkenyl, or C₂₋₄₀ alkynyl. In some embodiments, the nanoparticles have a particle size of about 175 nm to about 350 nm.

In some embodiments, the particle size is about 5 nm to about 15 nm; about 15 nm to about 25 nm; about 25 nm to about 35 nm; about 35 nm to about 45 nm; about 45 nm to about 55 nm; about 55 nm to about 105 nm; about 55 nm to about 65 nm; about 55 nm to about 85 nm; about 65 nm to about 75 nm; about 75 nm to about 85 nm; about 85 nm to about 95 nm; about 85 nm to about 115 nm; about 95 nm to about 105 nm; about 105 nm to about 175 nm; about 105 nm to about 155 nm; about 105 nm to about 115 nm; about 115 nm to about 125 nm; about 115 nm to about 145 nm; about 125 nm to about 135 nm; about 135 nm to about 145 nm; about 145 nm to about 155 nm; about 145 nm to about 175 nm; about 155 nm to about 165 nm; or about 165 nm to about 175 nm. In some embodiments, the particle size is about 175 nm to about 185 nm; about 185 nm to about 195 nm; about 195 nm to about 205 nm; about 205 nm to about 215 nm; about 215 nm to about 225 nm; about 225 nm to about 235 nm; about 235 nm to about 245 nm; about 245 nm to about 255 nm; about 255 nm to about 265 nm; about 265 nm to about 275 nm; about 275 nm to about 285 nm; about 285 nm to about 295 nm; about 295 nm to about 305 nm; about 305 nm to about 315 nm; about 315 nm to about 325 nm; about 325 nm to about 335 nm; about 335 nm to about 345 nm; about 345 nm to about 350 nm; about 175 nm to about 200 nm; about 200 nm to about 250 nm; about 250 nm to about 300 nm; or about 300 nm to about 350 nm. In some embodiments, the particle size is about 5 nm. In some embodiments, the particle size is about 5 nm. In some embodiments, the particle size is about 5.5 nm. In some embodiments, the particle size is about 6 nm. In some embodiments, the particle size is about 6.5 nm. In some embodiments, the particle size is about 7 nm. In some embodiments, the particle size is about 7.5 nm. In some embodiments, the particle size is about 8 nm. In some embodiments, the particle size is about 8.5 nm. In some embodiments, the particle size is about 9 nm. In some embodiments, the particle size is about 9.5 nm. In some embodiments, the particle size is about 10 nm. In some embodiments, the particle size is about 11 nm. In some embodiments, the particle size is about 12 nm. In some embodiments, the particle size is about 13 nm. In some embodiments, the particle size is about 14 nm. In some embodiments, the particle size is about 15 nm. In some embodiments, the particle size is about 16 nm. In some embodiments, the particle size is about 17 nm. In some embodiments, the particle size is about 18 nm. In some embodiments, the particle size is about 19 nm. In some embodiments, the particle size is about 20 nm. In some embodiments, the particle size is about 22 nm. In some embodiments, the particle size is about 24 nm. In some embodiments, the particle size is about 26 nm. In some embodiments, the particle size is about 28 nm. In some embodiments, the particle size is about 30 nm. In some embodiments, the particle size is about 32 nm. In some embodiments, the particle size is about 34 nm. In some embodiments, the particle size is about 36 nm. In some embodiments, the particle size is about 38 nm. In some embodiments, the particle size is about 40 nm. In some embodiments, the particle size is about 42 nm. In some embodiments, the particle size is about 44 nm. In some embodiments, the particle size is about 46 nm. In some embodiments, the particle size is about 48 nm. In some embodiments, the particle size is about 50 nm. In some embodiments, the particle size is about 55 nm. In some embodiments, the particle size is about 60 nm. In some embodiments, the particle size is about 65 nm. In some embodiments, the particle size is about 70 nm. In some embodiments, the particle size is about 75 nm. In some embodiments, the particle size is about 80 nm. In some embodiments, the particle size is about 85 nm. In some embodiments, the particle size is about 90 nm. In some embodiments, the particle size is about 95 nm. In some embodiments, the particle size is about 100 nm. In some embodiments, the particle size is about 110 nm. In some embodiments, the particle size is about 120 nm. In some embodiments, the particle size is about 130 nm. In some embodiments, the particle size is about 140 nm. In some embodiments, the particle size is about 150 nm. In some embodiments, the particle size is about 160 nm. In some embodiments, the particle size is about 170 nm. In some embodiments, the particle size is about 180 nm. In some embodiments, the particle size is about 190 nm. In some embodiments, the particle size is about 200 nm. In some embodiments, the particle size is about 210 nm. In some embodiments, the particle size is about 220 nm. In some embodiments, the particle size is about 230 nm. In some embodiments, the particle size is about 240 nm. In some embodiments, the particle size is about 250 nm. In some embodiments, the particle size is about 260 nm. In some embodiments, the particle size is about 270 nm. In some embodiments, the particle size is about 280 nm. In some embodiments, the particle size is about 290 nm. In some embodiments, the particle size is about 300 nm. In some embodiments, the particle size is about 310 nm. In some embodiments, the particle size is about 320 nm. In some embodiments, the particle size is about 330 nm. In some embodiments, the particle size is about 340 nm. In some embodiments, the particle size is about 350 nm.

In some embodiments, the payload:conjugate (e.g., Myr5A) molar ratio is about 1:20, about 1:19.5, about 1:19, about 1:18.5, about 1:18, about 1:17.5, about 1:17, about 1:16.5, about 1:16, about 1:15.5, about 1:15, about 1:14.5, about 1:14, about 1:13.5, about 1:13, about 1:12.5, about 1:12, about 1:11.5, about 1:11 about 1:10.5, about 1:10, about 1:9.5, about 1:9, about 1:8.5, about 1:8, about 1:7.5, about 1:7, about 1:6.5, about 1:6, about 1:5.5, about 1:5, about 1:4.5, about 1:4, about 1:3.5, about 1:3, about 1:2.5, about 1:2, about 1:1.5, or about 1:1. In some embodiments, the payload:conjugate molar ratio is about 1:20. In some embodiments, the payload:conjugate molar ratio is about 1:19. In some embodiments, the payload:conjugate molar ratio is about 1:18. In some embodiments, the payload:conjugate molar ratio is about 1:17. In some embodiments, the payload:conjugate molar ratio is about 1:16. In some embodiments, the payload:conjugate molar ratio is about 1:15. In some embodiments, the payload:conjugate molar ratio is about 1:14. In some embodiments, the payload:conjugate molar ratio is about 1:13. In some embodiments, the payload:conjugate molar ratio is about 1:12. In some embodiments, the payload:conjugate molar ratio is about 1:11. In some embodiments, the payload:conjugate molar ratio is about 1:10. In some embodiments, the payload:conjugate molar ratio is about 1:9. In some embodiments, the payload:conjugate molar ratio is about 1:8.5. In some embodiments, the payload:conjugate molar ratio is about 1:8. In some embodiments, the payload:conjugate molar ratio is about 1:7. In some embodiments, the payload:conjugate molar ratio is about 1:6. In some embodiments, the payload:conjugate molar ratio is about 1:5. In some embodiments, the payload:conjugate molar ratio is about 1:4. In some embodiments, the payload:conjugate molar ratio is about 1:3. In some embodiments, the payload:conjugate molar ratio is about 1:2. In some embodiments, the payload:conjugate molar ratio is about 1:1.

In some embodiments, the payload:excipient (e.g., lipid) molar ratio is about 1:20, about 1:19.5, about 1:19, about 1:18.5, about 1:18, about 1:17.5, about 1:17, about 1:16.5, about 1:16, about 1:15.5, about 1:15, about 1:14.5, about 1:14, about 1:13.5, about 1:13, about 1:12.5, about 1:12, about 1:11.5, about 1:11 about 1:10.5, about 1:10, about 1:9.5, about 1:9, about 1:8.5, about 1:8, about 1:7.5, about 1:7, about 1:6.5, about 1:6, about 1:5.5, about 1:5, about 1:4.5, about 1:4, about 1:3.5, about 1:3, about 1:2.5, about 1:2, about 1:1.5, or about 1:1. In some embodiments, the payload:excipient molar ratio is about 1:20. In some embodiments, the payload:excipient molar ratio is about 1:19. In some embodiments, the payload:excipient molar ratio is about 1:18. In some embodiments, the payload:excipient molar ratio is about 1:17. In some embodiments, the payload:excipient molar ratio is about 1:16. In some embodiments, the payload:excipient molar ratio is about 1:15. In some embodiments, the payload:excipient molar ratio is about 1:14. In some embodiments, the payload:excipient molar ratio is about 1:13. In some embodiments, the payload:excipient molar ratio is about 1:12. In some embodiments, the payload:excipient molar ratio is about 1:11. In some embodiments, the payload:excipient molar ratio is about 1:10. In some embodiments, the payload:excipient molar ratio is about 1:9. In some embodiments, the payload:excipient molar ratio is about 1:8.5. In some embodiments, the payload:excipient molar ratio is about 1:8. In some embodiments, the payload:excipient molar ratio is about 1:7. In some embodiments, the payload:excipient molar ratio is about 1:6. In some embodiments, the payload:excipient molar ratio is about 1:5. In some embodiments, the payload:excipient molar ratio is about 1:4. In some embodiments, the payload:excipient molar ratio is about 1:3. In some embodiments, the payload:excipient molar ratio is about 1:2. In some embodiments, the payload:excipient molar ratio is about 1:1.

In some embodiments, the payload:lipid molar ratio is about 1:20, about 1:19.5, about 1:19, about 1:18.5, about 1:18, about 1:17.5, about 1:17, about 1:16.5, about 1:16, about 1:15.5, about 1:15, about 1:14.5, about 1:14, about 1:13.5, about 1:13, about 1:12.5, about 1:12, about 1:11.5, about 1:11 about 1:10.5, about 1:10, about 1:9.5, about 1:9, about 1:8.5, about 1:8, about 1:7.5, about 1:7, about 1:6.5, about 1:6, about 1:5.5, about 1:5, about 1:4.5, about 1:4, about 1:3.5, about 1:3, about 1:2.5, about 1:2, about 1:1.5, or about 1:1. In some embodiments, the payload:lipid molar ratio is about 1:20. In some embodiments, the payload:lipid molar ratio is about 1:19. In some embodiments, the payload:lipid molar ratio is about 1:18. In some embodiments, the payload:lipid molar ratio is about 1:17. In some embodiments, the payload:lipid molar ratio is about 1:16. In some embodiments, the payload:lipid molar ratio is about 1:15. In some embodiments, the payload:lipid molar ratio is about 1:14. In some embodiments, the payload:lipid molar ratio is about 1:13. In some embodiments, the payload:lipid molar ratio is about 1:12. In some embodiments, the payload:lipid molar ratio is about 1:11. In some embodiments, the payload:lipid molar ratio is about 1:10. In some embodiments, the payload:lipid molar ratio is about 1:9. In some embodiments, the payload:lipid molar ratio is about 1:8.5. In some embodiments, the payload:lipid molar ratio is about 1:8. In some embodiments, the payload:lipid molar ratio is about 1:7. In some embodiments, the payload:lipid molar ratio is about 1:6. In some embodiments, the payload:lipid molar ratio is about 1:5. In some embodiments, the payload:lipid molar ratio is about 1:4. In some embodiments, the payload:lipid molar ratio is about 1:3. In some embodiments, the payload:lipid molar ratio is about 1:2. In some embodiments, the payload:lipid molar ratio is about 1:1.

In some embodiments, the conjugate (e.g., Myr5A):excipient (e.g., lipid) molar ratio is about 1:20, about 1:19.5, about 1:19, about 1:18.5, about 1:18, about 1:17.5, about 1:17, about 1:16.5, about 1:16, about 1:15.5, about 1:15, about 1:14.5, about 1:14, about 1:13.5, about 1:13, about 1:12.5, about 1:12, about 1:11.5, about 1:11 about 1:10.5, about 1:10, about 1:9.5, about 1:9, about 1:8.5, about 1:8, about 1:7.5, about 1:7, about 1:6.5, about 1:6, about 1:5.5, about 1:5, about 1:4.5, about 1:4, about 1:3.5, about 1:3, about 1:2.5, about 1:2, about 1:1.5, or about 1:1. In some embodiments, the conjugate:excipient molar ratio is about 1:20. In some embodiments, the conjugate:excipient molar ratio is about 1:19. In some embodiments, the conjugate:excipient molar ratio is about 1:18. In some embodiments, the conjugate:excipient molar ratio is about 1:17. In some embodiments, the conjugate:excipient molar ratio is about 1:16. In some embodiments, the conjugate:excipient molar ratio is about 1:15. In some embodiments, the conjugate:excipient molar ratio is about 1:14. In some embodiments, the conjugate:excipient molar ratio is about 1:13. In some embodiments, the conjugate:excipient molar ratio is about 1:12. In some embodiments, the conjugate:excipient molar ratio is about 1:11. In some embodiments, the conjugate:excipient molar ratio is about 1:10. In some embodiments, the conjugate:excipient molar ratio is about 1:9. In some embodiments, the conjugate:excipient molar ratio is about 1:8.5. In some embodiments, the conjugate:excipient molar ratio is about 1:8. In some embodiments, the conjugate:excipient molar ratio is about 1:7. In some embodiments, the conjugate:excipient molar ratio is about 1:6. In some embodiments, the conjugate:excipient molar ratio is about 1:5. In some embodiments, the payload:lipid molar ratio is about 1:4. In some embodiments, the conjugate:excipient molar ratio is about 1:3. In some embodiments, the conjugate:excipient molar ratio is about 1:2. In some embodiments, the conjugate:excipient molar ratio is about 1:1.

In some embodiments, the payload:(conjugate (e.g., Myr5A)+excipient (e.g., lipid)) molar ratio is about 1:20, about 1:19.5, about 1:19, about 1:18.5, about 1:18, about 1:17.5, about 1:17, about 1:16.5, about 1:16, about 1:15.5, about 1:15, about 1:14.5, about 1:14, about 1:13.5, about 1:13, about 1:12.5, about 1:12, about 1:11.5, about 1:11 about 1:10.5, about 1:10, about 1:9.5, about 1:9, about 1:8.5, about 1:8, about 1:7.5, about 1:7, about 1:6.5, about 1:6, about 1:5.5, about 1:5, about 1:4.5, about 1:4, about 1:3.5, about 1:3, about 1:2.5, about 1:2, about 1:1.5, or about 1:1. In some embodiments, the payload:(conjugate+excipient) molar ratio is about 1:20. In some embodiments, the payload:(conjugate+excipient) molar ratio is about 1:19. In some embodiments, the payload:(conjugate+excipient) molar ratio is about 1:18. In some embodiments, the payload:(conjugate+excipient) molar ratio is about 1:17. In some embodiments, the payload:(conjugate+excipient) molar ratio is about 1:16. In some embodiments, the payload:(conjugate+excipient) molar ratio is about 1:15. In some embodiments, the payload:(conjugate+excipient) molar ratio is about 1:14. In some embodiments, the payload:(conjugate+excipient) molar ratio is about 1:13. In some embodiments, the payload:(conjugate+excipient) molar ratio is about 1:12. In some embodiments, the payload:(conjugate+excipient) molar ratio is about 1:11. In some embodiments, the payload:(conjugate+excipient) molar ratio is about 1:10. In some embodiments, the payload:(conjugate+excipient) molar ratio is about 1:9. In some embodiments, the payload:(conjugate+excipient) molar ratio is about 1:8.5. In some embodiments, the payload:(conjugate+excipient) molar ratio is about 1:8. In some embodiments, the payload:(conjugate+excipient) molar ratio is about 1:7. In some embodiments, the payload:(conjugate+excipient) molar ratio is about 1:6. In some embodiments, the payload:(conjugate+excipient) molar ratio is about 1:5. In some embodiments, the payload:(conjugate+excipient) molar ratio is about 1:4. In some embodiments, the payload:(conjugate+excipient) molar ratio is about 1:3. In some embodiments, the payload:(conjugate+excipient) molar ratio is about 1:2. In some embodiments, the payload:(conjugate+excipient) molar ratio is about 1:1.

In some embodiments, the payload:(conjugate (e.g., Myr5A)+lipid) molar ratio is about 1:20, about 1:19.5, about 1:19, about 1:18.5, about 1:18, about 1:17.5, about 1:17, about 1:16.5, about 1:16, about 1:15.5, about 1:15, about 1:14.5, about 1:14, about 1:13.5, about 1:13, about 1:12.5, about 1:12, about 1:11.5, about 1:11 about 1:10.5, about 1:10, about 1:9.5, about 1:9, about 1:8.5, about 1:8, about 1:7.5, about 1:7, about 1:6.5, about 1:6, about 1:5.5, about 1:5, about 1:4.5, about 1:4, about 1:3.5, about 1:3, about 1:2.5, about 1:2, about 1:1.5, or about 1:1. In some embodiments, the payload:(conjugate+lipid) molar ratio is about 1:20. In some embodiments, the payload:(conjugate+lipid) molar ratio is about 1:19. In some embodiments, the payload:(conjugate+lipid) molar ratio is about 1:18. In some embodiments, the payload:(conjugate+lipid) molar ratio is about 1:17. In some embodiments, the payload:(conjugate+lipid) molar ratio is about 1:16. In some embodiments, the payload:(conjugate+lipid) molar ratio is about 1:15. In some embodiments, the payload:(conjugate+lipid) molar ratio is about 1:14. In some embodiments, the payload:(conjugate+lipid) molar ratio is about 1:13. In some embodiments, the payload:(conjugate+lipid) molar ratio is about 1:12. In some embodiments, the payload:(conjugate+lipid) molar ratio is about 1:11. In some embodiments, the payload:(conjugate+lipid) molar ratio is about 1:10. In some embodiments, the payload:(conjugate+lipid) molar ratio is about 1:9. In some embodiments, the payload:(conjugate+lipid) molar ratio is about 1:8.5. In some embodiments, the payload:(conjugate+lipid) molar ratio is about 1:8. In some embodiments, the payload:(conjugate+lipid) molar ratio is about 1:7. In some embodiments, the payload:(conjugate+lipid) molar ratio is about 1:6. In some embodiments, the payload:(conjugate+lipid) molar ratio is about 1:5. In some embodiments, the payload:(conjugate+lipid) molar ratio is about 1:4. In some embodiments, the payload:(conjugate+lipid) molar ratio is about 1:3. In some embodiments, the payload:(conjugate+lipid) molar ratio is about 1:2. In some embodiments, the payload:(conjugate+lipid) molar ratio is about 1:1.

Described herein, in some embodiments, are pluralities of any nanoparticles described herein. In some embodiments, the pluralities of nanoparticles comprise minimal impurities. As used herein, impurities means raw materials utilized to manufacture the nanoparticles described herein and fragments of such raw materials (e.g., unencapsulated payload, unconjugated peptides, unconjugated fatty acids), peptide-fatty acid conjugates, and empty nanoparticles/micelles (i.e., nanoparticles that do not comprise a payload). In some embodiments, the pluralities of nanoparticles comprise less than about 5% impurities. In some embodiments, the pluralities of nanoparticles comprise less than about 4.5% impurities. In some embodiments, the pluralities of nanoparticles comprise less than about 4% impurities. In some embodiments, the pluralities of nanoparticles comprise less than about 3.5% impurities. In some embodiments, the pluralities of nanoparticles comprise less than about 3% impurities. In some embodiments, the pluralities of nanoparticles comprise less than about 2.5% impurities. In some embodiments, the pluralities of nanoparticles comprise less than about 2% impurities. In some embodiments, the pluralities of nanoparticles comprise less than about 1.5% impurities. In some embodiments, the pluralities of nanoparticles comprise less than about 1% impurities. In some embodiments, the pluralities of nanoparticles comprise less than about 0.5% impurities.

In some embodiments, the pluralities of nanoparticles have a payload encapsulation efficiency of at least about 50%. In some embodiments, the plurality of nanoparticles has a payload encapsulation efficiency of at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%. In some embodiments, the pluralities of nanoparticles have a payload encapsulation efficiency of at least about 50%. In some embodiments, the pluralities of nanoparticles have a payload encapsulation efficiency of at least about 55%. In some embodiments, the pluralities of nanoparticles have a payload encapsulation efficiency of at least about 60%. In some embodiments, the pluralities of nanoparticles have a payload encapsulation efficiency of at least about 65%. In some embodiments, the pluralities of nanoparticles have a payload encapsulation efficiency of at least about 70%. In some embodiments, the pluralities of nanoparticles have a payload encapsulation efficiency of at least about 75%. In some embodiments, the pluralities of nanoparticles have a payload encapsulation efficiency of at least about 80%. In some embodiments, the pluralities of nanoparticles have a payload encapsulation efficiency of at least about 85%. In some embodiments, the pluralities of nanoparticles have a payload encapsulation efficiency of at least about 90%. In some embodiments, the pluralities of nanoparticles have a payload encapsulation efficiency of at least about 95%. In some embodiments, the pluralities of nanoparticles have a payload encapsulation efficiency of at least about 96%. In some embodiments, the pluralities of nanoparticles have a payload encapsulation efficiency of at least about 96.5%. In some embodiments, the pluralities of nanoparticles have a payload encapsulation efficiency of at least about 97%. In some embodiments, the pluralities of nanoparticles have a payload encapsulation efficiency of at least about 97.5%. In some embodiments, the pluralities of nanoparticles have a payload encapsulation efficiency of at least about 98%. In some embodiments, the pluralities of nanoparticles have a payload encapsulation efficiency of at least about 98.5%. In some embodiments, the pluralities of nanoparticles have a payload encapsulation efficiency of at least about 99%. In some embodiments, the pluralities of nanoparticles have a payload encapsulation efficiency of at least about 99.5%.

In some embodiments, the pluralities of nanoparticles have a payload encapsulation efficiency of at least about 25%. In some embodiments, the pluralities of nanoparticles have a payload encapsulation efficiency of at least about 25%. In some embodiments, the pluralities of nanoparticles have a payload encapsulation efficiency of at least about 26%. In some embodiments, the pluralities of nanoparticles have a payload encapsulation efficiency of at least about 27%. In some embodiments, the pluralities of nanoparticles have a payload encapsulation efficiency of at least about 28%. In some embodiments, the pluralities of nanoparticles have a payload encapsulation efficiency of at least about 29%. In some embodiments, the pluralities of nanoparticles have a payload encapsulation efficiency of at least about 30%. In some embodiments, the pluralities of nanoparticles have a payload encapsulation efficiency of at least about 32%. In some embodiments, the pluralities of nanoparticles have a payload encapsulation efficiency of at least about 34%. In some embodiments, the pluralities of nanoparticles have a payload encapsulation efficiency of at least about 36%. In some embodiments, the pluralities of nanoparticles have a payload encapsulation efficiency of at least about 38%. In some embodiments, the pluralities of nanoparticles have a payload encapsulation efficiency of at least about 40%. In some embodiments, the pluralities of nanoparticles have a payload encapsulation efficiency of at least about 42%. In some embodiments, the pluralities of nanoparticles have a payload encapsulation efficiency of at least about 44%. In some embodiments, the pluralities of nanoparticles have a payload encapsulation efficiency of at least about 46%. In some embodiments, the pluralities of nanoparticles have a payload encapsulation efficiency of at least about 48%. In some embodiments, the pluralities of nanoparticles have a payload encapsulation efficiency of at least about 50%.

In some embodiments, the mean particle size of the plurality of nanoparticles is about 5 nm to about 175 nm. In some embodiments, the mean particle size is about 5 nm to about 15 nm; about 15 nm to about 25 nm; about 25 nm to about 35 nm; about 35 nm to about 45 nm; about 45 nm to about 55 nm; about 55 nm to about 105 nm; about 55 nm to about 65 nm; about 55 nm to about 85 nm; about 65 nm to about 75 nm; about 75 nm to about 85 nm; about 85 nm to about 95 nm; about 85 nm to about 115 nm; about 95 nm to about 105 nm; about 105 nm to about 175 nm; about 105 nm to about 155 nm; about 105 nm to about 115 nm; about 115 nm to about 125 nm; about 115 nm to about 145 nm; about 125 nm to about 135 nm; about 135 nm to about 145 nm; about 145 nm to about 155 nm; about 145 nm to about 175 nm; about 155 nm to about 165 nm; or about 165 nm to about 175 nm.

In some embodiments, the polydispersity index of the plurality of nanoparticles is less than about 0.5. In some embodiments, the polydispersity index is less than about 0.45, less than about 0.4, less than about 0.35, less than about 0.3, less than about 0.25, less than about 0.2, less than about 0.15, less than about 0.1, or less than about 0.05. In some embodiments, the polydispersity index is less than about 0.49. In some embodiments, the polydispersity index is less than about 0.48. In some embodiments, the polydispersity index is less than about 0.47. In some embodiments, the polydispersity index is less than about 0.46. In some embodiments, the polydispersity index is less than about 0.45. In some embodiments, the polydispersity index is less than about 0.44. In some embodiments, the polydispersity index is less than about 0.43. In some embodiments, the polydispersity index is less than about 0.42. In some embodiments, the polydispersity index is less than about 0.41. In some embodiments, the polydispersity index is less than about 0.4. In some embodiments, the polydispersity index is less than about 0.39. In some embodiments, the polydispersity index is less than about 0.38. In some embodiments, the polydispersity index is less than about 0.37. In some embodiments, the polydispersity index is less than about 0.36. In some embodiments, the polydispersity index is less than about 0.35. In some embodiments, the polydispersity index is less than about 0.34. In some embodiments, the polydispersity index is less than about 0.33. In some embodiments, the polydispersity index is less than about 0.32. In some embodiments, the polydispersity index is less than about 0.31. In some embodiments, the polydispersity index is less than about 0.3. In some embodiments, the polydispersity index is less than about 0.29. In some embodiments, the polydispersity index is less than about 0.28. In some embodiments, the polydispersity index is less than about 0.27. In some embodiments, the polydispersity index is less than about 0.26. In some embodiments, the polydispersity index is less than about 0.25. In some embodiments, the polydispersity index is less than about 0.24. In some embodiments, the polydispersity index is less than about 0.23. In some embodiments, the polydispersity index is less than about 0.22. In some embodiments, the polydispersity index is less than about 0.21. In some embodiments, the polydispersity index is less than about 0.2. In some embodiments, the polydispersity index is less than about 0.19. In some embodiments, the polydispersity index is less than about 0.18. In some embodiments, the polydispersity index is less than about 0.17. In some embodiments, the polydispersity index is less than about 0.16. In some embodiments, the polydispersity index is less than about 0.15. In some embodiments, the polydispersity index is less than about 0.14. In some embodiments, the polydispersity index is less than about 0.13. In some embodiments, the polydispersity index is less than about 0.12. In some embodiments, the polydispersity index is less than about 0.11. In some embodiments, the polydispersity index is less than about 0.1. In some embodiments, the polydispersity index is less than about 0.09. In some embodiments, the polydispersity index is less than about 0.08. In some embodiments, the polydispersity index is less than about 0.07. In some embodiments, the polydispersity index is less than about 0.06. In some embodiments, the polydispersity index is less than about 0.05. In some embodiments, the polydispersity index is less than about 0.04. In some embodiments, the polydispersity index is less than about 0.03. In some embodiments, the polydispersity index is less than about 0.02. In some embodiments, the polydispersity index is less than about 0.01.

In some embodiments, the mean particle size of the plurality of nanoparticles is about 5 nm to about 175 nm and the polydispersity index is less than about 0.5. In some embodiments, the mean particle size is about 5 nm to about 55 nm; about 5 nm to about 25 nm; of about 25 nm to about 55 nm; about 5 nm to about 15 nm; about 15 nm to about 25 nm; about 25 nm to about 35 nm; about 35 nm to about 45 nm; about 45 nm to about 55 nm; about 55 nm to about 105 nm; about 55 nm to about 65 nm; about 65 nm to about 75 nm; about 75 nm to about 85 nm; about 85 nm to about 95 nm; about 95 nm to about 105 nm; about 105 nm to about 175 nm; about 105 nm to about 155 nm; about 105 nm to about 115 nm; about 115 nm to about 125 nm; about 125 nm to about 135 nm; about 135 nm to about 145 nm; about 145 nm to about 155 nm; about 155 nm to about 165 nm; or about 165 nm to about 175 nm; and the polydispersity index of the plurality of nanoparticles is less than about 0.45, less than about 0.4, less than about 0.35, less than about 0.3, less than about 0.25, less than about 0.2, less than about 0.15, less than about 0.1, or less than about 0.05.

In some embodiments, 90% of the nanoparticles of the plurality of nanoparticles have a particle size less than about 175 nm. In some embodiments, 90% of the nanoparticles of the pluralities of nanoparticles have a particle size less than about 170 nm, less than about 165 nm, less than about 160 nm, less than about 155 nm, less than about 150 nm, less than about 145 nm, less than about 140 nm, less than about 135 nm, less than about 130 nm, less than about 125 nm, less than about 120 nm, less than about 115 nm, less than about 110 nm, less than about 105 nm, less than about 100 nm, less than about 95 nm, less than about 90 nm, less than about 85 nm, less than about 80 nm, less than about 75 nm, less than about 70 nm, less than about 65 nm, less than about 60 nm, less than about 55 nm, less than about 50 nm, less than about 45 nm, less than about 40 nm, less than about 35 nm, less than about 30 nm, less than about 25 nm, less than about 20 nm, less than about 15 nm, or less than about 5 nm. In some embodiments, 90% of the nanoparticles of the plurality of nanoparticles have a particle size less than about 175 nm. In some embodiments, 90% of the nanoparticles of the plurality of nanoparticles have a particle size less than about 170 nm. In some embodiments, 90% of the nanoparticles of the plurality of nanoparticles have a particle size less than about 165 nm. In some embodiments, 90% of the nanoparticles of the plurality of nanoparticles have a particle size less than about 160 nm. In some embodiments, 90% of the nanoparticles of the plurality of nanoparticles have a particle size less than about 155 nm. In some embodiments, 90% of the nanoparticles of the plurality of nanoparticles have a particle size less than about 150 nm. In some embodiments, 90% of the nanoparticles of the plurality of nanoparticles have a particle size less than about 145 nm. In some embodiments, 90% of the nanoparticles of the plurality of nanoparticles have a particle size less than about 140 nm. In some embodiments, 90% of the nanoparticles of the plurality of nanoparticles have a particle size less than about 135 nm. In some embodiments, 90% of the nanoparticles of the plurality of nanoparticles have a particle size less than about 130 nm. In some embodiments, 90% of the nanoparticles of the plurality of nanoparticles have a particle size less than about 125 nm. In some embodiments, 90% of the nanoparticles of the plurality of nanoparticles have a particle size less than about 120 nm. In some embodiments, 90% of the nanoparticles of the plurality of nanoparticles have a particle size less than about 115 nm. In some embodiments, 90% of the nanoparticles of the plurality of nanoparticles have a particle size less than about 110 nm. In some embodiments, 90% of the nanoparticles of the plurality of nanoparticles have a particle size less than about 105 nm. In some embodiments, 90% of the nanoparticles of the plurality of nanoparticles have a particle size less than about 100 nm. In some embodiments, 90% of the nanoparticles of the plurality of nanoparticles have a particle size less than about 95 nm. In some embodiments, 90% of the nanoparticles of the plurality of nanoparticles have a particle size less than about 90 nm. In some embodiments, 90% of the nanoparticles of the plurality of nanoparticles have a particle size less than about 85 nm. In some embodiments, 90% of the nanoparticles of the plurality of nanoparticles have a particle size less than about 80 nm. In some embodiments, 90% of the nanoparticles of the plurality of nanoparticles have a particle size less than about 75 nm. In some embodiments, 90% of the nanoparticles of the plurality of nanoparticles have a particle size less than about 70 nm. In some embodiments, 90% of the nanoparticles of the plurality of nanoparticles have a particle size less than about 65 nm. In some embodiments, 90% of the nanoparticles of the plurality of nanoparticles have a particle size less than about 60 nm. In some embodiments, 90% of the nanoparticles of the plurality of nanoparticles have a particle size less than about 55 nm. In some embodiments, 90% of the nanoparticles of the plurality of nanoparticles have a particle size less than about 50 nm. In some embodiments, 90% of the nanoparticles of the plurality of nanoparticles have a particle size less than about 48 nm. In some embodiments, 90% of the nanoparticles of the plurality of nanoparticles have a particle size less than about 46 nm. In some embodiments, 90% of the nanoparticles of the plurality of nanoparticles have a particle size less than about 44 nm. In some embodiments, 90% of the nanoparticles of the plurality of nanoparticles have a particle size less than about 42 nm. In some embodiments, 90% of the nanoparticles of the plurality of nanoparticles have a particle size less than about 40 nm. In some embodiments, 90% of the nanoparticles of the plurality of nanoparticles have a particle size less than about 38 nm. In some embodiments, 90% of the nanoparticles of the plurality of nanoparticles have a particle size less than about 36 nm. In some embodiments, 90% of the nanoparticles of the plurality of nanoparticles have a particle size less than about 34 nm. In some embodiments, 90% of the nanoparticles of the plurality of nanoparticles have a particle size less than about 32 nm. In some embodiments, 90% of the nanoparticles of the plurality of nanoparticles have a particle size less than about 30 nm. In some embodiments, 90% of the nanoparticles of the plurality of nanoparticles have a particle size less than about 28 nm. In some embodiments, 90% of the nanoparticles of the plurality of nanoparticles have a particle size less than about 26 nm. In some embodiments, 90% of the nanoparticles of the plurality of nanoparticles have a particle size less than about 24 nm. In some embodiments, 90% of the nanoparticles of the plurality of nanoparticles have a particle size less than about 22 nm. In some embodiments, 90% of the nanoparticles of the plurality of nanoparticles have a particle size less than about 20 nm. In some embodiments, 90% of the nanoparticles of the plurality of nanoparticles have a particle size less than about 19 nm. In some embodiments, 90% of the nanoparticles of the plurality of nanoparticles have a particle size less than about 18 nm. In some embodiments, 90% of the nanoparticles of the plurality of nanoparticles have a particle size less than about 17 nm. In some embodiments, 90% of the nanoparticles of the plurality of nanoparticles have a particle size less than about 16 nm. In some embodiments, 90% of the nanoparticles of the plurality of nanoparticles have a particle size less than about 15 nm. In some embodiments, 90% of the nanoparticles of the plurality of nanoparticles have a particle size less than about 14 nm. In some embodiments, 90% of the nanoparticles of the plurality of nanoparticles have a particle size less than about 13 nm. In some embodiments, 90% of the nanoparticles of the plurality of nanoparticles have a particle size less than about 12 nm. In some embodiments, 90% of the nanoparticles of the plurality of nanoparticles have a particle size less than about 11 nm. In some embodiments, 90% of the nanoparticles of the plurality of nanoparticles have a particle size less than about 10 nm. In some embodiments, 90% of the nanoparticles of the plurality of nanoparticles have a particle size less than about 9.5 nm. In some embodiments, 90% of the nanoparticles of the plurality of nanoparticles have a particle size less than about 9 nm. In some embodiments, 90% of the nanoparticles of the plurality of nanoparticles have a particle size less than about 8.5 nm. In some embodiments, 90% of the nanoparticles of the plurality of nanoparticles have a particle size less than about 8 nm. In some embodiments, 90% of the nanoparticles of the plurality of nanoparticles have a particle size less than about 7.5 nm. In some embodiments, 90% of the nanoparticles of the plurality of nanoparticles have a particle size less than about 7 nm. In some embodiments, 90% of the nanoparticles of the plurality of nanoparticles have a particle size less than about 6.5 nm. In some embodiments, 90% of the nanoparticles of the plurality of nanoparticles have a particle size less than about 6 nm. In some embodiments, 90% of the nanoparticles of the plurality of nanoparticles have a particle size less than about 5.5 nm. In some embodiments, 90% of the nanoparticles of the plurality of nanoparticles have a particle size less than about 5 nm.

In some embodiments, the mean particle size is about 5 nm to about 55 nm; about 5 nm to about 25 nm; of about 25 nm to about 55 nm; about 5 nm to about 15 nm; about 15 nm to about 25 nm; about 25 nm to about 35 nm; about 35 nm to about 45 nm; about 45 nm to about 55 nm; about 55 nm to about 105 nm; about 55 nm to about 65 nm; about 65 nm to about 75 nm; about 75 nm to about 85 nm; about 85 nm to about 95 nm; about 95 nm to about 105 nm; about 105 nm to about 175 nm; about 105 nm to about 155 nm; about 105 nm to about 115 nm; about 115 nm to about 125 nm; about 125 nm to about 135 nm; about 135 nm to about 145 nm; about 145 nm to about 155 nm; about 155 nm to about 165 nm; or about 165 nm to about 175 nm; and 90% of the plurality of nanoparticles have a particle size less than 170 nm, less than 165 nm, less than 160 nm, less than 155 nm, less than 150 nm, less than 145 nm, less than 140 nm, less than 135 nm, less than 130 nm, less than 125 nm, less than 120 nm, less than 115 nm, less than 110 nm, less than 105 nm, less than 100 nm, less than 95 nm, less than 90 nm, less than 85 nm, less than 80 nm, less than 75 nm, less than 70 nm, less than 65 nm, less than 60 nm, less than 55 nm, less than 50 nm, less than 45 nm, less than 40 nm, less than 35 nm, less than 30 nm, less than 25 nm, less than 20 nm, less than 15 nm, or less than 5 nm.

In some embodiments, the polydispersity index is less than about 0.45, less than about 0.4, less than about 0.35, less than about 0.3, less than about 0.25, less than about 0.2, less than about 0.15, less than about 0.1, or less than about 0.05; and 90% of the plurality of nanoparticles have a particle size less than 170 nm, less than 165 nm, less than 160 nm, less than 155 nm, less than 150 nm, less than 145 nm, less than 140 nm, less than 135 nm, less than 130 nm, less than 125 nm, less than 120 nm, less than 115 nm, less than 110 nm, less than 105 nm, less than 100 nm, less than 95 nm, less than 90 nm, less than 85 nm, less than 80 nm, less than 75 nm, less than 70 nm, less than 65 nm, less than 60 nm, less than 55 nm, less than 50 nm, less than 45 nm, less than 40 nm, less than 35 nm, less than 30 nm, less than 25 nm, less than 20 nm, less than 15 nm, or less than 5 nm.

In some embodiments, the mean particle size is about 5 nm to about 55 nm; about 5 nm to about 25 nm; of about 25 nm to about 55 nm; about 5 nm to about 15 nm; about 15 nm to about 25 nm; about 25 nm to about 35 nm; about 35 nm to about 45 nm; about 45 nm to about 55 nm; about 55 nm to about 105 nm; about 55 nm to about 65 nm; about 65 nm to about 75 nm; about 75 nm to about 85 nm; about 85 nm to about 95 nm; about 95 nm to about 105 nm; about 105 nm to about 175 nm; about 105 nm to about 155 nm; about 105 nm to about 115 nm; about 115 nm to about 125 nm; about 125 nm to about 135 nm; about 135 nm to about 145 nm; about 145 nm to about 155 nm; about 155 nm to about 165 nm; or about 165 nm to about 175 nm); the polydispersity index less than about 0.45, less than about 0.4, less than about 0.35, less than about 0.3, less than about 0.25, less than about 0.2, less than about 0.15, less than about 0.1, or less than about 0.05); and 90% of the plurality of nanoparticles have a particle size less than 170 nm, less than 165 nm, less than 160 nm, less than 155 nm, less than 150 nm, less than 145 nm, less than 140 nm, less than 135 nm, less than 130 nm, less than 125 nm, less than 120 nm, less than 115 nm, less than 110 nm, less than 105 nm, less than 100 nm, less than 95 nm, less than 90 nm, less than 85 nm, less than 80 nm, less than 75 nm, less than 70 nm, less than 65 nm, less than 60 nm, less than 55 nm, less than 50 nm, less than 45 nm, less than 40 nm, less than 35 nm, less than 30 nm, less than 25 nm, less than 20 nm, less than 15 nm, or less than 5 nm.

### Conjugates

Described herein, in some embodiments, are nanoparticles and pluralities of nanoparticles comprising a conjugate. In some embodiments, the conjugate comprises a peptide covalently attached to an aliphatic carbon chain. In some embodiments, the peptide assists with the delivery of the nanoparticle or plurality of nanoparticles to a desired target. In some embodiments, the peptide is a peptide mimetic of a naturally occurring peptide. In some embodiments, the aliphatic carbon chain is saturated. In some embodiments, the aliphatic carbon chain comprises 6-40 carbon atoms.

In some embodiments, the conjugate has the structure of formula (I):

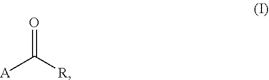

wherein A is a peptide; and R is C₁₋₄₀ alkyl, C₂₋₄₀ alkenyl, or C₂₋₄₀ alkynyl.

In some embodiments, R is C₆₋₄₀ alkyl, C₆₋₄₀ alkenyl, or C₆₋₄₀ alkynyl. In some embodiments, R is C₁₋₂₀ alkyl, C₂₋₂₀ alkenyl, or C₂₋₂₀ alkynyl. In some embodiments, R is C₆₋₂₀ alkyl, C₆₋₂₀ alkenyl, or C₆₋₂₀ alkynyl.

In some embodiments, R is C₁₋₄₀ alkyl. In some embodiments, R is C₆₋₄₀ alkyl. In some embodiments, R is C₁₋₂₀ alkyl. In some embodiments, R is C₆₋₂₀ alkyl.

In some embodiments, R is C₂₋₄₀ alkenyl. In some embodiments, R is C₆₋₄₀ alkenyl. In some embodiments, R is C₂₋₂₀ alkenyl. In some embodiments, R is C₆₋₂₀ alkenyl.

In some embodiments, R is C₂₋₄₀ alkynyl. In some embodiments, R is C₆₋₄₀ alkynyl. In some embodiments, R is C₂₋₂₀ alkynyl. In some embodiments, R is C₆₋₂₀ alkynyl.

In some embodiments, R is:

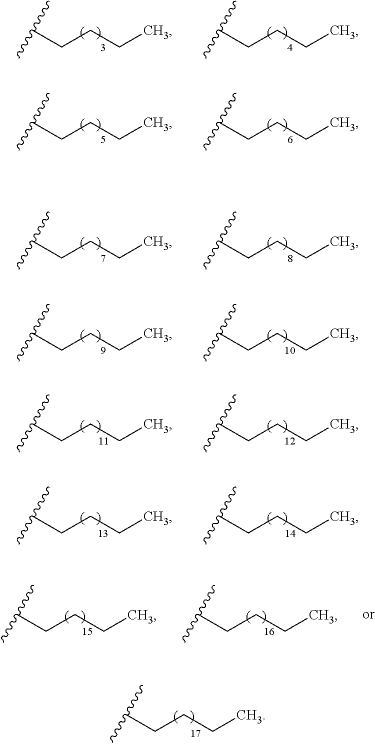

In some embodiments, R is

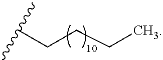

In some embodiments, the conjugate of formula (I) is synthesized by covalently attaching a fatty acid with a peptide. In some embodiments, the fatty acid is enanthic acid, caprylic acid, pelargonic acid, capric acid, undecylic acid, lauric acid, trideclic acid, myristic acid, pentadecylic acid, palmitic acid, margaric acid, stearic acid, nonadecylic acid, arachidic acid, or heneicosylic acid. In some embodiments, the fatty acid is heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecylic acid, eicosanoic acid, or heneicosanoic acid. In some embodiments, the fatty acid is myristic acid.

In some embodiments, the peptide is a linear peptide sequence (i.e., “continuous”). In some embodiments, the peptide comprises a noncontiguous amino acid sequence (i.e., “conformational” or “discontinuous”). In some embodiments, the peptide comprises natural amino acid residues, derivatives of natural amino acid residues, or a combination thereof. In some embodiments, the peptide comprises a residue having an amine group. In some embodiments, the peptide comprises at least 10 residues. In some embodiments, the peptide comprises at least 15 residues, at least 20 residues, at least 25 residues, at least 30 residues, at least 35 residues, at least 40 residues, at least 45 residues, or at least 50 residues.

In some embodiments, the peptide is an apolipoprotein. In some embodiments, the apolipoprotein is apolipoprotein A-I, apolipoprotein A-II, apolipoprotein A-IV, apolipoprotein A-V, apolipoprotein B, apolipoprotein C, apolipoprotein D, or apolipoprotein E. In some embodiments, the apolipoprotein is apolipoprotein A-I.

In some embodiments, the peptide is an apolipoprotein mimetic. In some embodiments, the apolipoprotein mimetic is apolipoprotein A-I mimetic, apolipoprotein A-II mimetic, apolipoprotein A-IV mimetic, apolipoprotein A-V mimetic, apolipoprotein B mimetic, apolipoprotein C mimetic, apolipoprotein D mimetic, or apolipoprotein E mimetic. In some embodiments, the apolipoprotein mimetic is an apolipoprotein A-I mimetic.

In some embodiments, the apolipoprotein A-I mimetic has an amino acid sequence of any one of SEQ ID NOs: 1-45 shown below:

TABLE 1
Apolipoprotein A-I mimetic sequences

| SEQ | |
|---|---|
| ID NO | Sequence |
| 1 | DWLKAFYDKVAEKLKEAFPDWAKAAYDKAAEKAKEAA |
| (5A | |
| peptide) | |
| 2 | DWLKAFYDKVAEKLKEAFPDWLKAFYDKVAEKLKEAF |
| 3 | RMRITERDDFRGQMSEITDDCPSLQDRFHLTEVHSLR |
| | VLEGS |
| 4 | DWLKAFYDKVAEKLKEAFPDWLKAFYDKVAEKAKEAF |
| 5 | DWLKAFYDKVAEKLKEAFPDWLKAFYDKVAEKAKEAA |
| 6 | DWLKAFYDKVAEKLKEAFPDWLKAAYDKVAEKAKEAA |
| 7 | DWLKAFYDKVAEKLKEAFPDWLKAAYDKAAEKAKEAA |
| 8 | DWLKAFYDKVAEKLKEAFPDWGKAGYDKGAEKGKEAG |
| 9 | DWLKAFYDKVAEKLKEAFPDWGKAGYDKGAEKGKEAF |
| 10 | DWGKAGYDKGAEKGKEAGDWLKAFYDKVAEKLKEAF |
| 11 | DWLKAFYDKVAEKLKEAFPDWLKAFYDKVAEKLK |
| 12 | KAFYDKVAEKLKEAFPDWLKAFYDKVAEKLKEAF |
| 13 | DWLKAFYDKVAEKLKEAFPDWLKAFYDKVA |
| 14 | DKVAEKLKEAFPDWLKAFYDKVAEKLKEAF |
| 15 | DWLKAFYDKVAEKLKEAFPDWLKAFYKVAEKLKEAF |
| 16 | DWLKAFYDKVAEKLKEAFPDWLKAFYVAEKLKEAF |
| 17 | DWLAFYDKVAEKLKEAFPDWLKAFYDKVAEKLKEAF |
| 18 | DWLFYDKVAEKLKEAFPDWLKAFYDKVAEKLKEAF |
| 19 | DWLKAFYDKVAEKLKEAFPDWLAKAFYDKVAEKLKEAF |
| 20 | DWLKAFYDKVAEKLKEAFPDWLAAKAFYDKVAEKLKEAF |
| 21 | DWLKAAFYDKVAEKLKEAFPDWLKAFYDKVAEKLKEAF |
| 22 | DWLKAAAFYDKVAEKLKEAFPDWLKAFYDKVAEKLKEAF |
| 23 | DWLKAFYDKVAEKLKEAFPDWLEAFYDKVAKKLKEAF |
| 24 | DWLKAFYDKVAEKLKEAFPDWLEAFYDEVAKKLKKAF |
| 25 | DWLEAFYDKVAKKLKEAFPDWLKAFYDKVAEKLKEAF |
| 26 | DWLEAFYDEVAKKLKKAFPDWLKAFYDKVAEKLKEAF |
| 27 | DWLKAFYDKVAEKLKEAFPDWLKAFYDKVAEKLKEAF |
| 28 | DWLKAFYDKVAEKLKEAFPDWLKAFYDKVAEKLKEAF |
| 29 | DWLKAFYDKVAEKLKEAFPDWLKAFYDKVAEKLKEAF |
| 30 | DWLKAFYDKVAEKLKEAFPDWLKAFYDKVAEKLKEAF |
| 31 | LLDNWDSVTSTFSKLREQPDWAKAAYDKAAEKAKEAA |
| 32 | LESFKVSFLSALEEYTKKPDWAKAAYDKAAEKAKEAA |
| 33 | DWAKAAYDKAAEICAKEAAPLLDNWDSVTSTFSKLREQ |
| 34 | DWAKAAYDKAAEKAKEAAPLESFKVSFLSALEEYTKK |
| 35 | DWLKAFYDKVAEKLKEAFPSDELRQRLAARLEALKEN |
| 36 | DWLKAFYDKVAEKLKEAFPRAELQEGARQKLHELQEK |
| 37 | SDELRQRLAARLEALKENPDWLKAFYDKVAEKLKEAF |
| 38 | RAELQEGARQKLHELQEKPDWLKAFYDKVAEKLKEAF |
| 39 | LLDNWDSVTSTFSKLREQPSDELRQRLAARLEALKEN |
| 40 | LESFKVSFLSALEEYTKKPRAELQEGARQKLHELQEK |
| 41 | SDELRQRLAARLEALKENPLLDNWDSVTSTFSKLREQ |
| 42 | LLDNWDSVTSTFSKLREQPLESFKVSFLSALEEYTKK |
| 43 | DWLKAFYDKVAEKLKEAFPDWLRAFYDKVAEKLKEAF |
| 44 | DWLKAFYDKVAEKLKEAFPDWLRAFYDRVAEKLKEAF |
| 45 | DWLKAFYDKVAEKLKEAFPDWLRAFYDRVAEKLREAF |

In some embodiments, the apolipoprotein A-I mimetic comprises an amino acid sequence having at least about 70% (e.g., 75%, 80%, 90%, 95%, 97%, 98%, or 99%) sequence identity to the amino acid sequence of any one of SEQ ID NOs: 1-45. In some embodiments, the apolipoprotein A-I mimetic comprises an amino acid sequence having at least 75% sequence identity to the amino acid sequence of any one of SEQ ID NOs: 1-45. In some embodiments, the apolipoprotein A-I mimetic comprises an amino acid sequence having at least 80% sequence identity to the amino acid sequence of any one of SEQ ID NOs: 1-45. In some embodiments, the apolipoprotein A-I mimetic comprises an amino acid sequence having at least 85% sequence identity to the amino acid sequence of any one of SEQ ID NOs: 1-45. In some embodiments, the apolipoprotein A-I mimetic comprises an amino acid sequence having at least 90% sequence identity to the amino acid sequence of any one of SEQ ID NOs: 1-45. In some embodiments, the apolipoprotein A-I mimetic comprises an amino acid sequence having at least 95% sequence identity to the amino acid sequence of any one of SEQ ID NOs: 1-45. In some embodiments, the apolipoprotein A-I mimetic comprises an amino acid sequence having at least 96% sequence identity to the amino acid sequence of any one of SEQ ID NOs: 1-45. In some embodiments, the apolipoprotein A-I mimetic comprises an amino acid sequence having at least 97% sequence identity to the amino acid sequence of any one of SEQ ID NOs: 1-45. In some embodiments, the apolipoprotein A-I mimetic comprises an amino acid sequence having at least 98% sequence identity to the amino acid sequence of any one of SEQ ID NOs: 1-45. In some embodiments, the apolipoprotein A-I mimetic comprises an amino acid sequence having at least 99% sequence identity to the amino acid sequence of any one of SEQ ID NOs: 1-45. In some embodiments, the apolipoprotein A-I mimetic comprises an amino acid sequence having the amino acid sequence of any one of SEQ ID NOs: 1-45.

In some embodiments, the apolipoprotein A-I mimetic comprises an amino acid sequence having at least about 70% (e.g., 75%, 80%, 90%, 95%, 97%, 98%, or 99%) sequence identity to the amino acid sequence of SEQ ID NO: 1. In some embodiments, the apolipoprotein A-I mimetic comprises an amino acid sequence having at least 75% sequence identity to the amino acid sequence of SEQ ID NO: 1. In some embodiments, the apolipoprotein A-I mimetic comprises an amino acid sequence having at least 80% sequence identity to the amino acid sequence of SEQ ID NO: 1. In some embodiments, the apolipoprotein A-I mimetic comprises an amino acid sequence having at least 85% sequence identity to the amino acid sequence of SEQ ID NO: 1. In some embodiments, the apolipoprotein A-I mimetic comprises an amino acid sequence having at least 90% sequence identity to the amino acid sequence of SEQ ID NO: 1. In some embodiments, the apolipoprotein A-I mimetic comprises an amino acid sequence having at least 95% sequence identity to the amino acid sequence of SEQ ID NO: 1. In some embodiments, the apolipoprotein A-I mimetic comprises an amino acid sequence having at least 96% sequence identity to the amino acid sequence of SEQ ID NO: 1. In some embodiments, the apolipoprotein A-I mimetic comprises an amino acid sequence having at least 97% sequence identity to the amino acid sequence of SEQ ID NO: 1. In some embodiments, the apolipoprotein A-I mimetic comprises an amino acid sequence having at least 98% sequence identity to the amino acid sequence of SEQ ID NO: 1. In some embodiments, the apolipoprotein A-I mimetic comprises an amino acid sequence having at least 99% sequence identity to the amino acid sequence of SEQ ID NO: 1. In some embodiments, the apolipoprotein A-I mimetic comprises an amino acid sequence having the amino acid sequence of SEQ ID NO: 1.

### Payloads

Described herein, in some embodiments, are nanoparticles and pluralities of nanoparticles comprising a payload. A payload, in some embodiments, is an agent, such as a molecule, that exhibits a particular function or effect. In some embodiments, a payload confers a desired property or effect to a target, or otherwise leads to a desired therapeutic or diagnostic outcome. In some embodiments, a payload is encapsulated in a nanoparticle for delivery to a desired target. Examples of payloads include, but are not limited to, therapeutic agents and diagnostic agents.

Some therapeutic agents or diagnostic agents have a physical property (e.g., hydrophobicity or lipophilicity) that prevent or complicate delivery to a desired target. In some embodiments, the solubility (e.g., in vivo solubility) of the therapeutic agent and diagnostic agent is improved when loaded in a nanoparticle disclosed herein. After encapsulating the hydrophobic or lipophilic payload in a nanoparticle described herein, the solubility, in some embodiments, is improved, allowing for effective delivery of the hydrophobic or lipophilic payload to a desired target.

In some embodiments, the payload is a hydrophobic molecule. In some embodiments, the payload is a lipophilic molecule. Hydrophobicity or lipophilicity can be characterized by the octanol-water partition coefficient. In some embodiments, the payload has an octanol-water partition coefficient greater than 2 (e.g., greater than 2.5, greater than 3, greater than 3.5, greater than 4, greater than 4.5, greater than 5, greater than 5.5, greater than 6, greater than 6.5, greater than 7, greater than 7.5, greater than 8, greater than 8.5, greater than 9, greater than 9.5, or greater than 10).

### Therapeutic Agents

In some embodiments, the payload is a therapeutic agent. In some embodiments, the therapeutic agent is a chemotherapeutic agent. Exemplary chemotherapeutic agents include, but are not limited to, DNA intercalators, mitotic inhibitors, ferroptosis inducers, kinase inhibitors, alkylating agents, topoisomerase inhibitors, microtubule inhibitors, poly adenosine diphosphate ribose polymerase (poly(ADP-ribose) polymerase or PARP) inhibitors, stimulator of interferon genes (STING) agonists, or nucleoside analogs.

Exemplary chemotherapeutic agents include, but are not limited to: cyclophosphamide, chlorambucil, cisplatin, etoposide, ametantrone, bleomycin, dactinomycin, daunorubicin, doxorubicin, epirubicin, idarubicin, mitomycin C, mitoxantrone, N-benzyladriamycin-14-valerate (AD198), valrubicin, docetaxel, monomethyl auristatin E (MMAE), fluorenylmethoxycarbonyl-monomethyl auristatin E (Fmoc-MMAE), monomethyl auristatin F (MMAF), paclitaxel, vinblastine, vincristine, vindesine, vinorelbine, bardoxolone methyl, curcumin, deferasirox, deferoxamine mesylate, erastin, imidazole ketone erastin (IKE), lapatinib, sorafenib, RSL3, ML162, ML210, everolimus, rapamycin, ridaforolimus, sirolimus, temsirolimus, altretamine, bendamustine, busulfan, carboplatin, carmustine, dacarbazine, ifosfamide, lurbinectedin, lomustine, mechlorethamine, melphalan, oxaliplatin, temozolomide, thiotepa, trabectedin, camptothecin, 7-ethyl-10-hydroxycamptothecin (SN-38), exatecan, gimatecan, irinotecan, karenitecin, lurtotecan, rubitecan, silatecan, topotecan, diflomotecan, (S)-13-cyclobutyl-7-ethyl-7-hydroxy-9,12-dihydro-7H-cyclopenta[6,7]indolizino[1,2-b][1,3]dioxolo[4,5-g]quinoline-8,10-dione (S3925), cabazitaxel, eribulin, ixabepilone, tirbanibulin, niraparib, olaparib, pamiparib, rucaparib, talazoparib, veliparib, dimethylxanthone acetic acid (DMXAA or vadimezan), ADU-S100, E7766, MK-1454, acelarin, capecitabine, gemcitabine, sapacitabine, or a pharmaceutically acceptable salt thereof. In some embodiments, the chemotherapeutic agent is IKE. In some embodiments, the chemotherapeutic agent is ML210.

The structure of RSL3, ML162, and ML210 are shown below.

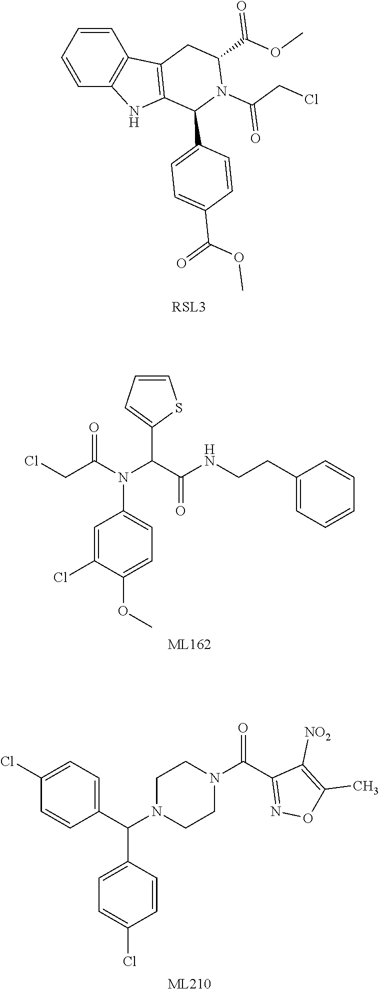 [SMILES: COC(=O)c1ccc([C@H]2c3[nH]c4ccccc4c3C[C@H](C(=O)OC)N2C(=O)CCl)cc1 ; COc1ccc(N(C(=O)CCl)C(C(=O)NCCc2ccccc2)c2cccs2)cc1Cl ; Cc1onc(C(=O)N2CCN(C(c3ccc(Cl)cc3)c3ccc(Cl)cc3)CC2)c1[N+](=O)[O-]]

The structures of ADU-S100, E7766, and MK-1454 are shown below.

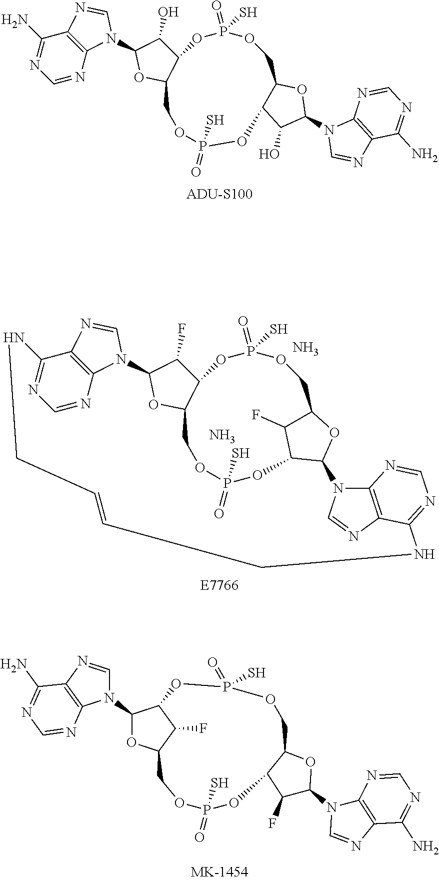

A person of skill in the art would understand that any reference to IR780 (i.e., IR-780 or IR 780), whether in the present application or a prior related application, corresponds to the compound having the following structure:

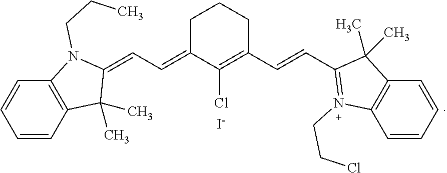

The structure of MMAE is shown below:

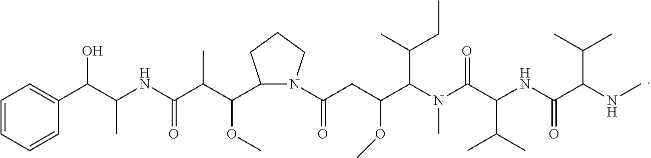 [SMILES: CCC(C)C(C(CC(=O)N1CCCC1C(OC)C(C)C(=O)NC(C)C(O)c1ccccc1)OC)N(C)C(=O)C(NC(=O)C(NC)C(C)C)C(C)C]

The structure of IKE is shown below:

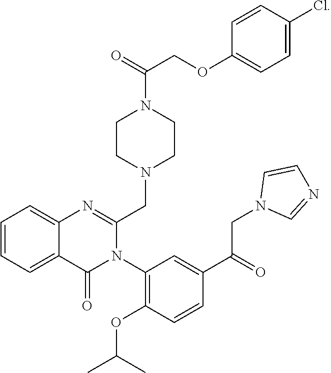 [SMILES: CC(C)Oc1ccc(C(=O)Cn2ccnc2)cc1-n1c(CN2CCN(C(=O)COc3ccc(Cl)cc3)CC2)nc2ccccc2c1=O]

The structure of ML210 (i.e., ML 210 or ML-210) is shown below:

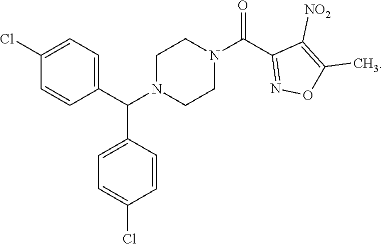 [SMILES: Cc1onc(C(=O)N2CCN(C(c3ccc(Cl)cc3)c3ccc(Cl)cc3)CC2)c1[N+](=O)[O-]]

The structure of Fmoc-MMAE is shown below:

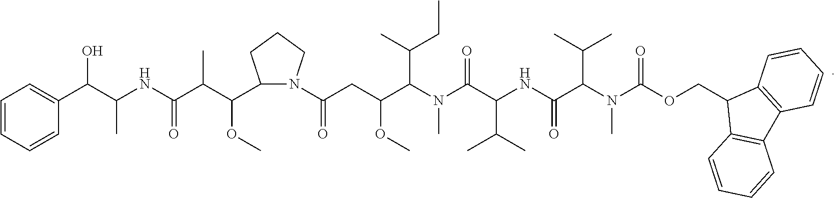 [SMILES: CCC(C)C(C(CC(=O)N1CCCC1C(OC)C(C)C(=O)NC(C)C(O)c1ccccc1)OC)N(C)C(=O)C(NC(=O)C(C(C)C)N(C)C(=O)OCC1c2ccccc2-c2ccccc21)C(C)C]

The structure of AD198 is shown below:

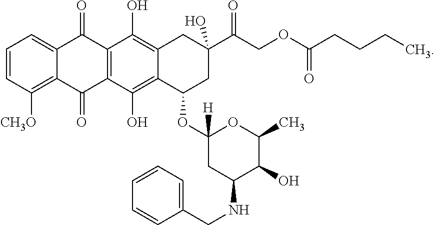

In some embodiments, the therapeutic agent is selected from the group consisting of N-benzyladriamycin-14-valerate (AD198), monomethyl auristatin E (MMAE), fluorenylmethoxycarbonyl-monomethyl auristatin E (Fmoc-MMAE), erastin, imidazole ketone erastin (IKE), rapamycin, lurbinectedin, trabectedin, irinotecan, 7-ethyl-10-hydroxycamptothecin (SN-38), paclitaxel, vincristine, rucaparib, dimethylxanthone acetic acid (DMXAA or vadimezan), acelarin, ML210, or a pharmaceutically acceptable salt thereof.

In some embodiments, the therapeutic agent is N-benzyladriamycin-14-valerate (AD198). In some embodiments, the therapeutic agent is monomethyl auristatin E (MMAE). In some embodiments, the therapeutic agent is fluorenylmethoxycarbonyl-monomethyl auristatin E (Fmoc-MMAE). In some embodiments, the therapeutic agent is erastin. In some embodiments, the therapeutic agent is imidazole ketone erastin (IKE). In some embodiments, the therapeutic agent is rapamycin. In some embodiments, the therapeutic agent is lurbinectedin. In some embodiments, the therapeutic agent is trabectedin. In some embodiments, the therapeutic agent is irinotecan. In some embodiments, the therapeutic agent is 7-ethyl-10-hydroxycamptothecin (SN-38). In some embodiments, the therapeutic agent is paclitaxel. In some embodiments, the therapeutic agent is vincristine. In some embodiments, the therapeutic agent is rucaparib. In some embodiments, the therapeutic agent is dimethylxanthone acetic acid (DMXAA or vadimezan). In some embodiments, the therapeutic agent is acelarin. In some embodiments, the therapeutic agent is vincristine. In some embodiments, the therapeutic agent is ML210.

### Diagnostic Agents

In some embodiments, the payload is a diagnostic agent. In some embodiments, the diagnostic agent is an imaging agent. In some embodiments, the imaging agent comprises a fluorescent dye. Exemplary fluorescent dyes include, but are not limited to, xanthenes (e.g., rhodamines, rhodols and fluoresceins, and their derivatives); bimanes; coumarins and their derivatives (e.g., umbelliferone and aminomethyl coumarins); aromatic amines (e.g., dansyl; squarate dyes); benzofurans; fluorescent cyanines (e.g., heptamethines); indocarbocyanines; carbazoles; dicyanomethylene pyranes; polymethine; oxabenzanthrane; xanthene; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earth metal chelate complexes; and derivatives of such dyes. In some embodiments, the fluorescein dye is, but not limited to, 5-carboxyfluorescein, fluorescein-5-isothiocyanate, fluorescein-6-isothiocyanate and 6-carboxyfluorescein. In some embodiments, the rhodamine dye is, but not limited to, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®), and 2-[2-[2-Chloro-3-[(1,3-dihydro-3,3-dimethyl-1-propyl-2H-indol-2-ylidene)ethylidene]-1-cyclohexen-1-yl]ethenyl]-3,3-dimethyl-1-propylindolium iodide (IR780).

In some embodiments, the imaging agent comprises a fluorescent cyanine. In some embodiments, the fluorescent cyanine is a heptamethine dye. In some embodiments, the heptamethine dye is 2-[2-[2-Chloro-3-[(1,3-dihydro-3,3-dimethyl-1-propyl-2H-indol-2-ylidene)ethylidene]-1-cyclohexen-1-yl]ethenyl]-3,3-dimethyl-1-propylindolium iodide (IR780).

In some embodiments, the diagnostic agent is 2-[2-[2-Chloro-3-[(1,3-dihydro-3,3-dimethyl-1-propyl-2H-indol-2-ylidene)ethylidene]-1-cyclohexen-1-yl]ethenyl]-3,3-dimethyl-1-propylindolium iodide (IR780).

Fluorescent dyes are detected by any suitable method. For example, a fluorescent label is detected by exciting the fluorochrome with the appropriate wavelength of light and detecting the resulting fluorescence, e.g., by microscopy, visual inspection, via photographic film, by the use of electronic detectors such as charge coupled devices (CCDs), or photomultipliers.

### Pharmaceutical Compositions

Described herein, in certain embodiments, are pharmaceutical compositions comprising any of the nanoparticles or pluralities of nanoparticles described herein and a pharmaceutically acceptable carrier, diluent, or excipient. Such compositions are useful for in vitro and in vivo analysis or, in the case of pharmaceutical compositions, for administration to a subject in vivo or ex vivo for imaging or treating a subject with a disease (e.g., cancer).

In some embodiments, the carrier, diluent, or excipient is a adjuvant, antimicrobial agent, antioxidant, buffer, coating, dispersing agent, disintegrant, dye, emulsifier, filler, glidant, lubricant, preservative, salt, solvent, stabilizer, surfactant, suspending agent, tonicity or osmolarity adjusting agent, or other suitable materials known to those skilled in the art. Such materials should be non-toxic and should not interfere with the efficacy of the nanoparticle. The precise nature of the carrier or other material will depend on the route of administration.

Acceptable carrier, diluent, or excipient are those that are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as a polyethylene glycol (PEG).

Acceptable carriers are physiologically acceptable to the administered subject and retain the therapeutic properties of the nanoparticles with/in which it is administered. Acceptable carriers and their formulations are and generally described in, e.g., Remington's Pharmaceutical Sciences, supra. One exemplary carrier is physiological saline. The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject nanoparticles from the administration site of one organ, or portion of the body, to another organ, or portion of the body, or in an in vitro assay system. Each carrier is acceptable in the sense of being compatible with the other ingredients of the formulation and not injurious to a subject to whom it is administered. Nor should an acceptable carrier alter the specific activity of the subject nanoparticles.

In some embodiments, pharmaceutical compositions disclosed herein further comprises an acceptable additive to improve the stability of the nanoparticles in composition and/or to control the release rate of the composition. Acceptable additives do not alter the specific activity of the subject nanoparticles. Exemplary acceptable additives include, but are not limited to, a sugar such as mannitol, sorbitol, glucose, xylitol, trehalose, sorbose, sucrose, galactose, dextran, dextrose, fructose, lactose, and mixtures thereof. Acceptable additives are combined with acceptable carriers and/or excipients such as dextrose, in some embodiments. Alternatively, exemplary acceptable additives include, but are not limited to, a surfactant such as polysorbate 20 or polysorbate 80 to increase stability of the peptide and decrease gelling of the solution. In some embodiments, the surfactant is added to the composition in an amount of 0.01% to 5% of the solution. Addition of such acceptable additives increases the stability and half-life of the composition in storage.

In some embodiments, pharmaceutical compositions disclosed herein are sterile. In some embodiments, a pharmaceutical composition disclosed herein is sterilized by conventional, well known sterilization techniques. For example, sterilization is readily accomplished by filtration through sterile filtration membranes. In some embodiments, the resulting solutions is packaged for use or filtered under aseptic conditions and lyophilized, the lyophilized preparation being combined with a sterile solution prior to administration.

Freeze-drying is employed to stabilize polypeptides for long-term storage, such as when a polypeptide is relatively unstable in liquid compositions, in some embodiments.

In some embodiments, excipients such as, e.g., polyols (including mannitol, sorbitol, and glycerol), sugars (including glucose and sucrose), and amino acids (including alanine, glycine, and glutamic acid) act as stabilizers for freeze-dried products. Polyols and sugars are also used to protect polypeptides from freezing and drying-induced damage and to enhance the stability during storage in the dried state in some embodiments. Sugars are, in some embodiments, effective in both the freeze-drying process and during storage. Other classes of molecules, including mono- and disaccharides and polymers, such as PVP, have also been reported as stabilizers of lyophilized products.

For injection, in some embodiments, a pharmaceutical composition disclosed herein is a powder suitable for reconstitution with an appropriate solution as described above. Examples of these include, but are not limited to, freeze dried, rotary dried, or spray dried powders, amorphous powders, granules, precipitates, or particulates. For injection, the compositions optionally contain stabilizers, pH modifiers, surfactants, bioavailability modifiers, and combinations of these.

In some embodiments, a pharmaceutical composition disclosed herein is designed to be short-acting, fast-releasing, long-acting, or sustained-releasing as described herein. In some embodiments, pharmaceutical compositions disclosed herein are formulated for controlled release or for slow release.

The pharmaceutical composition is administered, e.g., by injection, including, but not limited to, subcutaneous, intravitreal, intradermal, intravenous, intra-arterial, intraperitoneal, intracerebrospinal, or intramuscular injection. Excipients and carriers for use in formulation of compositions for each type of injection are contemplated herein. The following descriptions are by example only and are not meant to limit the scope of the compositions. Compositions for injection include, but are not limited to, aqueous solutions (wherein water soluble) or dispersions, as well as sterile powders for the extemporaneous preparation of sterile injectable solutions or dispersion. For intravenous administration, suitable carriers include physiological saline, bacteriostatic water, or phosphate buffered saline (PBS). In some embodiments, the carrier is a solvent or dispersion medium containing, e.g., water, ethanol, polyol (e.g., glycerol, propylene glycol, and liquid polyethylene glycol, and the like), and suitable mixtures thereof. Fluidity is maintained, e.g., by the use of a coating such as lecithin, by the maintenance of the required particle size in the case of dispersion and by the use of surfactants. Antibacterial and antifungal agents include, e.g., parabens, chlorobutanol, phenol, ascorbic acid, and thimerosal. Isotonic agents, e.g., sugars, polyalcohols such as mannitol, sorbitol, and sodium chloride are included in the composition in some embodiments. In some embodiments, the resulting solutions are packaged for use as is, or lyophilized; the lyophilized preparation is later combined with a sterile solution prior to administration, in some embodiments. For intravenous injection or injection at the site of affliction, the active ingredient will be in the form of a parenterally acceptable aqueous solution which is pyrogen-free and has suitable pH, isotonicity, and stability. Those of relevant skill in the art are well able to prepare suitable solutions using, e.g., isotonic vehicles such as Sodium Chloride Injection, Ringer's Injection, and Lactated Ringer's Injection. Preservatives, stabilizers, buffers, antioxidants, and/or other additives are included as needed, in some embodiments. Sterile injectable solutions are prepared by incorporating an active ingredient in the required amount in an appropriate solvent with one or a combination of ingredients enumerated above, as required, followed by filtered sterilization, in some embodiments. Generally, dispersions are prepared by incorporating the active ingredient into a sterile vehicle which contains a basic dispersion medium and the required other ingredients from those enumerated above. In the case of sterile powders for the preparation of sterile injectable solutions, the preferred methods of preparation are vacuum drying and freeze drying which yields a powder of the active ingredient plus any additional desired ingredient from a previously sterile-filtered solution thereof.

In some embodiments, the pharmaceutical composition is administered intramuscularly, intravenously, intradermally, intranasally, peritoneally, subcutaneously, locally (e.g., by injection or by infusion), mucosally, orally, or topically. Depending on the route of administration, the pharmaceutical composition may be in the form of a capsule, aerosol, cream, emulsion, gel, pill, powder or solid (e.g., lyophilized), solution, suspension, or tablet.

Compositions are administered subcutaneously, in some embodiments, such as by injection of a unit dose. For injection, in some embodiments, an active ingredient is in the form of a parenterally acceptable aqueous solution which is substantially pyrogen-free and has suitable pH, isotonicity, and stability. In some embodiments, one prepares suitable solutions using, e.g., isotonic vehicles such as Sodium Chloride Injection, Ringer's Injection, Lactated Ringer's Injection. Preservatives, stabilizers, buffers, antioxidants, and/or other additives are included, as required, in some embodiments. Additionally, compositions are administered via aerosolization, in some embodiments.

In some embodiments, a pharmaceutical composition disclosed herein is lyophilized, e.g., to increase shelf-life in storage. When the compositions are considered for use in medicaments or any of the methods provided herein, in some embodiments, it is contemplated that the composition are substantially free of pyrogens such that the composition will not cause an inflammatory reaction or an unsafe allergic reaction when administered to a human subject. Testing compositions for pyrogens and preparing compositions substantially free of pyrogens are well understood to one or ordinary skill of the art and are accomplished using commercially available kits in some embodiments.

In some embodiments, acceptable carriers contain a compound that stabilizes, increases, or delays absorption or clearance. Such compounds include, e.g., carbohydrates, such as glucose, sucrose, or dextrans; low molecular weight proteins; compositions that reduce the clearance or hydrolysis of peptides; or excipients or other stabilizers and/or buffers. Agents that delay absorption include, e.g., aluminum monostearate and gelatin. In some embodiments, detergents also be used to stabilize or to increase or decrease the absorption of the pharmaceutical composition, including liposomal carriers. To protect from digestion the compound, in some embodiments, is complexed with a composition to render it resistant to acidic and enzymatic hydrolysis, or the compound is, in some embodiments, complexed in an appropriately resistant carrier such as a liposome. Means of protecting compounds from digestion are known in the art.

### Methods of Use

Disclosed herein, in certain embodiments, are methods of treating a disease or disorder in a subject in need thereof, comprising administering to the subject a pharmaceutical composition comprising (a) a plurality of nanoparticles having a particle size of about 5 nm to about 175 nm, comprising (i) a therapeutic payload and (ii) a conjugate comprising the structure of formula (I):

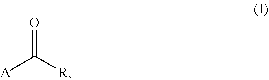

wherein A is a peptide; and R is C₁₋₄₀ alkyl, C₂₋₄₀ alkenyl, or C₂₋₄₀ alkynyl; and (b) a pharmaceutically acceptable excipient. In some embodiments, the therapeutic payload is a hydrophobic therapeutic agent. In some embodiments, the therapeutic agent is a chemotherapeutic agent. In some embodiments, the subject is a pediatric subject.

Disclosed herein, in certain embodiments, are methods of detecting or diagnosing a disease or disorder in a subject in need thereof, comprising (a) administering to the subject a pharmaceutical composition comprising (i) a plurality of nanoparticles having a particle size of about 5 nm to about 175 nm, comprising (A) a diagnostic payload and (B) a conjugate comprising the structure of formula (I):

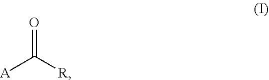

wherein A is a peptide; and R is C₁₋₄₀ alkyl, C₂₋₄₀ alkenyl, or C₂₋₄₀ alkynyl; and (ii) a pharmaceutically acceptable excipient; and (b) detecting the presence of the diagnostic payload. In some embodiments, the subject is a pediatric subject.

In some embodiments of a method disclosed herein, the disease or disorder to be treated and/or diagnosed is a cancer. In some embodiments, the disease or disorder is a rare cancer. In some embodiments, the disease or disorder is a pediatric cancer.

Examples of cancer include, but are not limited to, bile duct cancer, bladder cancer, breast cancer, cervical cancer, colorectal cancer, esophageal cancer, gastrointestinal cancer, liver cancer, nasopharyngeal cancer, ovarian cancer, pancreatic cancer, prostate cancer, adrenocortical carcinoma, lung adenocarcinoma, renal cell carcinoma (e.g., clear cell renal carcinoma), leukemia, lymphoma, neuroblastoma, and sarcoma (e.g., bone sarcoma, soft tissue sarcoma, desmoplastic small round cell tumor (DSRCT), Ewing sarcoma, or rhabdomyosarcoma).

In some embodiments, the cancer is a scavenger receptor class B type 1 (SR-B1) expressing cancer. In some embodiments, the cancer is associated with abnormal (e.g., excessive) expression of scavenger receptor class B type 1 (SR-B1). SR-B1 is a high-density lipoprotein (HDL) receptor that mediates the selective uptake of cholesteryl esters (i.e., esters of cholesterol) and plays an important role in the transfer of cholesterol from HDLs.

In some embodiments, the cancer is associated with abnormal expression of SR-B1. SR-B1 is over-expressed in multiple cancers, including liver cancers, prostate cancers (for example, castration resistant prostate cancer), breast cancers, colorectal cancers, pancreatic cancers, ovarian cancers, and nasopharyngeal cancers. High SR-B1 expression levels are also associated with tumor aggressiveness and poor prognosis. Abnormal expression of SR-B1 is an expression level defined in comparison to a standard reference sample obtained for an individual or a plurality of individuals without a cancer. For example, the abnormal expression may be between about 1-fold to about 1,000 fold greater than the reference sample.

Expression of SR-B1 may be measured by the concentration of the protein produced or expressed from SCARB1. In some embodiments, the concentration SR-B1 is measured by enzyme-linked immunoassay (ELISA), radioimmunoassay, western blotting, or flow cytometry.

Any suitable hydrophobic chemotherapeutic agent may be the therapeutic payload. The particular therapeutic payload will depend on the disease to be treated. In some embodiments, the therapeutic agent is N-benzyladriamycin-14-valerate (AD198), monomethyl auristatin E (MMAE), fluorenylmethoxycarbonyl-monomethyl auristatin E (Fmoc-MMAE), erastin, imidazole ketone erastin (IKE), rapamycin, lurbinectedin, trabectedin, irinotecan, 7-ethyl-10-hydroxycamptothecin (SN-38), paclitaxel, vincristine, rucaparib, dimethylxanthone acetic acid (DMXAA or vadimezan), acelarin, or a pharmaceutically acceptable salt thereof. In some embodiments, the therapeutic agent is N-benzyladriamycin-14-valerate (AD198). In some embodiments, the therapeutic agent is monomethyl auristatin E (MMAE). In some embodiments, the therapeutic agent is fluorenylmethoxycarbonyl-monomethyl auristatin E (Fmoc-MMAE). In some embodiments, the therapeutic agent is erastin. In some embodiments, the therapeutic agent is imidazole ketone erastin (IKE). In some embodiments, the therapeutic agent is rapamycin. In some embodiments, the therapeutic agent is lurbinectedin. In some embodiments, the therapeutic agent is trabectedin. In some embodiments, the therapeutic agent is irinotecan. In some embodiments, the therapeutic agent is 7-ethyl-10-hydroxycamptothecin (SN-38). In some embodiments, the therapeutic agent is paclitaxel. In some embodiments, the therapeutic agent is vincristine. In some embodiments, the therapeutic agent is rucaparib. In some embodiments, the therapeutic agent is dimethylxanthone acetic acid (DMXAA or vadimezan). In some embodiments, the therapeutic agent is acelarin.

Any suitable hydrophobic diagnostic agent may be the diagnostic payload. The particular diagnostic payload will depend on the disease to be detected and/or the tissue of interest. In some embodiments, the diagnostic payload is an imaging agent. In some embodiments, the imaging agent is a fluorescent dye disclosed herein. In some embodiments, the imaging agent is a fluorescent cyanine disclosed herein. In some embodiments, the fluorescent cyanine is 2-[2-[2-Chloro-3-[(1,3-dihydro-3,3-dimethyl-1-propyl-2H-indol-2-ylidene)ethylidene]-1-cyclohexen-1-yl]ethenyl]-3,3-dimethyl-1-propylindolium iodide (IR780). The diagnostic payload is detected by any suitable method. For example, if the diagnostic payload is IR780, then following administration of the nanoparticle, a sample of tissue of interest is excised from the patient and imaged by any suitable means.

In some embodiments, described herein, are methods of treating adrenocortical carcinoma (ACC) in a subject in need thereof comprising administering to the subject nanoparticles described herein comprising imidazole ketone erastin (IKE). In some embodiments, the subject is a pediatric subject (e.g., infant, child, or adolescent). In some embodiments, the subject is an adolescent.

### Routes of Administration

In some embodiments, pharmaceutical compositions disclosed herein are formulated for any suitable route of administration to a subject including, but not limited to, injection (e.g., subcutaneous injection). Injection includes, e.g., subcutaneous, peritoneal, intravenous injection, intramuscular injection, or spinal injection into the cerebrospinal fluid (CSF). In some embodiments, pharmaceutical compositions of the disclosure are formulated for subcutaneous administration. In some embodiments, pharmaceutical compositions of the disclosure are formulated for peritoneal administration. In some embodiments, pharmaceutical compositions of the disclosure are formulated for intravenous administration (e.g., intravenous injection or infusion). In some embodiments, pharmaceutical compositions of the disclosure are formulated for intramuscular administration. In some embodiments, pharmaceutical compositions of the disclosure (of the disclosure are formulated for spinal injection into the cerebrospinal fluid (CSF) administration. In some embodiments, administration is in one, two, three, four, five, six, seven, or more injection sites.

For in vivo applications, contacting occurs, e.g., via administration of a composition described herein to a subject by any suitable means. Pharmaceutical compositions described herein, in some embodiments, are administered, either systemically or locally, including via parenteral, intraperitoneal, intracerebrospinal, intrapulmonary, and intranasal administration, and, if desired for local treatment, intralesional administration. Parenteral routes include, but are not limited to, subcutaneous, intravenous, intraarterial, intraperitoneal, epidural, intramuscular, and intrathecal administration. Such administration, in some embodiments, is as a bolus, continuous infusion, or pulse infusion. In some embodiments, pharmaceutical compositions are administered by injection (e.g., subcutaneous injection) depending in part on whether the administration is brief or chronic. Other modes of administration methods are contemplated, including topical, particularly transdermal, transmucosal, rectal, oral, or local administration e.g., through a catheter placed close to the desired site.

### Dosage

The pharmaceutical compositions described herein are administered in a manner compatible with the dosage formulation and in a therapeutically effective amount. The quantity to be administered depends on the subject to be treated and degree of binding capacity desired. Precise amounts of active ingredient required to be administered depend on the judgment of the practitioner and are peculiar to each subject. Suitable regimes for initial administration and booster shots are also variable, but are typified by an initial administration followed by repeated doses at one hour intervals or longer by a subsequent injection or other administration. Alternatively, continuous intravenous infusion that is sufficient to maintain concentrations in the blood are contemplated.

The amounts of the active ingredients (e.g., nanoparticles of the disclosure) in the pharmaceutical compositions, the pharmaceutical composition formulation, and the mode of administration, are among the factors that are varied to provide an amount of the active ingredient that is effective to achieve the desired therapeutic response for each subject, without being unduly toxic to the subject. The selected dosage level will depend upon a variety of factors including the activity of the particular compound employed, the route of administration, the time of administration, the rate of excretion or metabolism of the particular compound being employed, the duration of the treatment, other drugs, compounds and/or materials used in combination with the particular composition employed, the age, sex, weight, condition, general health, diet and prior medical history of the subject being treated, and like factors well known in the medical arts.

Therapeutically effective amounts of the pharmaceutical compositions vary and depend on the severity of the disease, the subject's weight, and general state of the subject being treated. Administration is, in some embodiments, daily, on alternating days, weekly, twice a month, monthly, or more or less frequently, as necessary depending on the response of the disorder or condition and the subject's tolerance to the therapy. In some embodiments, maintenance dosages over a longer period of time, such as 4, 5, 6, 7, 8, 10, or 12 weeks or longer, are needed until a desired suppression of disorder symptoms occurs, and dosages are adjusted as necessary. The progress of this therapy is easily monitored by conventional techniques and assays.

A physician having ordinary skill in the art, in some cases, readily determines and prescribes the effective amount (ED50) of the composition required. For example, the physician or veterinarian could start doses of the compounds employed in the composition at levels lower than that required to achieve the desired therapeutic effect and gradually increase the dosage until the desired effect is achieved. Alternatively, a dose remains constant in some embodiments.

Toxicity and therapeutic efficacy of the disclosed compositions are, in some embodiments, determined by standard pharmaceutical procedures in cell cultures or experimental animals, e.g., for determining the LD50 (the dose lethal to 50% of the population) and the ED50 (the dose therapeutically effective in 50% of the population). In some embodiments, the dose ratio between toxic and therapeutic effects is the therapeutic index and is expressed as the ratio LD50/ED50. While compounds that exhibit toxic side effects are used in some embodiments, care should be taken to design a delivery system that targets such compounds to the site of affected tissue to minimize potential damage to healthy cells and, thereby, reduce side effects.

Data obtained from cell culture assays and animal studies are used in formulating a range of dosage for use in humans, in some embodiments. The dosage of such compounds lies preferably within a range of circulating concentrations that include the ED50 with little or no toxicity. In some embodiments, the dosage varies within this range depending upon the dosage form employed and the route of administration utilized. In some embodiments, a dose is formulated in animal models to achieve a circulating plasma concentration range that includes the ED50. Levels in plasma are measured, e.g., by high performance liquid chromatography, mass spectrometry, or other routine methods. Such information is, in some cases, used to more accurately determine useful doses in humans.

### Examples

### Example 1. Synthesis of Myr5A Nanoparticles

In this example, Myr5A nanoparticles were synthesized.

Method: First, to a stirring solution of Myr5A (e.g., 10 mg/mL) was added DCM dropwise. The mixture was then heated at 60° C. with stirring for 30 minutes. Then the sample was cooled on ice for 10 minutes. Subsequently, the sample was lyophilized using trehalose (e.g., 10 mg/mL).

The average particle size, polydispersity index (PDI), and D90 of the pre-lyophilized Myr5A nanoparticles and post-lyophilized Myr5A nanoparticles were measured. The results are shown in the table below.

TABLE 2

| | Average particle | | |
|---|---|---|---|
| Nanoparticles | size (nm) | PDI | D90 (nm) |
| Pre-lyophilized Myr5A | 128.2 | 0.189 | 73 |
| nanoparticles | | | |
| Post-lyophilized Myr5A | 88.5 | Multimodal | 71.6 |
| nanoparticles | | | |

### Example 2. Synthesis of AD198 Myr5A Nanoparticles (Direct Dissolution)

In this example, AD198 Myr5A nanoparticles were synthesized using direct dissolution.

Materials: Myr5A, AD198, Dichloromethane (DCM), and ACS 99.5% with amylene.

Method (direct dissolution): A 5 mL tube was charged with lyophilized Myr5A. Water was added to the tube in aliquots to the desired concentration (e.g., 10 mg/mL) and vortexed until dissolved. Higher concentration of Myr5A can also be used. Solution of Myr5A was then placed in a beaker with a stir bar and then placed on the heated stir place set to 60° C. Meanwhile, AD198 was weighed out (1:10 molar ratio of AD198:Myr5A) and dissolved in DCM.

The solution of AD198 in DCM was added to the solution of Myr5A, and the mixture was heated at 50° C. with agitation for 40 minutes. After the heated agitation, the final volume of the mixture was measured. The mixture was removed from stirring and centrifuged at 5000 rpm for 15 minutes. Synthesis of nanoparticles was performed without the use of a microfluidic system.

Results: Using the direct dissolution method described above, 1:3, 1:5, 1:7, 1:10 molar ratio AD198 Myr5A nanoparticles were synthesized.

The average particle size, polydispersity index (PDI), and D90 of the pre-lyophilized and post-lyophilized 1:10 molar ratio AD198 Myr5A nanoparticles were measured. The results are shown in the table below.

TABLE 3

| | Average particle | | D90 |
|---|---|---|---|
| Nanoparticles | size (nm) | PDI | (nm) |
| Pre-lyophilized 1:3 AD198 | 109.3 | 0.226 | 55.9 |
| Myr5A nanoparticles | | | |
| Post-lyophilized 1:3 AD198 | 58.0 | 0.541 | 70.6 |
| Myr5A nanoparticles | | | |
| Pre-lyophilized 1:5 AD198 | 105.3 | 0.139 | 52.8 |
| Myr5A nanoparticles | | | |
| Post-lyophilized 1:5 AD198 | 75.8 | Multimodal | 116 |
| Myr5A nanoparticles | | | |
| Pre-lyophilized 1:7 AD198 | 92.8 | 0.143 | 53 |
| Myr5A nanoparticles | | | |
| Post-lyophilized 1:7 AD198 | 127.5 | Multimodal | 134 |
| Myr5A nanoparticles | | | |
| Pre-lyophilized 1:10 AD198 | 106.6 | 0.192 | 50.7 |
| Myr5A nanoparticles | | | |
| Post-lyophilized 1:10 AD198 | 76.2 | Multimodal | 86.7 |
| Myr5A nanoparticles | | | |

### Example 3. Synthesis of AD198 Myr5A Nanoparticles (Dropwise Addition)

In this example, AD198 Myr5A nanoparticles were synthesized using dropwise addition.

Method: Myr5A was dissolved in water. AD198 was dissolved in DCM. Molar ratio of AD198:Myr5A was 1:10. The solution of AD198 in DCM was added dropwise to the solution of Myr5A. The resulting mixture was left on heated stir plate at 60° C. for 30 minutes. Then the solution was placed on ice for 10 minutes. The leftover solution was lyophilized using trehalose.

Results: The average particle size, polydispersity index (PDI), and D90 of the pre-lyophilized and post-lyophilized 1:10 molar ratio AD198 Myr5A nanoparticles were measured. The results are shown in the table below.

TABLE 4

| | Average particle | | D90 |
|---|---|---|---|
| Nanoparticles | size (nm) | PDI | (nm) |
| Pre-lyophilized 1:10 AD198 | 106.6 | 0.192 | 50.7 |
| Myr5A nanoparticles | | | |
| Post-lyophilized 1:10 AD198 | 76.2 | Multimodal | 86.7 |
| Myr5A nanoparticles | | | |

### Example 4. Synthesis of AD198 Myr5A Nanoparticles (Dropwise Addition)

In this example, a large batch (AD198: 12 mg; Myr5A: 740.28 mg) of 1:10 molar ratio AD198 Myr5A nanoparticles were synthesized using the dropwise addition method described in Example 3.

Pre-lyophilization: The average particle size, polydispersity index (PDI), and D90 of the pre-lyophilized and post-lyophilized 1:10 molar ratio AD198 Myr5A nanoparticles were measured. The results are shown in the table below.

TABLE 5

| | Average particle | | D90 |
|---|---|---|---|
| Nanoparticles | size (nm) | PDI | (nm) |
| Pre-lyophilized 1:10 AD198 | 140 | 0.096 | 74.3 |
| Myr5A nanoparticles | | | |
| Post-lyophilized 1:10 AD198 | 91.5 | Multimodal | 119 |
| Myr5A nanoparticles | | | |

The encapsulation efficiency (EE %) of the post-lyophilized 1:10 AD198 Myr5A nanoparticles was measured by high-performance liquid chromatography (HPLC): 97% at conc. 0.529 mg/mL.

### Example 5. Synthesis of AD198 Myr5A Nanoparticles (Dropwise Addition)

In this example, 1:15 molar ratio AD198 Myr5A nanoparticles were synthesized using the dropwise addition method described in Example 4.

Results: The average particle size, polydispersity index (PDI), and D90 of the pre-lyophilized and post-lyophilized 1:15 molar ratio AD198 Myr5A nanoparticles were measured. The results are shown in the table below.

TABLE 6

| | Average particle | | D90 |
|---|---|---|---|
| Nanoparticles | size (nm) | PDI | (nm) |
| Pre-lyophilized 1:15 AD198 | 105.3 | 0.139 | 52.3 |
| Myr5A nanoparticles | | | |
| Post-lyophilized 1:15 AD198 | 80.7 | Multimodal | 116 |
| Myr5A nanoparticles | | | |

The encapsulation efficiency (EE %) of the post-lyophilized 1:15 AD198 Myr5A nanoparticles was measured by high-performance liquid chromatography (HPLC): 87% at conc. 0.094 mg/mL.

### Example 6. Synthesis of IR780 Myr5A Nanoparticles (Dropwise Addition)

In this example, IR780 Myr5A nanoparticles were synthesized.

Method: Myr5A was dissolved in water. IR780 was dissolved in DCM. The solution of IR780 in DCM was added dropwise to the solution of Myr5A. The resulting mixture was left on heated stir plate at 60° C. for 30 minutes. The solution was then spun down at 5000 rpm for 10 minutes. Subsequently, the solution was placed on ice for 10 minutes. The leftover solution was lyophilized using trehalose.

Results: Using the dropwise method described above, 1:5, 1:7, and 1:10 molar ratio IR780 Myr5A nanoparticles were synthesized.

Pre-lyophilization: The average particle size, polydispersity index (PDI), and D90 of the pre-lyophilized 1:5, 1:7, and 1:10 molar ratio IR780 Myr5A nanoparticles were measured. The results are shown in the table below.

TABLE 7

| Molar ratio of | Average particle | | D90 |
|---|---|---|---|
| IR780:Myr5A | size (nm) | PDI | (nm) |
| 1:5 | 120.9 | 0.230 | 98.3 |
| 1:7 | 93.1 | 0.233 | 50.6 |
| 1:10 | 102.8 | 0.238 | 47.8 |

Post-lyophilization: The average particle size, PDI, and D90 of the post-lyophilized 1:5, 1:7, and 1:10 molar ratio IR780 Myr5A nanoparticles were measured. The results are shown in the table below.

TABLE 8

| Molar | Average | | | Encapsulation |
|---|---|---|---|---|
| ratio of | particle size | | D90 | efficiency |
| IR780:Myr5A | (nm) | PDI | (nm) | (EE) |
| 1:5 | 150.4 | Multimodal | 57.3 | 67.5% at conc. |
| | | | | 0.316 mg/mL |
| 1:7 | 128.5 | 0.175 | 59.5 | 47.8% at conc. |
| | | | | 0.111 mg/mL |
| 1:10 | 172.1 | 0.188 | 107 | 96.2% at conc. |
| | | | | 0.156 mg/mL |

Long-term stability of IR780 Myr5A nanoparticles was determined.

Method: Using the dropwise methods described above, 1:7 molar ratio IR780 Myr5A nanoparticles were synthesized. The average particle size, PDI, and D90 of the 1:7 ratio Myr5A IR780 nanoparticles were measured. The results are shown in the table below.

TABLE 9

| Average particle | | D90 | Encapsulation efficiency |
|---|---|---|---|
| size (nm) | PDI | (nm) | (EE) |
| 154.5 | 0.087 | 67.2 | 55% at conc. 0.130 mg/mL |

Results: After storing the IR780 Myr5A nanoparticles as a lyophilized cake for 94 days, the lyophilized cake was resuspended in water (0.2 mg/mL). The average particle size, polydispersity index (PDI), and D90 of the resuspended IR780 Myr5A nanoparticles were measured. The results are shown in the table below.

TABLE 10

| Average particle | | D90 | |
|---|---|---|---|
| size (nm) | PDI | (nm) | Potency |
| 91.1 | 0.250 | 63.6 | 57% at 0.114 mg/mL |

In the table above, potency is the actual measured concentration of IR780 (measured by HPLC) divided by the expected concentration when the IR780 Myr5A nanoparticles were synthesized.

### Example 7. Synthesis of MMAE Myr5A Nanoparticles (Dropwise Addition)

In this example, MMAE Myr5A nanoparticles were synthesized.

Method: Myr5A was dissolved in water. MMAE was dissolved in DCM. The solution of MMAE in DCM was added dropwise to the solution of Myr5A. The resulting mixture was left on heated stir plate at 60° C. for 30 minutes. Subsequently, the solution was placed on ice for 10 minutes. The leftover solution was lyophilized using trehalose (e.g., 10 mg/mL).

Results: Using the dropwise method described above, 1:3, 1:5, and 1:10 molar ratio MMAE Myr5A nanoparticles were synthesized.

Pre-lyophilization: No pre-lyophilization measurement performed.

Post-lyophilization: The average particle size, PDI, and D90, and Encapsulation efficiency (EE) of the post-lyophilized 1:3, 1:5, and 1:10 molar ratio MMAE Myr5A nanoparticles were measured. The results are shown in the table below.

TABLE 11

| | Average | | | Encapsulation |
|---|---|---|---|---|
| Molar ratio of | particle size | | D90 | efficiency |
| MMAE:Myr5A | (nm) | PDI | (nm) | (EE) |
| 1:3 | 62.2 | 0.332 | 55 | 21.4% at conc. |
| | | | | 0.126 mg/mL |
| 1:5 | 18.1 | Multimodal | 194 | — |
| 1:10 | 7.5 | 0.196 | 7.27 | — |

### Example 8. Solvent Screen of MMAE Myr5A Nanoparticles (Dropwise Addition)

A solvent screen of MMAE was conducted. Acetone, ethanol, and isopropanol showed good solubility of MMAE.

Results: Using the dropwise method described above, 1:7 molar ratio MMAE Myr5A nanoparticles were synthesized using the solvents shown in the table below.

Pre-lyophilization: The average particle size, polydispersity index (PDI), and D90 of the pre-lyophilized 1:7 molar ratio MMAE Myr5A nanoparticles were measured. The results are shown in the table below.

TABLE 12

| | | Average | | |
|---|---|---|---|---|
| Molar | | particle | | |
| ratio of | | size | | D90 |
| MMAE:Myr5A | MMAE Solvent | (nm) | PDI | (nm) |
| 1:7 | Acetone:DCM (1:4) | 143.1 | 0.135 | 78.4 |
| 1:7 | Methanol:DCM (1:4) | 34 | Multimodal | 60 |
| 1:7 | Ethanol:DCM (1:4) | 85.3 | Multimodal | 51.7 |
| 1:7 | ACN:DCM (3:7) | 0.9 | Multimodal | 4100 |
| 1:7 | ACN:DCM (3:7) | 1 | Multimodal | 4680 |
| 1:7 | Ethanol:DCM (3:7) | 1 | Multimodal | 1120 |
| 1:7 | Ethanol:DCM (3:7) | 1.1 | Multimodal | 1070 |
| 1:7 | Methanol:DCM (3:7) | 0.6 | Multimodal | 48.5 |
| 1:7 | Methanol:DCM (3:7) | 1 | Multimodal | 1090 |

Post-lyophilization: The average particle size, PDI, D90, and Encapsulation efficiency (EE) of the post-lyophilized 1:7 molar ratio MMAE Myr5A nanoparticles were measured. The results are shown in the table below.

TABLE 13

| Molar | | Average | | | |
|---|---|---|---|---|---|
| ratio of | MMAE | particle size | | D90 | Encapsulation |
| MMAE:Myr5A | Solvent | (nm) | PDI | (nm) | efficiency (EE) |
| 1:7 | Acetone:DCM (1:4) | 86.2 | 0.294 | 58.1 | 10% at conc. |
| | | | | | 0.383 mg/mL |
| 1:7 | Methanol:DCM (1:4) | 99.8 | Multimodal | 80.8 | 0% at conc. |
| | | | | | 0.442 mg/mL |
| 1:7 | Ethanol:DCM (1:4) | 70.3 | 0.281 | 39 | 0% at conc. |
| | | | | | 0.567 mg/mL |

MMAE Myr5A nanoparticles were synthesized using a reverse dropwise synthetic procedure (solution of Myr5A added to solution of to solution of MMAE).

Results: Using the dropwise method described above, 1:7 molar ratio MMAE Myr5A nanoparticles were synthesized using the solvents shown in the table below.

Pre-lyophilization: The average particle size, polydispersity index (PDI), and D90 of the pre-lyophilized 1:7 molar ratio MMAE Myr5A nanoparticles were measured. The results are shown in the table below.

TABLE 14

| Molar ratio of | | Average particle | | D90 |
|---|---|---|---|---|
| MMAE:Myr5A | MMAE Solvent | size (nm) | PDI | (nm) |
| 1:7 | DCM | 178.3 | 0.116 | 92.3 |

The nanoparticles in the table above also showed an Encapsulation efficiency (EE) of 3% at 0.318 mg/mL.

### Example 9. Transmission Electron Microscope (TEM) Imaging

In this example, Myr5A nanoparticles were synthesized according to the method described in Example 1. Then TEM images were obtained.

Result: Myr5A nanoparticle sample was resuspended in water at a concentration of 8 mg/mL Myr5A and stained using uracyl acetate at a ratio of 1:1. The sample was then placed on a copper plate prior to imaging on TEM. Uracyl acetate tends to provide a contrast agent to visualize the nanoparticles and accumulation of uracyl acetate is not specific.

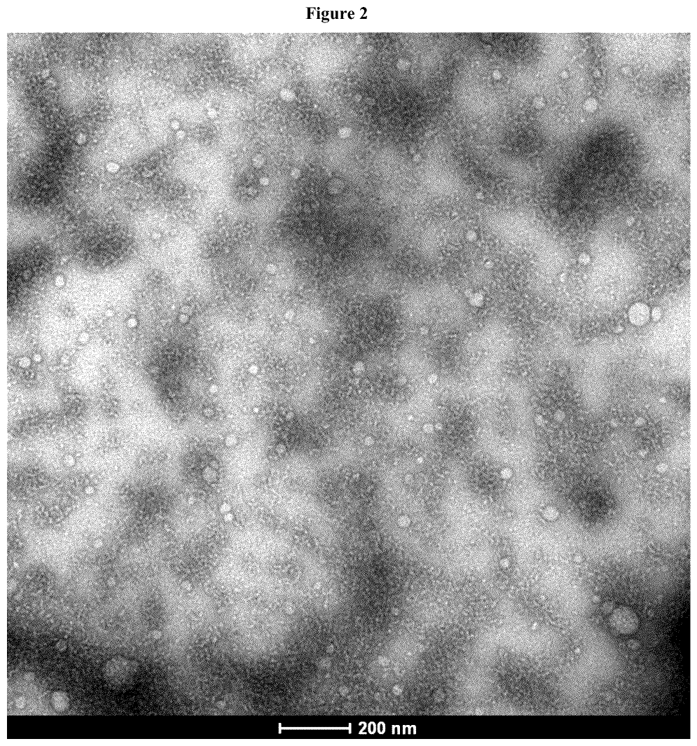

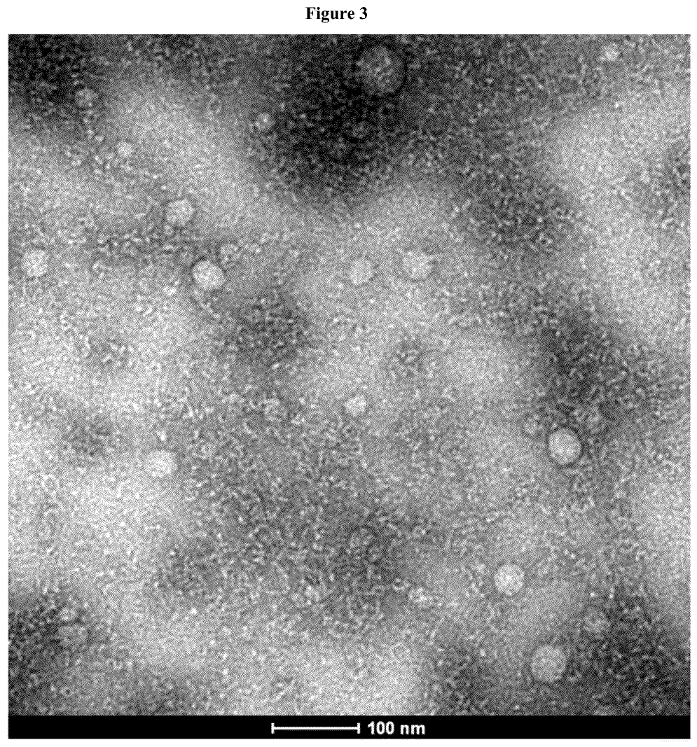

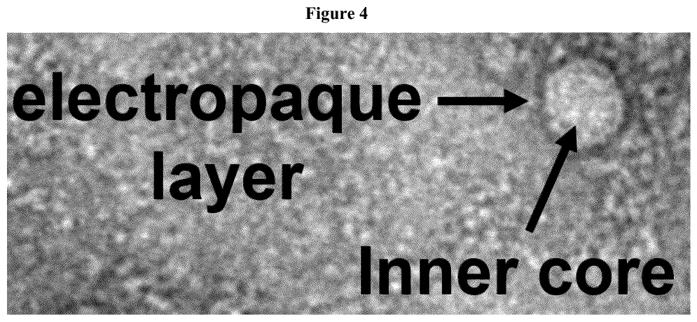

TEM images of Myr5A nanoparticles are shown FIG. 2, FIG. 3, and FIG. 4. The undulating forms and coarse grain appearance of these images are from the interaction between the trehalose and the uracyl acetate and represent an artifact from preparation that is purely aesthetic and does not interfere with the nanoparticle structure or how the nanoparticles are imaged beyond the undulating forms and coarse grain appearance.

### Example 10. Myr5A Imidazole Ketone Erastin (IKE) Nanoparticles for Adrenocortical Carcinoma (ACC)

This Example describes Myr5A imidazole ketone erastin (IKE) nanoparticles for treating adrenocortical carcinoma.

Myr5A IKE nanoparticles are prepared similar to previous Examples. Briefly, H295R cells (an ACC cell line) are plated at 10,000 cells per well in white 384-well plates in technical duplicates and incubated overnight. The cells are then treated with vehicle (DMSO) and Myr5A imidazole ketone erastin (IKE) nanoparticles. Cell death and anti-tumor markers are measured.

### Example 11. Synthesis of IKE Myr5A Nanoparticles

In this example, IKE Myr5A nanoparticles were synthesized.

Method: Myr5A was dissolved in water. IKE was dissolved in the solvents shown in the table below. The solution of IKE was added dropwise to the solution of Myr5A. The resulting mixture was left on heated stir plate at 80° C. for 30 minutes. Subsequently, the sample was spun down at 5000 rpm for 10 minutes.

Results: Using the dropwise method described above, IKE Myr5A nanoparticles were synthesized.

Pre-lyophilization: The average particle size, polydispersity index (PDI), and D90 of the pre-lyophilized IKE Myr5A nanoparticles were measured. The results are shown in the table below.

TABLE 15

| Molar ratio | | | | |
|---|---|---|---|---|
| of | | Average particle | | D90 |
| IKE:Myr5A | IKE Solvent | size (nm) | PDI | (nm) |
| 1:3 | DCM | 217.7 | 0.238 | 138 |
| 1:5 | DCM | 177.4 | 0.106 | 76.7 |
| 1:10 | DCM | 84.4 | Multimodal | 53.3 |
| 1:4 | DCM | 80.4 | 0.200 | 44.7 |
| 1:5 | DCM | 121.9 | 0.223 | 57.2 |
| 1:7 | DCM | 111.6 | Multimodal | 60.2 |
| 1:3 | DCM | 173.5 | 0.236 | 108 |
| 1:5 | DCM | 180.7 | 0.184 | 79.6 |
| 1:5 | ethanol | 261.6 | 0.054 | 126 |
| 1:5 | ethanol/DCM | 177.1 | 0.181 | 81.2 |
| 1:5 | acetone/DCM | 88.5 | 0.115 | 58.7 |
| 1:7 | acetone/DCM | 225.8 | 0.157 | 224 |
| 1:5 | ACN/DCM | 288 | 0.131 | 132 |
| 1:7 | ACN/DCM | 213.4 | 0.157 | 94.3 |

Post-lyophilization: The average particle size, PDI, D90, and Encapsulation Efficiency (EE) of the post-lyophilized IKE Myr5A nanoparticles were measured. The results are shown in the table below.

TABLE 16

| Molar ratio | | | | | Encapsulation |
|---|---|---|---|---|---|
| of | IKE Solvent | Average particle | | D90 | Efficiency |
| IKE:Myr5A | System | size (nm) | PDI | (nm) | (EE) |
| 1:3 | DCM | 70.7 | Multimodal | 80.4 | 15% at conc. |
| | | | | | 0.079 mg/mL |
| 1:5 | DCM | 67.7 | Multimodal | 81.6 | 23% at conc. |
| | | | | | 0.067 mg/mL |
| 1:10 | DCM | 8.5 | Multimodal | 106.9 | <1% at conc. |
| | | | | | 0.001 mg/mL |
| 1:4 | DCM | 102.2 | 0.380 | 82 | 54.6% at conc. |
| | | | | | 0.059 mg/mL |
| 1:5 | DCM | 106.8 | 0.190 | 66 | 40.4% at conc. |
| | | | | | 0.034 mg/ml |
| 1:3 | DCM | 59.4 | 0.187 | 43.3 | ~100% at conc. |
| | | | | | 0.076 mg/mL |
| 1:5 | DCM | 67.8 | 0.260 | 63.4 | ~100% at conc. |
| | | | | | 0.053 mg/mL |
| 1:5 | acetone | 80.5 | Multimodal | 170 | 25% at conc. |
| | | | | | 0.141 mg/mL |
| 1:5 | ACN | 43.3 | Multimodal | 92.6 | 13.3% at conc. |
| | | | | | 0.075 mg/mL |
| 1:5 | acetone:DCM | 109.4 | Multimodal | 58.1 | ~100% at conc. |
| | | | | | 0.663 mg/mL |
| 1:7 | acetone:DCM | 174 | 0.110 | 99.7 | ~100% at conc. |
| | | | | | 0.489 mg/mL |
| 1:5 | ACN:DCM | 7.0 | 0.186 | 105 | ~100% at conc. |
| | | | | | 0.185 mg/mL |

### Example 12. Synthesis of ML210 Myr5A Nanoparticles

In this example, ML210 Myr5A nanoparticles were synthesized.

Method: Myr5A was dissolved in water. ML210 was dissolved in the solvents shown in the table below. The solution of IKE was added dropwise to the solution of Myr5A. The resulting mixture was left on heated stir plate at 80° C. for 30 minutes. Subsequently, the sample was spun down at 5000 rpm for 10 minutes.

Results: Using the dropwise method described above, ML210 Myr5A nanoparticles were synthesized.

Pre-lyophilization: The average particle size, polydispersity index (PDI), and D90 of the pre-lyophilized ML210 Myr5A nanoparticles were measured. The results are shown in the table below.

TABLE 17

| Molar ratio of | | Average particle | | D90 |
|---|---|---|---|---|
| ML210:Myr5A | ML210 Solvent | size (nm) | PDI | (nm) |
| 1:5 | DCM | 199.7 | 0.232 | 112 |
| 1:5 | MeOH/DCM | 327.2 | 0.148 | 151 |
| 1:5 | acetone/DCM | 254.7 | 0.183 | 113.6 |

Post-lyophilization: The average particle size, PDI, D90, and Encapsulation Efficiency (EE) of the post-lyophilized IKE Myr5A nanoparticles were measured. The results are shown in the table below.

TABLE 18

| Molar | | Average | | | |
|---|---|---|---|---|---|
| ratio of | IKE | particle | | | |
| ML210: | Solvent | size | | D90 | Encapsulation |
| Myr5A | System | (nm) | PDI | (nm) | Efficiency (EE) |
| 1:5 | DCM | 193 | 0.205 | 110 | ~100% at |
| | | | | | 0.046 mg/mL |
| | | | | | (theoretical: |
| | | | | | 21%) |
| 1:5 | MeOH/ | 124.7 | 0.237 | 76.8 | ~100% |
| | DCM | | | | 0.047 mg/mL |
| | | | | | (theoretical: |
| | | | | | 21%) |
| 1:5 | acetone/ | 126.7 | Multimodal | 172.2 | ~100% at |
| | DCM | | | | 0.057 mg/mL |
| | | | | | (theoretical: |
| | | | | | 26%) |

### Example 13. Synthesis of Fmoc-MMAE Myr5A Nanoparticles

In this example, Fmoc-MMAE Myr5A nanoparticles were synthesized.

Method: Myr5A was dissolved in water. Fmoc-MMAE was dissolved in DCM. The solution of Fmoc-MMAE was added dropwise to the solution of Myr5A. The resulting mixture was left on heated stir plate at 80° C. for 30 minutes. Subsequently, the sample was spun down at 5000 rpm for 10 minutes.

Results: Using the dropwise method described above, 1:3 Fmoc-MMAE Myr5A nanoparticles were synthesized.

Pre-lyophilization: The Encapsulation Efficiency (EE) of the Fmoc-MMAE Myr5A nanoparticles were measured. The results are shown in the table below.

TABLE 19

| Molar ratio of | Molar ratio of Fmoc- | |
|---|---|---|
| Fmoc- | MMAE: | Encapsulation |
| MMAE:Myr5A | Myr5A + DHPC | Efficiency (EE) |
| 1:3 | N/A | 66% at conc. 0.290 mg/mL |
| 1:3 | 1:5 | 25% at conc. 0.320 mg/mL |
| 1:5 | 1:5 | 37% at conc. 0.540 mg/mL |

### Example 14. Synthesis of Fmoc-MMAE Myr5A DHPC Nanoparticles

In this example, Fmoc-MMAE Myr5A DHPC nanoparticles were synthesized.

Method: Myr5A was dissolved in water and DHPC was dissolved in DCM along with Fmoc-MMAE. The solution of Fmoc-MMAE and DHPC was added dropwise to the aqueous solution of Myr5A. The resulting mixture was left on heated stir plate at 80° C. for 30 minutes.

Results: Using the dropwise method described above, Fmoc-MMAE Myr5A DHPC nanoparticles were synthesized.

Pre-lyophilization: The average particle size, polydispersity index (PDI), and D90 of the pre-lyophilized ML210 Myr5A nanoparticles were measured. The results are shown in the table below.

TABLE 20

| Molar ratio | | | | |
|---|---|---|---|---|
| of Fmoc- | Molar ratio | Average | | |
| MMAE:Myr5A + | of Myr5A | particle | | D90 |
| DHPC | to DHPC | size (nm) | PDI | (nm) |
| 1:5 | 1:5 | 147.5 | 0.067 | 83.5 |

Post-lyophilization: The average particle size, PDI, D90, and Encapsulation Efficiency (EE) of the post-lyophilized IKE Myr5A nanoparticles were measured. The results are shown in the table below.

TABLE 21

| Molar ratio | Molar | | | | |
|---|---|---|---|---|---|
| of Fmoc- | ratio of | Average | | | Encapsulation |
| MMAE: | Myr5A to | particle | | D90 | Efficiency |
| Myr5A | DHPC | size (nm) | PDI | (nm) | (EE) |
| 1:5 | 1:5 | 144.4 | 0.245 | 85.9 | 31% at conc. |
| | | | | | 0.320 mg/mL |

### Example 15. Synthesis of Myr5A DHPC and Myr5A DMPC Nanoparticles

In this example, Myr5A DHPC and Myr5A DMPC nanoparticles were synthesized.

Method: Myr5A was dissolved in water. The solution of Myr5A was added to DHPC or DMPC in water. The resulting mixtures were left on heated stir plate at 70-75° C. for 30 minutes.

Results: Using the dropwise method described above, Myr5A DHPC (1:1) and Myr5A DMPC (1:1) nanoparticles were synthesized.

Pre-lyophilization: The average particle size, polydispersity index (PDI), and D90 of the pre-lyophilized Myr5A DHPC and Myr5A DMPC nanoparticles were measured. The results are shown in the table below.

TABLE 22

| | Molar ratio of | Average particle | | D90 |
|---|---|---|---|---|
| Lipid | Myr5A:lipid | size (nm) | PDI | (nm) |
| DHPC | 1:1 | 181 | 0.138 | 82.6 |
| DMPC | 1:1 | 264.4 | 0.118 | 119.3 |

Post-lyophilization: The average particle size, PDI, D90, and Encapsulation Efficiency (EE) of the post-lyophilized Myr5A DHPC and Myr5A DMPC nanoparticles were measured. The results are shown in the table below.

TABLE 23

| | Molar ratio of | Average particle | | D90 |
|---|---|---|---|---|
| Lipid | Myr5A:lipid | size (nm) | PDI | (nm) |
| DHPC | 1:1 | 84.7 | 0.233 | 56.9 |
| DMPC | 1:1 | 100.3 | 0.175 | 57.1 |

### Example 16. Synthesis of AD198 Myr5A DHPC Nanoparticles

In this example, AD198 Myr5A DHPC nanoparticles were synthesized.

Method: Myr5A was dissolved in water and AD198 together with DHPC were dissolved in DCM at different payload molar ratios (table 24). The solution of AD198 and DHPC was added dropwise to the solution of Myr5A and DHPC. The resulting mixtures was left on heated stir plate at 40° C. for 30 minutes.

Results: Using the dropwise method described above, AD198 Myr5A DHPC nanoparticles were synthesized.

Post-lyophilization: The average particle size, PDI, D90, and Encapsulation Efficiency (EE) of the post-lyophilized AD198 Myr5A DHPC nanoparticles were measured. The results are shown in the table below.

TABLE 24

| Molar | Myr5A to | | | | |
|---|---|---|---|---|---|
| ratio of | DHPC | Average | | | Encapsulation |
| AD198: | Molar | particle | | D90 | Efficiency |
| Myr5A | Ratio | size (nm) | PDI | (nm) | (EE) |
| 1:5 | 1:5 | 316.7 | Multimodal | 360 | 65% at conc. |
| | | | | | 0.922 mg/mL |
| 1:5 | 1:10 | 95.7 | Multimodal | 80.2 | 100% at conc. |
| | | | | | 1.922 mg/mL |
| 1:3 | 1:5 | 112.3 | 0.290 | 78.5 | 67% at conc. |
| | | | | | 0.794 mg/ml |
| 1:3 | 1:10 | N/A | N/A | N/A | N/A |

### Example 17. Synthesis of AD198 Myr5A DHPC Nanoparticles

In this example, AD198 Myr5A DHPC nanoparticles were synthesized.

Method: Myr5A was dissolved in water and AD198 together with DHPC were dissolved in DCM at a molar ratio of 1 to 3 (AD198:DHPC). The solution of AD198 and DHPC was added dropwise to the solution of Myr5A and DHPC. The resulting mixture was left on heated stir plate at 75° C. for 30 minutes.

Results: Using the dropwise method described above, AD198 Myr5A DHPC nanoparticles were synthesized.

Pre-lyophilization: The average particle size, polydispersity index (PDI), and D90 of the pre-lyophilized AD198 Myr5A DHPC nanoparticles were measured. The results are shown in the table below.

TABLE 25

| Molar ratio | | | | |
|---|---|---|---|---|
| of | Myr5A | Average | | |
| AD198: | to DHPC | particle | | D90 |
| Myr5A | Molar Ratio | size (nm) | PDI | (nm) |
| 1:3 | 1:5 | 189.1 | 0.219 | 122 |

Post-lyophilization: The average particle size, PDI, D90, and Encapsulation Efficiency (EE) of the post-lyophilized AD198 Myr5A DHPC nanoparticles were measured.

The results are shown in the table below.

TABLE 26

| Molar | Myr5A | Average | | | |
|---|---|---|---|---|---|
| ratio of | to DHPC | particle | | | |
| AD198: | Molar | size | | D90 | Encapsulation |
| Myr5A | Ratio | (nm) | PDI | (nm) | Efficiency (EE) |
| 1:3 | 1:5 | 116.8 | 0.235 | 95.4 | 77% at conc. |
| | | | | | 1 mg/mL |

### Example 18. Synthesis of AD198 Myr5A DHPC Nanoparticles

In this example, AD198 Myr5A DHPC nanoparticles were synthesized.

Method: Myr5A was lyophilized. Then Myr5A and DHPC (1:5 molar ratio) were dissolved in solution of ammonium acetate (pH 4.3) and ACN. AD198 was dissolved in DCM. The solution of AD198 was added dropwise to the solution of Myr5A and DHPC. The resulting mixture was left on heated stir plate at 75° C. for 30 minutes.

Results: Using the dropwise method described above, AD198 Myr5A DHPC nanoparticles were synthesized.

Pre-lyophilization: The average particle size, polydispersity index (PDI), and D90 of the pre-lyophilized AD198 Myr5A DHPC nanoparticles were measured. The results are shown in the table below.

TABLE 27

| Molar ratio | | | Average | | |
|---|---|---|---|---|---|
| of AD198: | | Myr5A | particle | | |
| Myr5A + | | and DHPC | size | | D90 |
| DHPC | Myr5A | Solvent | (nm) | PDI | (nm) |
| 1:3 | No | 10 mmol | 266.4 | 0.025 | 123 |
| | lyophilization | ammonium acetate | | | |
| | prior to | (pH 4.3) and ACN | | | |
| | synthesis | (10%) | | | |
| 1:3 | No | 50 mmol | 309 | 0.098 | 132 |
| | lyophilization | ammonium acetate | | | |
| | prior to | (pH 4.3) and ACN | | | |
| | synthesis | (10%) | | | |

Post-lyophilization: The average particle size, PDI, D90, and Encapsulation Efficiency (EE) of the post-lyophilized AD198 Myr5A DHPC nanoparticles were measured. The results are shown in the table below.

TABLE 28

| Molar ratio | | | Average | | |
|---|---|---|---|---|---|
| of AD198: | | Myr5A | particle | | |
| Myr5A + | | and DHPC | size | | D90 |
| DHPC | Myr5A | Solvent | (nm) | PDI | (nm) |
| 68.1 | Lyophilized | 10 mmol | 116 | 0.232 | 53.2 |
| | with | ammonium acetate | | | |
| | Trehalose | (pH 4.3) and ACN | | | |
| | | (10%) | | | |
| 1:3 | Lyophilized | 50 mmol | 73.1 | 0.218 | |
| | with | ammonium acetate | | | |
| | Trehalose | (pH 4.3) and ACN | | | |
| | | (10%) | | | |

### Example 19. Synthesis of IKE Myr5A Pal-KTTKS (SEQ ID NO: 46) (Acetate) Nanoparticles

In this example, IKE Myr5A Pal-KTTKS (SEQ ID NO: 46) (acetate) nanoparticles were synthesized.

Method: Myr5A and Pal-KTTKS (SEQ ID NO: 46) (acetate) (1:5 molar ratio) were dissolved in water. IKE was dissolved in DCM. The solution of IKE was added dropwise to the solution of Myr5A and Pal-KTTKS (SEQ ID NO: 46) (acetate). The resulting mixture was left on heated stir plate at 70-75° C. for 30 minutes.

Results: Using the dropwise method described above, IKE Myr5A Pal-KTTKS (acetate) nanoparticles were synthesized. The molar ratio of IKE to Myr5A+Pal-KTTKS (acetate) was 1:3.

Pre-lyophilization: The average particle size, polydispersity index (PDI), and D90 of the pre-lyophilized IKE Myr5A Pal-KTTKS (SEQ ID NO: 46) (acetate) nanoparticles were measured. The results are shown in the table below.

TABLE 29

| | Myr5A and Pal- | | | |
|---|---|---|---|---|
| Molar ratio | KTTKS (SEQ ID | Average | | |
| of | NO: 46) (acetate) | particle | | D90 |
| IKE:Myr5A | Solvent | size (nm) | PDI | (nm) |
| 1:3 | Water | 166.7 nm | 0.229 | 93.5 |

Post-lyophilization: The average particle size, PDI, D90, and Encapsulation Efficiency (EE) of the post-lyophilized IKE Myr5A Pal-KTTKS (SEQ ID NO: 46) (acetate) nanoparticles were measured. The results are shown in the table below.

TABLE 30

| | Myr5A and Pal- | Average | | | |
|---|---|---|---|---|---|
| Molar | KTTKS (SEQ ID | particle | | | |
| ratio of | NO: 46) (acetate) | size | | D90 | Encapsulation |
| IKE:Myr5A | Solvent | (nm) | PDI | (nm) | Efficiency (EE) |
| 1:3 | Water | 117.7 | 0.234 | 77.7 | 6.38 % at conc. |
| | | | | | 0.313 mg/mL |

### Example 20. Synthesis IR780 DHPC Myr5A Nanoparticles

In this example, IR780 DHPC Myr5A nanoparticles were synthesized.

Method: Myr5A was dissolved in water and DHPC was dissolved in DCM along with IR780. The solution of IR780 and DHPC was added dropwise to the aqueous solution of Myr5A. The resulting mixture was left on heated stir plate at 80° C. for 30 minutes.

Results: Using the dropwise method described above, IR780 Myr5A DHPC nanoparticles were synthesized.

Pre-lyophilization: The average particle size, polydispersity index (PDI), and D90 of the pre-lyophilized ML210 Myr5A nanoparticles were measured. The results are shown in the table below.

TABLE 31

| Molar ratio of | Molar ratio | Average | | |
|---|---|---|---|---|
| Myr5A + DHPC | of Myr5A | particle | | D90 |
| to IR780 | to DHPC | size (nm) | PDI | (nm) |
| 1:5 | 1:5 | 239.6 | 0.084 | 135 |

Post-lyophilization: The average particle size, PDI, D90, and Encapsulation Efficiency (EE) of the post-lyophilized IR780 Myr5A nanoparticles were measured. The results are shown in the table below.

TABLE 32

| | | Average | | | |
|---|---|---|---|---|---|
| Molar ratio of | Molar ratio | particle | | | |
| Fmoc- | of Myr5A | size | | D90 | Encapsulation |
| MMAE:Myr5A | to DHPC | (nm) | PDI | (nm) | Efficiency (EE) |
| 1:5 | 1:5 | 90.7 | 0.245 | 85.9 | 31% at conc. |
| | | | | | 0.320 mg/mL |

### EQUIVALENTS

Those skilled in the art will recognize, or be able to ascertain, using no more than routine experimentation, numerous equivalents to the specific embodiments described herein. Such equivalents are intended to be encompassed in the scope of the following claims.

1. A nanoparticle, comprising:
(a) a payload; and
(b) a conjugate having the structure of formula (I): 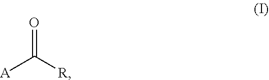
wherein
A is a peptide; and
R is C₁₋₄₀ alkyl, C₂₋₄₀ alkenyl, or C₂₋₄₀ alkynyl.

2. The nanoparticle of claim 1, wherein the nanoparticle has a particle size of about 5 nm to about 175 nm.

3. The nanoparticle of claim 1, wherein the nanoparticle has a payload:conjugate molar ratio of about 1:20 to about 1:1.

4. The nanoparticle of claim 1, wherein R is C₆₋₄₀ alkyl, C₆₋₄₀ alkenyl, or C₆₋₄₀ alkynyl.

5. The nanoparticle of claim 1, wherein R is:

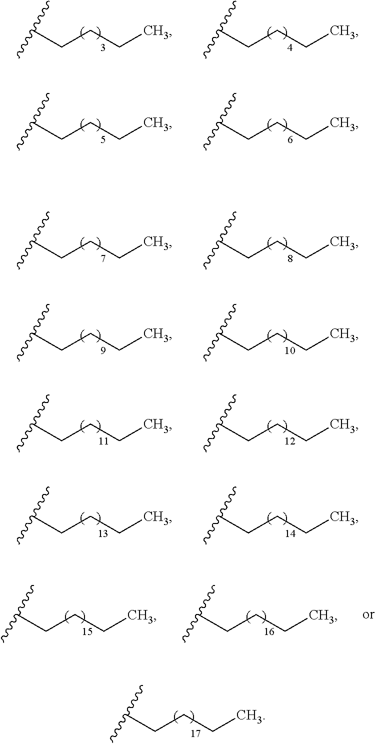

6. The nanoparticle of claim 1, wherein R is

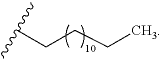

7. The nanoparticle of claim 1, wherein the peptide is an apolipoprotein.

8. (canceled)

9. The nanoparticle of claim 1, wherein the peptide is an apolipoprotein mimetic.

10. (canceled)

11. The nanoparticle of claim 9, wherein the apolipoprotein mimetic comprises an amino acid sequence having at least about 70% sequence identity to the amino acid sequence of any one of SEQ ID NOs: 1-45.

12. (canceled)

13. The nanoparticle of claim 1, wherein the payload is a therapeutic agent.

14. (canceled)

15. The nanoparticle of claim 13, wherein the therapeutic agent is an anthracycline, an anthracenedione, a bleomycin, or a mitomycin.

16. The nanoparticle of claim 13, wherein the therapeutic agent is an alkaloid, a monomethyl auristatin, a taxane, a quinazolinone, a cyclic dinucleotide, a macrocycle-bridged STING agonist, a xanthone, a adenosine derivative, a guanosine derivative, a cytidine derivative, a uridine derivative, or a thymidine derivative.

17.-19. (canceled)

20. The nanoparticle of claim 13, wherein the therapeutic agent is selected from the group consisting of cyclophosphamide, chlorambucil, cisplatin, etoposide, ametantrone, bleomycin, dactinomycin, daunorubicin, doxorubicin, epirubicin, idarubicin, mitomycin C, mitoxantrone, N-benzyladriamycin-14-valerate (AD198), valrubicin, docetaxel, monomethyl auristatin E (MMAE), fluorenylmethoxycarbonyl-monomethyl auristatin E (Fmoc-MMAE), monomethyl auristatin F (MMAF), paclitaxel, vinblastine, vincristine, vindesine, vinorelbine, bardoxolone methyl, curcumin, deferasirox, deferoxamine mesylate, erastin, imidazole ketone erastin (IKE), lapatinib,linagliptin, nordihydroguaiaretic acid (NDGA), pioglitazone, rosadustat, rosiglitazone, setanaxib, simvastatin, sorafenib, sulfasalazine, troglitazone, zileuton, RSL3, MHL162, MHL210, everolimus, rapamycin, ridaforolimus, sirolimus, temsirolimus, altretamine, bendamustine, busulfan, carboplatin, carmustine, dacarbazine, ifosfamide, lurbinectedin, lomustine, mechlorethamine, melphalan, oxaliplatin, temozolomide, thiotepa, trabectedin, camptothecin, 7-ethyl-10-hydroxycamptothecin (SN-38), exatecan, gimatecan, irinotecan, karenitecin, lurtotecan, rubitecan, silatecan, topotecan, diflomotecan, (S)-13-cyclobutyl-7-ethyl-7-hydroxy-9,12-dihydro-7H-cyclopenta[6,7]indolizino[1,2-b][1,3]dioxolo[4,5-g]quinoline-8,10-dione (S3925), cabazitaxel, eribulin, ixabepilone, tirbanibulin, niraparib, olaparib, pamiparib, rucaparib, talazoparib, veliparib, dimethylxanthone acetic acid (DMXAA or vadimezan), ADU-S100, E7766, MK-1454, acelarin, capecitabine, gemcitabine, sapacitabine, and ML210, or a pharmaceutically acceptable salt thereof.

21. The nanoparticle of claim 15, wherein the therapeutic agent is selected from the group consisting of N-benzyladriamycin-14-valerate (AD198), monomethyl auristatin E (MMAE), fluorenylmethoxycarbonyl-monomethyl auristatin E (Fmoc-MMAE), erastin, imidazole ketone erastin (IKE), rapamycin, rucaparib, dimethylxanthone acetic acid (DMXAA or vadimezan), acelarin, and ML210, or a pharmaceutically acceptable salt thereof.

22.-27. (canceled)

28. The nanoparticle of claim 1, further comprising a lipid.

29. The nanoparticle of claim 28, wherein the lipid is a phospholipid or a lipopeptide.

30. The nanoparticle of claim 29, wherein the phospholipid is phosphatidylcholine (PC), lysophosphatidylcholine (LPC), phosphatidic acid (PA), lysophosphatidic acid (LPA), phosphatidylethanolamine (PE), lysophosphatidylethanolamine (LPE), phosphatidylglycerol (PG), lysophosphatidylglycerol (LPG), phosphoinositides (PI), lysophosphatidylinositol (LPI), phosphatidylserine (PS), or lysophosphatidylserine (LPS).

31. (canceled)

32. (canceled)

33. The nanoparticle of claim 29, wherein the lipopeptide is a palmitoylated peptide.

34.-71. (canceled)

72. A pharmaceutical composition, comprising:
(a) a plurality of nanoparticles comprising:
(i) a payload; and
(ii) a conjugate comprising the structure of formula (I): 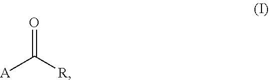
wherein
A is a peptide; and
R is C₁₋₄₀ alkyl, C₂₋₄₀ alkenyl, or C₂₋₄₀ alkynyl; and
(b) a pharmaceutically acceptable excipient.

73. The pharmaceutical composition of claim 72, wherein the pharmaceutical composition comprises less than about 5% impurities.

74.-109. (canceled)

110. A method of treating a disease or disorder in a subject in need thereof, comprising administering to the subject a therapeutically effective amount the pharmaceutical composition of claim 72.

111. (canceled)
